(19) United States
(12) Patent Application Publication
(10) Pub. No.: US 2025/0059127 A1
(43) Pub. Date: Feb. 20, 2025

(54) ADIABATIC RAPID PASSAGE AND SPIN COHERENCE TRANSFER MEDIATES EFFICIENT HYPERPOLARIZATION OF HETERONULCLEI IN PRODUCTS OF HYDROGENATION WITH PARAHYDROGEN

(71) Applicant: University of Florida Research Foundation, Inc., Gainesville, FL (US)

(72) Inventor: Clifford R. Bowers, Gainesville, FL (US)

(21) Appl. No.: 18/921,653

(22) Filed: Oct. 21, 2024

Related U.S. Application Data
(63) Continuation-in-part of application No. PCT/US2023/065873, filed on Apr. 18, 2023.
(60) Provisional application No. 63/363,287, filed on Apr. 20, 2022.

(51) Int. Cl. C07C 67/303; A61K 49/10

(52) U.S. Cl. CPC C07C 67/303; A61K 49/10; C07C 2523/46

(57) ABSTRACT

In one aspect, the disclosure relates to systems and methods for preparing precursor molecules including a hyperpolarized heteronucleus. The precursor molecules can be metabolites or derivatives. Heteronucleus hyperpolarization can be achieved by hydrogenating a carbon-carbon bond with parahydrogen, followed by transferring hyperpolarization from a parahydrogen to another hydrogen using adiabatic passage, after which an INEPT, selective INEPT, or MINERVA pulse sequence can be used to transfer hyperpolarization to a heteronucleus. Also disclosed are MRI contrast agents including the small molecules as well as a method of detecting a disease state associated with abnormal activity of a precursor molecule metabolite in a subject, the method including at least the step of administering a metabolite having a hyperpolarized heteronucleus to the subject and detecting the metabolite in the subject. In some aspects, the disclosed systems can incorporate an ultrasonic spray nozzle for forming droplets in order to better control the rate of substrate hydrogenation.

### CROSS-REFERENCE TO RELATED APPLICATIONS

This application is a continuation-in-part of International Application No. PCT/US2023/065873, filed Apr. 18, 2023, which claims the benefit of and priority to U.S. Provisional Application Ser. No. 63/362,287, having the title “ADIABATIC PASSAGE AND SPIN COHERENCE TRANSFER MEDIATES EFFICIENT HYPERPOLARIZATION OF HETERONULCLEI IN PRODUCTS OF HYDROGENATION WITH PARAHYDROGEN” filed on Apr. 20, 2022, the disclosure of which is incorporated herein in by reference in its entirety.

### STATEMENT REGARDING FEDERALLY SPONSORED RESEARCH OR DEVELOPMENT

This invention was made with government support under Grant Numbers 2108306, 1644779, and 1933723, awarded by the National Science Foundation. The government has certain rights in the invention.

### BACKGROUND

Parahydrogen based hyperpolarization is an efficient and inexpensive method for sensitivity-enhanced nuclear magnetic resonance (NMR) spectroscopy and magnetic resonance imaging (MRI). Thus, there is a need to develop technology related to this.

### SUMMARY

The present disclosure provides for methods for preparing a hydrogenation adduct molecule, precursor molecules comprising at least one hyperpolarized heteronucleus as described herein, contrast agents comprising the precursor molecule as described herein, systems and devices for producing a fluid sample comprising a hyperpolarized heteronucleus, and the like

The present disclosure provides for a method for preparing a hydrogenation adduct molecule comprising at least one hyperpolarized heteronucleus, the method comprising: (a) providing a supply of parahydrogen; (b) hydrogenating a precursor molecule comprising at least one carbon-carbon double bond or triple bond and at least one heteronucleus using the supply of parahydrogen to form a hydrogenated analyte comprising at least a first parahydrogen atom and a second parahydrogen atom, wherein, during hydrogenation, at least one carbon-carbon double bond is reduced to a carbon-carbon single bond, or wherein at least one carbon-carbon triple bond is reduced to a carbon-carbon double bond; (c) transferring spin from the first parahydrogen atom or the second parahydrogen atom to a third hydrogen atom and transferring spin from the third hydrogen atom to at least one heteronucleus to produce the hyperpolarized heteronucleus; or (d) transferring spin from the first or second hydrogen atom directly to the heteronucleus, in molecules where the third hydrogen atom is not present.

The present disclosure provides for a system for producing a fluid sample comprising a hyperpolarized heteronucleus, comprising: a precursor solution introduction device, wherein the precursor solution comprises a target substrate molecule and a catalyst; a reaction chamber, wherein the precursor solution introduction device is configured to be in fluid communication with the reaction chamber; a gas introduction system in communication with the reaction chamber, wherein the gas introduction system is configured to introduce parahydrogen into the reaction chamber, wherein the system is configured to contact the precursor solution with the parahydrogen, resulting in the formation of a hyperpolarized fluid sample; an adiabatic transport tube, wherein the adiabatic transport tube is configured to receive the hyperpolarized fluid sample from the reaction chamber and wherein hyperpolarized fluid sample is subjected to an increase in magnetic field, resulting in a transfer of hyperpolarization from at least one parahydrogen atom in the target substrate to a second hydrogen atom in the target substrate; and a sample chamber in fluid communication with the adiabatic transport tube, wherein the hyperpolarized fluid sample is subjected to a high magnetic field, resulting in transfer of hyperpolarization from the second hydrogen to a heteronucleus and generating a target molecule comprising the hyperpolarized heteronucleus.

The present disclosure provides for a fluid sample comprising a hyperpolarized heteronucleus produced by the system of any of the aspects.

The present disclosure provides for a method for detecting a disease state associated with abnormal concentration or abnormal activity of a precursor molecule metabolite in a subject, the method comprising: (a) administering the contrast agent of any of the aspects or the fluid sample any of the aspects to the subject; and (b) detecting the contrast agent or the target molecule in the subject.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows two different reaction schemes for producing 13C hyperpolarized metabolites like pyruvic acid. The chemical bonding unsaturation required for pH₂ addition is incorporated by synthesizing a vinyl or propargyl ester (or similar) derivative of the carboxylic acid of interest. After hydrogenation with para-enriched hydrogen (pH₂), spin order transfer (SOT) to the carbonyl ¹³C renders, after hydrolytic cleavage, a hyperpolarized molecule that was not directly producible by pairwise hydrogenation.

FIG. 2 illustrates the results of numerical density matrix calculations of the proton spin polarizations (in percent) in various molecules formed by hydrogenation with 100% pH₂ at a low magnetic field (strong coupling of all protons) followed by quasi-adiabatic transport to high magnetic field (weak coupling of chemically inequivalent protons). The parahydrogen sourced protons are indicated in red as H¹ and H².

FIG. 3 illustrates timing diagrams for coherence transfer after adiabatic longitudinal transport of ethyl and allyl ester adducts (e.g., EPd₃ or APd₂). After hydrogenation with pH₂ at low magnetic field, the esters are adiabatically transported to high field where either the INEPT or MINERVA radiowave pulse sequence is applied. For INEPT transfer from proton Zeeman order on allyl CH₂ protons (in AP or APd), coherence transfer is optimized using τ₁=(2J_{CH})⁻¹ and τ₂=τ₁/2. For CDH groups (e.g., APd₂), τ₁=τ₂=(2J_{CH})⁻¹ is used. In selective INEPT (sINEPT), frequency selective shaped π pulses are applied to the allyl methylene proton(s), H³. The MINERVA sequence is used for coherence transfer from bilinear spin order and is optimized with τ₁=(2J_{CH})⁻¹ and τ₂=(2J_{HH})⁻¹.

FIG. 4 illustrates density matrix calculations of ¹³C polarizations obtained by application of the MINERVA or INEPT pulse sequences to EPd₃, APd₂, APd, and AP after PASADENA or ALTADENA preparation. In the MINERVA simulations, τ₂=τ₁J_{CH}/J_{HH}. For INEPT and sINEPT, τ₂=τ₁ for APd₂ and τ₂=τ₁/2 for APd and AP. The simulations were performed using Kuprov's Spinach library running in MATLAB using the Zeeman-Hilbert formalism, which neglects τ₁ and τ₂ spin relaxation.

FIG. 5 illustrates the block diagram for an ultrasonic spray-injection hyperpolarization flow NMR system. The setup uses regulated heating of the precursor solution to promote a higher rate of hydrogenation. Each experimental trial commenced with the infusion of (e.g., 1.0 mL of the 10 mM/40 mM) catalyst/substrate precursor solution through an ultrasonic nozzle and into the reaction chamber at an optimized flow rate of (e.g., 5 mL/min). The optimal timings and flow rates will generally depend on the spin relaxation time of the hydrogenation adduct as well as the rate of the chemical hydrogenation reaction and the adiabaticity criterion for the magnetic field profile used and the Hamiltonian parameters of the molecule of interest. Before entering the nozzle, the precursor solution passes through a temperature-controlled heat exchanger. The heat exchanger, nozzle, and reaction chamber were maintained at either room temperature or elevated temperature (e.g., 70° C.). After a sufficient delay (e.g., 3 s) to allow a sufficient volume of liquid products to collect at the bottom of the funnel-shaped reactor outlet, the withdrawing syringe is activated to pull the product liquid into the NMR probe at the optimal flow rate of (e.g., 3 mL/min). BPR=back-pressure regulator (e.g., 5.17 bars).

FIGS. 6A-6D presents single transient hyperpolarization-enhanced ¹³C{¹H} NMR LACADENA-sINEPT spectra of APd acquired at 9.4 T after hydrogenations at (FIG. 6A) 20° C. and (FIG. 6B) 70° C., respectively, with 97% pH₂. The thermally polarized ¹³C NMR spectra (lower spectral traces) are enlarged for clarity. (FIG. 6C) Seven trials were acquired using the same precursor solution. The LACADENA-sINEPT signals increased monotonically over the course of the series, possibly due to increased catalyst activity with catalyst activation or temperature variations in the reaction chamber. The corresponding thermally polarized ¹³C spectra were acquired after waiting 10 min to allow for complete spin-lattice relaxation of hyperpolarization. The 2048 signal transients were accumulated using a 30° pulse and a recycle delay of 5 s (Ernst condition). (FIG. 6D) Timing diagram indicating the two steps of the SOT.

FIGS. 7A-7B show rotating frame eigenvalue correlation diagrams for model Hamiltonians of type C1 and C3 as a function of the static magnetic field with J̅₁₂=25 Hz and J̅₂₃=5 Hz (upper diagrams) or J̅₂₃=0 Hz (lower diagrams). (a) C1, {⊕₁, δ₂, δ₃}={0, +10, −10}ppm. (b) C3, {δ₁, δ₂, δ₃}={+10, −10, 0}ppm. The LAC is seen in the dashed red box. Orange, blue, and burgundy arrows indicate polarized single spin transitions of H¹, H² and H³, respectively. In the absence of a LAC, transitions of H³ remain unpolarized. The correlation diagram for C2 is analogous to C1. Curves were calculated using the SpinDynamica package²⁶ in Wolfram's Mathematica. FIG. 7C Density operators resulting from adiabatic passage from strong to weak coupling of idealized AMX spin systems, assuming J₁₂>>J₂₃ and J₁₃=0.FIG. 7D Zeeman order produced by adiabatic passage from strong to weak coupling for idealized constructs.

FIG. 8A shows simulated and experimental 300 MHz ¹H ALTADENA NMR spectra of allyl acetate in methanol-d4. A solution of propargyl acetate and Rh(dppb) catalyst was injected through an ultrasonic nozzle into a chamber pressured to 7 bar 50% pH₂. The liquid products were funneled into 1/16″ PEEK tubing and transported under a pressure difference collected in a 5 mm NMR sample tube installed inside the NMR probe at 7 Tesla. FIG. 8B shows simulated and experimental spectra originating from Boltzmann thermal equilibrium at 7 T, 300 K. FIG. 8C shows spectral simulation of the allyl group with and without deuteration at H⁵ and H⁶. A high degree of conversion to H³ Zeeman order at the expense of bilinear order with H¹. FIG. 8D shows an ALTADENA spectrum simulation of BPd4, which has no LAC, showing a very low intensity at H³ and a classic ALTADENA net alignment pattern.

FIGS. 8E-F present the 400 MHz ¹H ALTADENA experimental spectra (in red) and numerically simulated ¹H ALTADENA spectra (in blue) of AP and APd, respectively, obtained by hydrogenation of 8 mM propargyl pyruvate precursors with pH₂ using 2 mM Rh catalyst in the 2.4 mT fringe field followed by transport into the flow probe at a flow rate of 3 mL/min for detection at 9.4 T using a 90° RF pulse. The spectra have been normalized to the H² peak. FIG. 8G presents the 400 MHz ¹H ALTADENA spectra of AP (in blue) and APd (in red) obtained by hydrogenation as in (FIGS. 8E, 8F) but with 40 mM propargyl pyruvate precursors and 10 mM Rh catalyst. The H² peak in the spectrum of AP has been scaled to match the H² peak of the APd spectrum. FIG. 8H presents the thermally polarized spectra of the reaction product solutions of FIG. 8G, acquired by accumulation of 4 transients, and plotted on the same vertical axis as FIG. 8G.

FIG. 9 illustrates a continuous-flow heterogeneous hydrogenation apparatus used to implement the experimental spin order transfer protocols. The precursor solution is injected through an AF2400 tube-in-tube gas permeable membrane for bubble-free dissolution of parahydrogen into the precursor solution. The hydrogenation reaction occurs in the heterogeneous hydrogenation packed bed reactor. Once the liquid containing the hyperpolarized molecules has reached the NMR flow cell, the coherence transfer pulse sequence (INEPT, sINEPT, or MINERVA) is applied for spin order transfer to the spins of a heteronucleus of interest.

FIG. 10 illustrates an apparatus for closed-loop, continuous-flow heterogeneous hydrogenation of a precursor solution followed by adiabatic transport into the NMR probe for spin order transfer to a heteronucleus.

FIGS. 11A and 11B illustrates experimental results for the LACADENA-sINEPT experiment performed with Propargyl Pyruvate and d-Propargyl Pyruvate, respectively. The experimental conditions are provided in the figure. Carbon-13 NMR signal enhancements of 1629 and 3555 were observed, respectively, corresponding to absolute carbon-13 polarization levels of 2.11% and 4.60%, respectively. These results confirm the significant improvement in the carbon-13 polarization that results from deuteration of the propargyl CH group. Furthermore, the theoretical simulations confirm that an additional factor of two will be gained by single deuteration of the CH₂ group of the propargyl side-arm.

FIG. 12A illustrates Scheme 1, while FIG. 12B illustrates a timing diagram for LACADENA-sINEPT of allyl ester adducts (e.g., AP, APd, or APd₂). After hydrogenation with pH₂ at low magnetic field, the esters are adiabatically transported to high field where the sINEPT pulse sequence is applied. The 180° flip angle pulses on each channel are shaded; 90° flip angle pulses are unshaded. The final dashed 90°-y pulse on the ¹³C channel is removed for direct observation of the ¹³C transverse magnetization. Coherence transfer is optimized using τ₁=(2J_{CH})⁻¹ and τ₂=τ₁/2. For CDH groups (e.g., APd₂), τ₁=τ₂=(2J_{CH})⁻¹ is used. In sINEPT, frequency selective shaped π pulses (shaded in blue) are applied to the allyl methylene (H³) protons(s).

FIG. 13 illustrates the theoretical ¹³C polarizations obtained for LACADENA-INEPT or sINEPT pulse sequences applied after adiabatic transport of allyl ester parahydrogen adducts from the strong to weak proton coupling regimes, with and without selective deuteration of the allyl methylene group.

FIGS. 14A-14C illustrate the single transient hyperpolarization-enhanced 13C{1H} NMR LACADENA-sINEPT spectra of APd acquired at 9.4 T after hydrogenations at (FIG. 14A) 20° C. and (FIG. 14B) 70° C., respectively. The thermally polarized 13C NMR spectra were enlarged for clarity. (FIG. 14C) Seven trials were acquired for using the same precursor solution. The corresponding thermally polarized 13C spectra were acquired after waiting 10 min to allow for complete spin-lattice relaxation of hyperpolarization. The 2048 signal transients were accumulated using a 30° pulse and a recycle delay of 5 s (Ernst condition). The Ernst angle condition was satisfied for the allyl ester 13C T1=32 s.

FIG. 15 illustrates a block diagram of the ultrasonic spray injection reactor system interfaced to the flow NMR spectrometer. The tubing connecting the withdrawal syringe to the reaction chamber outlet, including the 60 μL flow cell, is initially prefilled with solvent, and the reaction chamber is pressurized to 6 bars with >90% pH₂. The precursor solution is infused through a copper heating coil into the ultrasonic nozzle (3.5 W, 120 kHz) into the chamber at a flow rate of 5 mL/min. Products accumulate at the bottom of the funnel-shaped chamber and are drawn into the NMR probe at precisely controlled syringe pump flow rates. BPR=back-pressure regulator (5.17 bars).

FIG. 16A illustrates slNEPT τ₁ dependence from thermal equilibrium to observable Sx where the maximum intensity is observed at 141 ms. FIG. 16B illustrates slNEPT τ₂ dependence from thermal equilibrium to Sx where the maximum intensity is observed at 71 ms. Total T values is the time in between the 90° pulses. The selective 180° pulses were set to 37 ms.

FIGS. 17A-17C illustrate hydrogenation of prop-2-yn-1-yl 2-oxopropanoate-1-¹³C (AP) at 20° C. FIG. 17A illustrates the reaction scheme. FIG. 17B illustrates 400 MHz single scan hyperpolarized ¹³C NMR spectrum acquired polarization transfer to allyl 2-oxopropanoate-1-¹³C (AP) via slNEPT. FIG. 17C illustrates thermally polarized ¹³C NMR spectrum acquired using a 30° pulse, relaxation delay of 5 seconds, and 2048 scans. The precursor solution consisted of 10 mM Rh(cod)(dppb)BF₄ and 40 mM propargyl ¹³C-pyruvate in acetone-d₆.

FIGS. 18A-18C illustrate the hydrogenation of prop-2-yn-1-yl 2-oxopropanoate-1-¹³C (AP) at 70° C. FIG. 18A illustrates the reaction scheme. FIG. 18B illustrates 400 MHz single scan hyperpolarized ¹³C NMR spectrum acquired after polarization transfer allyl 2-oxopropanoate-1-¹³C (AP) via slNEPT pulse sequence. FIG. 18C illustrates the thermally polarized ¹³C NMR spectrum acquired after relaxation of hyperpolarized signal with 30° pulse, relaxation delay of 5 seconds, and 2048 scans. The precursor solution consisted of 10 mM Rh(cod)(dppb)BF₄ and 40 mM prop-2-yn-1-yl 2-oxopropanoate-1-¹³C in acetone-d₆.

FIGS. 19A-19C illustrates the hydrogenation of prop-2-yn-1-yl-3-d 2-oxopropanoate-1-¹³C (APd) at 20° C. FIG. 19A illustrates the reaction scheme. FIG. 19B illustrates 400 MHz single scan hyperpolarized ¹³C NMR spectrum acquired after polarization transfer to (Z)-allyl-3-d 2-oxopropanoate-1-¹³C (APd) via slNEPT pulse sequence. FIG. 19C illustrates the thermally polarized ¹³C NMR spectrum acquired after relaxation of hyperpolarized signal with 30° pulse, relaxation delay of 5 seconds, and 2048 scans. The precursor solution consisted of 10 mM Rh(cod)(dppb)BF₄ and 40 mM APd in acetone-d₆.

FIG. 20 illustrates the stacked 400 MHz hyperpolarized ¹³C NMR spectrum acquired after polarization transfer to (Z)-allyl-3-d 2-oxopropanoate-1-¹³C (APd) via slNEPT pulse sequence. The precursor solution consisted of 10 mM Rh(cod)(dppb)BF₄ and 40 mM APd in acetone-d₆. Ultrasonic spray injection hydrogenation was performed at 20° C.

FIG. 21A illustrates the reaction scheme of APd. FIG. 21B illustrates ¹H thermally polarized NMR spectra acquired at 400 MHz (d1=120 s, 1 scan) (FIG. 21C) 400 MHz ¹H ALTADENA NMR spectra (d1=0.01 s, 1 scan). The precursor solution consisted of 10 mM Rh(cod)(dppb)BF₄ and 40 mM APd in acetone-d₆. Hydrogenation took place at room temperature and was achieved via ultrasonic spray injection.

FIG. 22 illustrates stacked 400 MHz thermally polarized ¹H NMR spectra acquired after ALTADENA type experiments of (Z)-allyl-3-d2-oxopropanoate-1-¹³C (APd). The highlighted region corresponds to the aromatic protons of the 1,4-bis(diphenylphosphino)butane (dppb) ligand. The precursor solution consisted of 10 mM Rh(cod)(dppb)BF₄ and 40 mM APd in acetone-d₆. Ultrasonic spray injection hydrogenation was performed at 70° C.

FIG. 23 illustrates inversion-recovery pulse sequence was used to measure the ¹³C spin-lattice relaxation time starting from thermal equilibrium nuclear spin polarization of allyl 2-oxopropanoate-1-¹³C. The τ₁ of the [1-¹³C] AP position is 32±0.3 s.

FIG. 24 illustrates provides precursor molecular structures that have been generalized by replacing a deuteron with a R′ group (e.g., D, CH₃, CH₂CH₃, methyl t-butyl ether).

FIG. 25 illustrates timing diagrams for coherence transfer after adiabatic longitudinal transport of ethyl and allyl ester adducts (e.g., EPd₃ or APd₂). After hydrogenation with pH₂ at low magnetic field, the esters are adiabatically transported to high field where either INEPT or MINERVA is applied. For INEPT transfer from proton Zeeman order on allyl CH₂ protons (in AP or APd), coherence transfer is optimized using τ₁=(2J_{CH})⁻¹ and τ₂=₁₁/2. For CDH groups (e.g., APd₂), τ₁=T2=(2J_{CH})⁻¹ is used. In selective INEPT (sINEPT), frequency selective shaped π pulses are applied to the allyl methylene proton(s), H³. The MINERVA sequence is used for coherence transfer from bilinear spin order and is optimized with τ₁=(2J_{CH})⁻¹ and τ₂=(2J_{HH})⁻¹.

Additional advantages of the present disclosure will be set forth in part in the description which follows, and in part will be obvious from the description, or can be learned by practice of the present disclosure. The advantages of the present disclosure will be realized and attained by means of the elements and combinations particularly pointed out in the appended claims. It is to be understood that both the foregoing general description and the following detailed description are exemplary and explanatory only and are not restrictive of the present disclosure, as claimed.

### DETAILED DESCRIPTION

In general, the present disclosure provides for methods for preparing a hydrogenation adduct molecule, precursor molecules comprising at least one hyperpolarized heteronucleus as described herein, contrast agents comprising the precursor molecule as described herein, systems and devices for producing a fluid sample comprising a hyperpolarized heteronucleus, and the like.

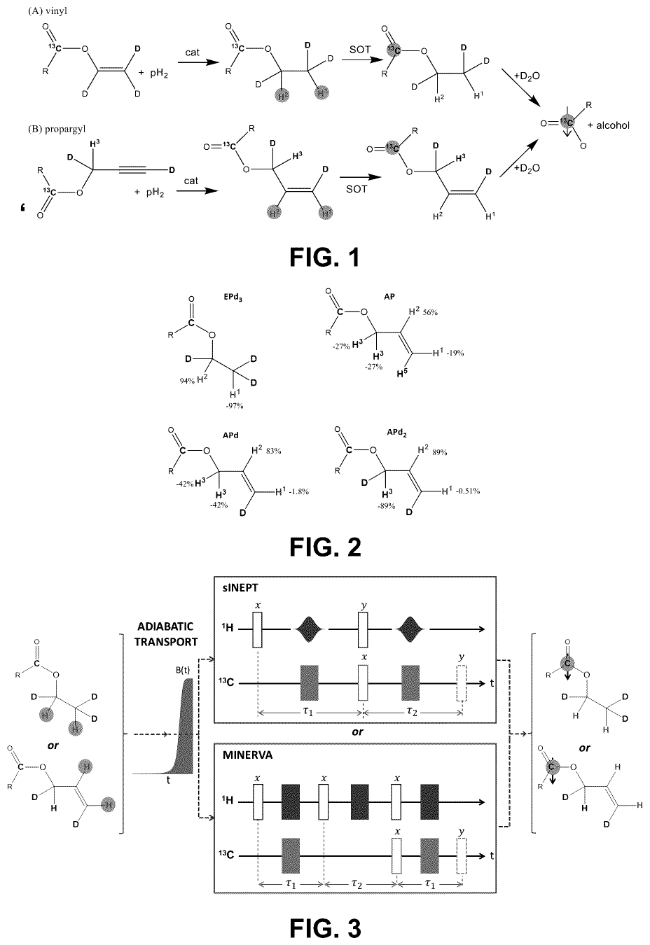

Nuclear spin hyperpolarization (HP) techniques have ushered in a new era of chemically selective magnetic resonance imaging (MRI) and localized nuclear magnetic resonance (NMR) spectroscopy. By inducing NMR signal enhancements that can exceed five orders of magnitude, metabolites and other biomolecules become visible despite their low in vivo concentrations, thereby enabling disease detection and treatment response monitoring without exposure to ionizing radiation. Parahydrogen (pH₂) is a convenient source of singlet nuclear spin order that can be rapidly transformed into MRI-observable proton hyperpolarization through symmetry-breaking hydrogenation chemistry. For metabolites like pyruvic acid, the requisite unsaturation for pH₂ addition can be incorporated by synthesis of the vinyl or propargyl ester, as exemplified in FIG. 1. After hydrogenation with pH₂, the non-equilibrium spin order is transferred to the carbonyl 13C to render, after hydrolytic cleavage, a hyperpolarized molecule that was not directly producible by pairwise hydrogenation. The NMR signals obtained from this hyperpolarized state can be many orders of magnitude stronger than the signals obtained from thermal equilibrium.

Disclosed herein is a versatile and efficient spin order transfer (SOT) process for producing HP ¹³C pyruvate and other carboxylic acids that is amenable to side arm hydrogenation (SAH) of vinyl and propargyl esters. Methods, devices, and instrumentation suitable for carrying out this process are also described.

For allyl ester adducts, the SOT process leverages level anti-crossing (LAC) mediated spin exchange with the initially unpolarized methylene proton(s) (See, Ferrer, M.-J.; Kuker, E. L.; Semenova, E.; Gangano, A. J.; Lapak, M. P.; Grenning, A. J.; Dong, V. M.; Bowers, C. R. Adiabatic Passage through Level Anticrossings in Systems of Chemically Inequivalent Protons Incorporating Parahydrogen: Theory, Experiment, and Prospective Applications. J Am Chem Soc 2022, 144 (45), 20847-20853, which is incorporated herein by reference).

For ethyl ester or similar adducts, the hydrogen atoms sourced from pH₂ are 2 and 3 bonds away from the target carbonyl ¹³C spin. The three bond heteronuclear J-coupling is strong enough to facilitate direct coherence transfer. For such adducts, the first step of the SOT process utilizes the standard ALTADENA effect. Hydrogenation is performed at a low magnetic, field (e.g., 5 mT) where all protons are strongly coupled, followed by adiabatic transport to high field where the weak coupling regime prevails for the chemically inequivalent protons. For a two proton adduct such as the d₃-ethyl ester (EPd₃ in FIG. 2), ALTADENA yields a density operator with the form

$${\hat{ρ}}_{f} = \hat{\frac{1}{4}} - {\hat{I}}_{z 1} {\hat{I}}_{z 2} \pm \frac{1}{2} ( {\hat{I}}_{z 1} - {\hat{I}}_{z 2} ) \qquad ( 1 )$$

Here, Î_{zi} denotes the z-component of the i^{th} proton spin angular momentum operator. Note that Equation (1) contains both linear and bilinear spin operator terms, which lends itself to several possible types of coherence transfer pulse sequences for transmitting the spin order to the heteronucleus (e.g., the carbonyl ¹³C spin), as described below.

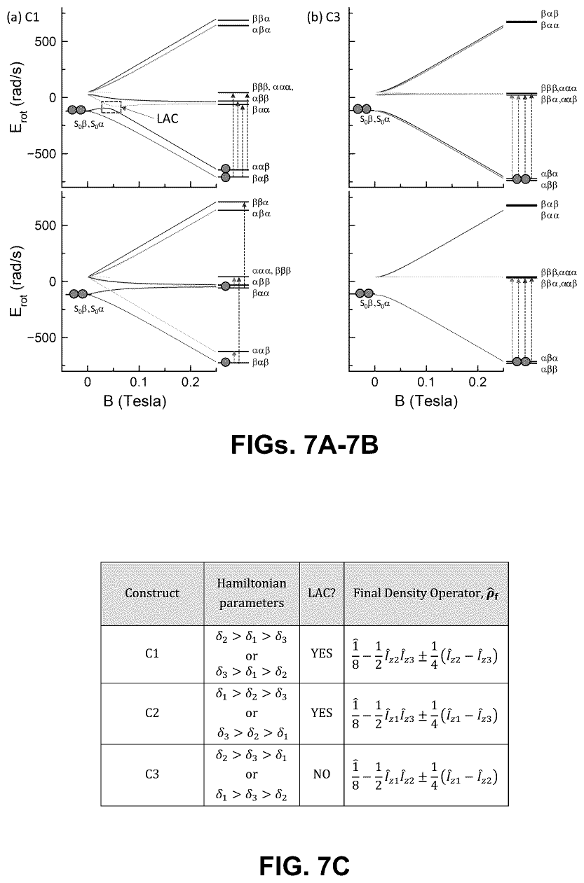

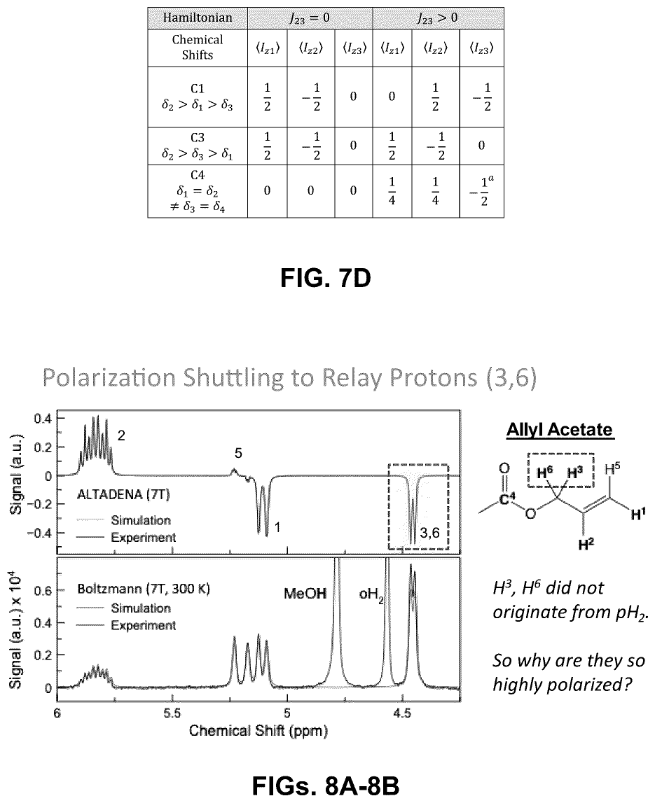

An analogous form is obtained in the three-proton allyl ester adducts incorporating pH₂. For example, for the APd₂ adduct in FIG. 2, where H¹ and H² are sourced from pH₂, adiabatic passage through a homonuclear LAC (referred to as LACADENA) produces approximately the same form of the density operator but with H³ replacing H¹: Density operators resulting from LACADENA for three idealized constructs with various combinations of the isotropic chemical shifts and J-couplings are presented in FIG. 7C, and the corresponding proton Zeeman orders for these same constructs are exhibited in FIG. 7D.

FIG. 2 summarizes the results of numerical calculations of the proton spin polarizations, Pᵢ=2Tr(Î_{zi}·ρ_{f}), obtained by adiabatic transport to the weak-coupling regime after hydrogenation (with pH₂) of vinyl and propargyl esters with various selective deuteration patterns. The high polarization of H³ in APd₂ derives from adiabatic passage through a LAC. The numerical calculations reveal that deuteration improves the efficiency of integration of pH₂ singlet order into the molecule, resulting in higher proton spin polarization, and by extension, higher ¹³C spin polarization after SOT by coherence transfer.

The occurrence of both forms of spin order in the ALTADENA and LACADENA experiments (i.e., linear and bilinear operator terms) affords a choice of coherence transfer pathways. The MINERVA NMR pulse sequence utilizes the bilinear terms (Î_{z1}Î_{z2} in EPd₃, Î_{z2}Î_{z3} in APd₂) while INEPT works on the Zeeman operator terms (Î_{z1}−Î_{z2} in EPd₃, or Î_{z2}−Î_{z3} in APd₂). This gives rise to a total of four different SOT scenarios, as illustrated in FIG. 3.

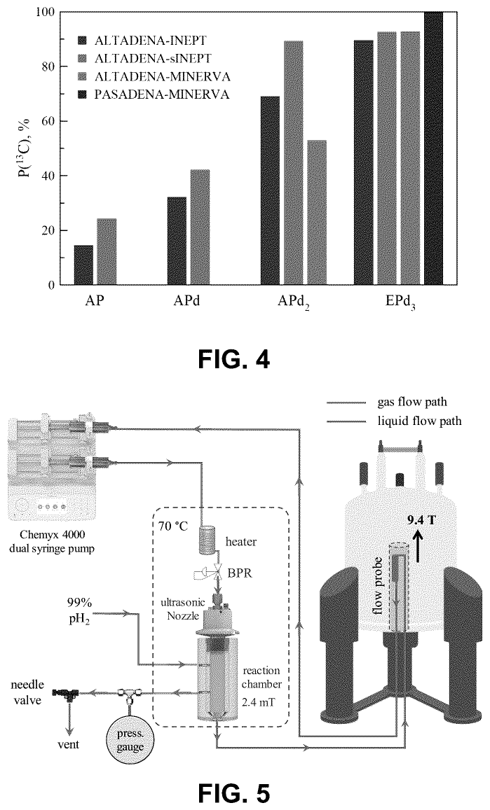

FIG. 4 summarizes the results of density matrix calculations of the ¹³C spin polarization P(¹³C) resulting from application of the MINERVA, INEPT and sINEPT coherence transfer pulse sequences for four different parahydrogen adducts after PASADENA, ALTADENA, or LACADENA preparation. In the PASADENA effect (parahydrogen and synthesis allow dramatically enhanced nuclear alignment), the hydrogenation is carried out at a high magnetic field where weak coupling among the protons spins is obtained. The PASADENA experiments generate only bilinear spin order Î_{z1}Î_{z2} for both types of esters (allyl and ethyl). Therefore, MINERVA is only applicable to ethyl ester type adducts, achieving P(¹³C)>95% for either ALTADENA or PASADENA preparation. Selective INEPT (sINEPT) after ALTADENA achieves a comparable performance, and standard (nonselective) INEPT yields P(¹³C)≈90%. The analogous simulations for APd₂ presented in FIG. 4 indicate generally lower overall theoretical efficiency, attributed to non-ideality of the LACADENA transformation. Nevertheless, P(¹³C)=−90% is seen in the LACADENA-sINEPT simulation. Lower theoretical polarizations of P(¹³C)=+50% and +70% were obtained with the MINERVA and non-selective INEPT sequences, respectively. Selective proton excitation is also beneficial for carboxylic acids with R groups bearing protons with appreciable coupling to the carbonyl ¹³C (e.g., acetate). In APd, where only H⁵ is deuterated, adiabatic passage through the LAC divides the terminal proton polarization between the pair of H³ protons in the allyl CH₂ group. This reduces the efficiency of INEPT by roughly a factor of two,^{10,17,18} as confirmed by the numerical simulations presented in FIG. 4. For APd, the INEPT simulation yields P(¹³C)=+32%, while sINEPT yields P(¹³C) −42%. The non-deuterated AP affords significantly lower polarization due to losses stemming from the substantial contribution of the H⁵ proton to the eigenstate in the strong coupling regime.

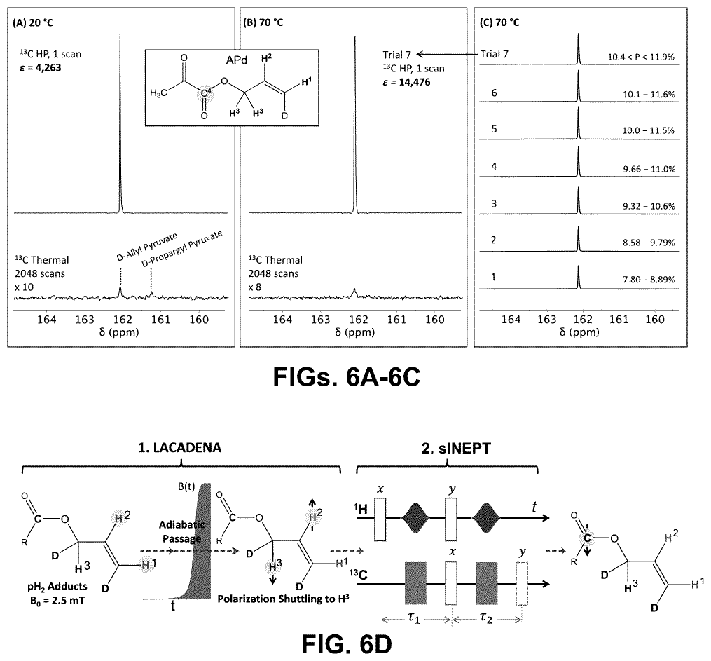

The LACADENA-sINEPT protocol was experimentally demonstrated with the pH₂ addition to [3-d]propargyl [1-¹³C]pyruvate using the apparatus diagramed in FIG. 5. A dual syringe pump (Chemyx 4000) was employed to synchronize infusion of the liquid precursor into the reaction chamber, and withdrawal of liquid hydrogenation adducts through the Varian 400 MHz flow NMR probe. The tubing connecting the withdrawing syringe to the reactor outlet, including the fluid path through the flow probe, was initially filled with acetone-d₆, and the reaction chamber was pressurized to 6 bars with 98% pH₂. The 20 mL infusing syringe was filled with 10 mM Rh(cod)(dppb)BF₄ and 40 mM propargyl ¹³C-pyruvate in acetone-d₆. The precursor solution was infused through a section of copper tubing wrapped around a heated brass block fitted with a thermocouple and a cartridge-type heating element to heat the precursor solution as it transited to the ultrasonic nozzle. The ultrasonic nozzle, vibrating at 120 kHz, produced a fine spray of precursor droplets with a mean diameter of about 13 μm. As the liquid containing the hydrogenation adducts was collected at the bottom of the reaction chamber, the withdrawing syringe was activated to draw the solution into the NMR detection coil. The withdrawing syringe flow rate was set to 3 mL/min to balance the losses due to spin relaxation against non-adiabaticity losses during the flow from the reactor to the NMR flow cell. Each of the hyperpolarized ¹³C spectra of [1-²H]allyl [1-¹³C]pyruvate (APd) presented in FIG. 6 were acquired after infusions of 1.0 mL of precursor solution through the ultrasonic nozzle. Hydrogenations were carried out at 70° C.

The disclosed process achieves highly efficient conversion of parahydrogen bilinear spin order into hyperpolarized magnetization of heteronuclei (i.e., any isotope other than proton). The conversion to Zeeman order of a third, initially unpolarized proton in the adduct molecule is induced via adiabatic passage through level anti-crossings (LACs) resulting from certain combinations of homonuclear proton-proton couplings and Zeeman interactions. In one aspect, this Zeeman order of the third spin, referred to as a the “relay” proton, can be easily converted to carbon-13 (or nitrogen-15, phosphorous-31, or fluorine-19) polarization by a simple coherence transfer pulse sequence with about 90 to 100% efficiency or 100% theoretical efficiency. In another aspect, the overall process is about 80% to 100% efficient. In one aspect, the disclosed method enhances a magnetic resonance signal for at least one heteronucleus by at least a factor of about 100,000 and up to 1,000,000.

In a still further aspect, this effect is identified to be active in a range of hydrogenation adducts, including certain unsaturated esters of ¹³C-pyruvate, a metabolite at the intersection of many biochemical reactions. In one aspect, in the disclosed process, following the adiabatic passage through the LAC, a simple coherence transfer pulse sequence is applied to achieve the theoretical maximum efficiency for SOT from pH₂ to ¹³C Zeeman hyperpolarization, providing a new hybrid route for preparing hyperpolarized pyruvate and other metabolites for sensitivity-enhanced magnetic resonance imaging of disease processes at the cellular level.

In one aspect, the disclosed process can be combined with known methods for complete hydrogenation with parahydrogen such as, for example the use of an ultrasonic spray-injection reactor, or the parahydrogen gas can be dissolved into a flowing stream of liquid precursor solution using an Telfon™ AF 2400 (amorphous fluoroplastic resin, or similar) gas permeable membrane. In another aspect, the disclosed process is a new method to convert the parahydrogen spin order to carbon-13 (or nitrogen-15, phosphorous-31, or fluorine-19) spin order in side-arm modified esters of metabolites such as acetate and pyruvate or any other carboxylic acid metabolite or dicarboxylic acid where conversion of the parahydrogen spin order is advantageous for increasing NMR or MRI signals.

In one aspect, by combining ultrasonic spray injection with the hybrid ALTADENA-sINEPT or LACADENA-sINEPT (selective insensitive nuclei enhancement by polarization transfer), continuous streams of ¹³C hyperpolarized metabolites can be produced and accumulated. In a further aspect, the entire process can be integrated into a single device for automated dispensing of ¹³C polarized metabolites and other contrast agents for sensitivity-enhanced biomedical magnetic resonance imaging.

Additional details are provided in the Examples.

In one aspect, disclosed herein is a method for preparing a hydrogenation adduct molecule including at least one hyperpolarized heteronucleus, the method including at least the steps of:
providing a supply of parahydrogen;
hydrogenating a precursor molecule including at least one carbon-carbon double bond or triple bond and at least one heteronucleus using the supply of parahydrogen to form a hydrogenated analyte comprising at least a first parahydrogen atom and a second parahydrogen atom, wherein, during hydrogenation, the at least one carbon-carbon double bond is reduced to a carbon-carbon single bond, or wherein the at least one carbon-carbon double bond is reduced to a carbon-carbon double bond;
transferring spin from the first parahydrogen atom or the second parahydrogen atom to a third hydrogen atom and transferring spin from the third hydrogen atom to the at least one heteronucleus to produce the hyperpolarized heteronucleus; or transferring spin from the first or second hydrogen atom directly to the heteronucleus, in molecules where the third hydrogen atom is not present.

In another aspect, the hydrogenating is conducted in a magnetic field of from about 0 to 1 μT, or 1 μT to 100 μT, or 100 μT to 2 T, 0.25 to 2 T, or of about 0, 0.25, 0.5, 0.75, 1, 1.25, 1.5, 1.75, or about 2 T, or a combination of any of the foregoing values, or a range encompassing any of the foregoing values.

In another aspect, the hydrogenation reaction can include contacting the precursor molecule and the supply of parahydrogen with a hydrogenation catalyst. In one aspect, the hydrogenation catalyst can be selected from or include Pt, Pd, Cu, Au, Ag, Rh, Ru, Ir, Ni, Sn, Co, Zn, Ce, Ti, Al, Fe, Si, or any combination thereof. In one aspect, the hydrogenation catalyst can be a Group VIII, IB, or IB transition metal-based catalyst including at least two different metals or can be Rh or an Rh alloy. In some aspects the hydrogenation catalyst can be in an insoluble nanoparticle form or in liquid form, as a suspension, dispersion, or emulsion.

In one aspect, the precursor molecule is an unsaturated ester of a Cₙ (n>0) carboxylic acid, where n can be 2 to 20, 2 to 10, 2 to 8, 2 to 6, 4 to 10, or 4 to 20. In another aspect, the unsaturated ester can be a vinyl ester or a propargyl ester. In one aspect, the precursor molecule can be vinyl acetate, propargyl pyruvate, or any combination thereof. In one aspect, one or more protons of the precursor molecule are replaced with deuteron(s) or R-groups to reduce or eliminate homonuclear spin couplings with the parahydrogen sourced protons.

In an aspect, at least one heteronucleus of the precursor molecule can be a carbon-13 atom, nitrogen-15, phosphorous-31, or fluorine-19. In a further aspect, at least one heteronucleus of the precursor molecule can be a carbon atom double bonded to an oxygen atom. In some aspects, the precursor molecule can further include at least one deuterium.

In one aspect, in the disclosed method, the ester can be hydrolyzed after hydrogenating the precursor molecule.

In an aspect, spin is transferred from the first parahydrogen atom or the second parahydrogen atom to the third hydrogen atom using adiabatic passage through a homonuclear level anticrossings. Further in this aspect, adiabatic passage can be accomplished via exposing the precursor molecule, after hydrogenation to form the precursor adduct molecule, to a continuously increasing magnetic field until the detection magnetic field, with a strength greater than about 0.25 T is reached.

In one aspect, spin can be transferred from the third hydrogen atom to at least one heteronucleus using a suitable coherence transfer pulse sequence, e.g., selective or non-selective insensitive nuclei enhancement by polarization transfer (INEPT) or the sequence known as MINERVA (see FIG. 3).

In one aspect, the process is at least 10% efficient, about 10% to 80%, about 80% to 100%, 80% to 99%, 80% to 90%, about 90 to 100%. Also disclosed herein are precursor molecules including at least one hyperpolarized heteronucleus produced by the disclosed method as well as contrast agents including the precursor molecules.

In an aspect, FIG. 24 provides precursor molecular structures that have been generalized by replacing a deuteron with a R′ group (e.g., D, an alkyl group (e.g., CH₃, CH₂CH₃, CH₂CH₂CH₃) alkyl ether, di-alkyl ether (e.g., methyl t-butyl ether)). In an aspect, group of atoms attached to a molecule, typically include of carbon, hydrogen, and oxygen atoms. In a particular embodiment, R′ can be a methyl t-butyl ether (MTBE), which is advantageous in that it that renders the side-arm hydrogenation adduct non-polar after hydrolysis, and this facilitates its removal from the aqueous phase by means of (1) gravimetric phase separation or (2) liquid-liquid separation relying on differences in surface wetting of a porous membrane.

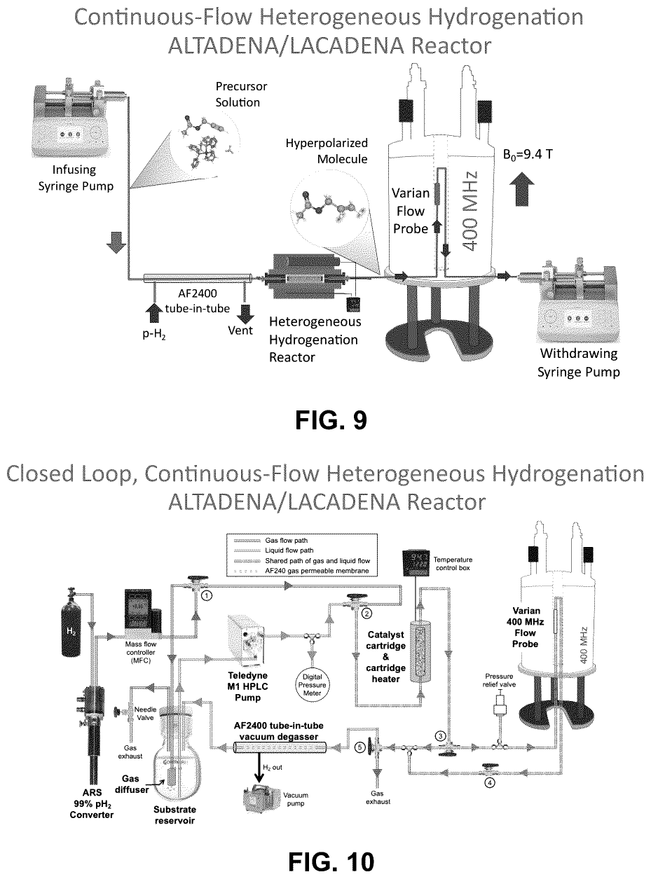

In one aspect, disclosed herein is a system (See, FIGS. 5, 9, and 10) for producing a fluid sample including a hyperpolarized heteronucleus, the system including:
a precursor solution introduction device, wherein the precursor solution comprises a target substrate molecule and a catalyst;
a reaction chamber, wherein the precursor solution introduction device is configured to be in fluid communication with the reaction chamber;
a gas introduction system in communication with the reaction chamber, wherein the gas introduction system is configured to introduce parahydrogen into the reaction chamber, wherein the system is configured to contact the precursor solution with the parahydrogen, resulting in the formation of a hyperpolarized fluid sample;
an adiabatic transport tube, wherein the adiabatic transport tube is configured to receive the hyperpolarized fluid sample from the reaction chamber and wherein hyperpolarized fluid sample is subjected to an increase in magnetic field, resulting in a transfer of hyperpolarization from at least one parahydrogen atom in the target substrate to a second hydrogen atom in the target substrate; and
a sample chamber in fluid communication with the adiabatic transport tube, wherein the hyperpolarized fluid sample is subjected to a high magnetic field, resulting in transfer of hyperpolarization from the second hydrogen to a heteronucleus and generating a target molecule comprising the hyperpolarized heteronucleus.

In a further aspect, the reaction chamber can include an ultrasonic nozzle, and wherein the precursor solution introduction device is configured to deliver the precursor solution to the surface of the ultrasonic nozzle, wherein the ultrasonic nozzle is configured to produce droplets of the precursor solution. In one aspect, the droplets of the precursor solution have an average diameter of from about 1 to about 50 μm, or of about 1, 5, 10, 15, 20, 25, 30, 35, 40, 45, or about 50 μm, or a combination of any of the foregoing values, or a range encompassing any of the foregoing values. In another aspect, the droplets can have a droplet distribution range of about 25 μm or less.

In one aspect, the precursor solution introduction device can be a syringe pump configured to deliver an amount of the precursor solution to the surface of the ultrasonic nozzle. In another aspect, the system further includes a detection device, and the sample chamber containing the fluid sample comprising a hyperpolarized heteronucleus is configured to fit into the detection device. In still another aspect, the detection device can be a nuclear magnetic resonance spectrometer or a magnetic resonance imaging device.

In one aspect, parahydrogen gas can be introduced into a flowing stream of liquid precursor solution containing the dissolved substrate molecule and dissolved or suspended catalyst using an Telfon™ AF 2400 (amorphous fluoroplastic resin, or similar) gas permeable membrane.

In one aspect, the hydrogenation reaction is catalyzed by a solid, insoluble heterogeneous catalyst material by passing the precursor solution containing the dissolved substrate molecule and dissolved parahydrogen through a packed bed reactor containing the solid catalyst. In such continuous-flow heterogeneous hydrogenation reactions, the parahydrogen gas is introduced by bubbling the parahydrogen through a reservoir of the precursor solution or by passing the precursor solution through a Telfon™ AF 2400 (amorphous fluoroplastic resin, or similar) gas permeable membrane.

In one aspect, the fluid sample is a homogeneous fluid including the target substrate molecule and a catalyst. In another aspect, the fluid sample is a heterogeneous fluid including the target substrate molecule and a particle that includes a catalyst. In one aspect, the catalyst can be a Group VIII, IB, or IB transition metal-based catalyst including at least two different metals, or may not include a metal. In still another aspect, the catalyst can include at least one of Pt, Pd, Cu, Au, Ag, Rh, Ru, Ir, Ni, Sn, Co, Zn, Ce, Ti, Al, Fe, Si, O, or any combination thereof.

In one aspect, the catalyst can be a nanoparticle having a diameter of a single metal atom to about 0.5 nm, or 0.5 nm to 1 nm, or 1 nm to 10 nm, or 10 nm to about 500 nm, or of about 10, 50, 100, 150, 200, 250, 300, 350, 400, 450, or about 500 nm, or a combination of any of the foregoing values, or a range encompassing any of the foregoing values. In some aspects, the catalyst can be in liquid form. In one aspect, the catalyst can be Rh or an Rh alloy, and can be supported, tethered, a ligand stabilized in solution, or any combination thereof.

In another aspect, the target substrate molecule can be a metabolite or a derivative thereof. In one aspect, the metabolite or derivative thereof can be administered in vitro or to a subject in vivo. In some aspects, the metabolite or derivative thereof can be an unsaturated ester of a C₁ to C₄ carboxylic acid or C₁ to C₈ carboxylic acid or C₁ to C₁₂ carboxylic acid. In another aspect, the unsaturated ester can be a vinyl ester or a propargyl ester. In one aspect, the metabolite or derivative thereof can be vinyl acetate, propargyl pyruvate, or any combination thereof. In one aspect, the at least one heteronucleus of the precursor molecule is a carbon-13, nitrogen-15, phosphorous-31, or fluorine-19 atom.

In one aspect, in the disclosed system, the precursor solution introduction device is configured to introduce precursor solution over a range of speeds, wherein a specific introduction speed can be selected prior to introducing the precursor solution, and wherein the introduction speed of the precursor solution is correlated to an amount of hyperpolarization in the fluid sample.

In one aspect, disclosed herein is a method for detecting a disease state associated with abnormal concentration or abnormal activity of a precursor molecule metabolite in a subject, the method including at least the steps of:
(a) administering the contrast agent as described herein or the fluid sample as described herein to the subject; and
(b) detecting the contrast agent or the target molecule in the subject.

In some aspects, the method further includes the step of administering one or more additional contrast agents to the subject, wherein the one or more additional contrast agents can be administered sequentially or simultaneously with the contrast agent or target molecule. In some aspects, detecting is accomplished using magnetic resonance imaging.

### Ultrasonic Spray Nozzle

In some aspects, hyperpolarized parahydrogen can be generated using an ultrasonic spray nozzle as described below.

Embodiments of the present disclosure provide for systems and methods of making hyperpolarized fluid samples. An embodiment of the present disclosure includes methods of making hyperpolarized fluid sample (e.g., hyperpolarized target molecules), which can be analyzed using a detection device. In general, the hyperpolarized fluid sample can be made by exposing a precursor solution (e.g., including a fluid, target molecules, and a catalyst) to parahydrogen. The interaction and/or chemical reaction between the molecules in the precursor solution and the parahydrogen in the presence of an appropriate homogeneous or heterogeneous catalyst can produce a hyperpolarized fluid sample that includes hyperpolarized protons or heteronuclei on the target molecules, including those metabolites and biomarkers that have been modified by attachment of a temporary ester sidearm containing a double or triple carbon-carbon bond that is chemically removed following the spin order transfer from the hydrogenated sidearm proton spins to observable hyperpolarization of heteronuclei.

In general, the method of making the hyperpolarized fluid sample can include making droplets of a particular size having a particular (e.g., narrow) size distribution using ultrasonic energy. The droplets, which have a very high surface area compared to those produced by bubbling or non-ultrasonic spray-injection methods, are then exposed to gaseous parahydrogen. The precursor, catalyst, and parahydrogen interact and/or react to form the hyperpolarized fluid sample, where a portion (about 50 to 100%) of the target molecules are hyperpolarized. In an aspect, the target molecule acquires one or more magnetized protons or hydrogen nuclei from parahydrogen by chemical exchange. In another aspect, the spin order of parahydrogen is transferred to the spins in a target molecule without any chemical hydrogenation of the target molecule, as occurs in the hyperpolarization phenomena commonly known as SABRE (Signal Amplification by Reversible Exchange), SWAMP (Surface Waters Are Magnetized from Parahydrogen), and NEPTUN (Nuclear Exchange Polarization by Transposing Unattached Nuclei).

In an aspect, embodiments of the present disclosure include a system for making the hyperpolarized fluid sample. In general, the system is configured to expose the parahydrogen to a precursor solution in the form of droplets (e.g., formed into droplets or an aerosol using an ultrasonic nozzle) leading to the production of the hyperpolarized fluid sample (e.g., hyperpolarized target molecules in a neat liquid or solution) that is collected, chemically processed, and analyzed or imaged. The system can include a temperature and pressure-controlled reaction chamber interfaced with a precursor introduction device, a gas introduction device, a collection and storage device, and optionally a detection device. The precursor introduction device introduces it into the reaction chamber that includes the ultrasonic nozzle. The gas introduction device can be configured to introduce the parahydrogen to the reaction chamber, where the droplets and the parahydrogen interact to form the hyperpolarize fluid sample, which can be collected, transferred to a storage device, and then introduced to the detection device, or administered in-vivo for subsequent detection by magnetic resonance imaging or spectroscopy. These collection and storage devices may be situated in a magnetically shielded container or zone, and may include appropriate static or alternating fields to effectuate the conversion of proton spin order into hyperpolarization of heteronuclei. The hyperpolarized fluid sample can be made in batch mode or in a continuous mode.

Now having described the methods and systems generally, additional details regarding the methods and systems are provided.

In an embodiment, the system can include a precursor introduction device that includes a precursor solution (e.g., the fluid containing the target molecules and the catalyst). The precursor introduction device is configured to be in fluidic communication with a reaction chamber. The reaction chamber can be operated at a temperature of about 25° C. to 300° C. and a pressure between about 1 bar to 100 bar. The reaction chamber includes an ultrasonic nozzle that has a surface. The sample introduction device is configured to deliver the sample to the surface of the ultrasonic nozzle. An ultrasonic nozzle may include a titanium horn body, a crystal/ceramic element with piezoelectric properties protected within a stainless-steel rear and front housing. Piezoelectric elements can include crystalline materials such as quartz, gallium orthophosphate, langasite, lithium tantalate, lithium niobate. Alternatively, piezoelectric ceramics that have been reported are barium titanate, potassium niobate, sodium tungstate, and the most commonly used lead zirconate titanate. Active and ground electrodes on the faces of the piezoelectric elements allow for an electrical connection to an ultrasonic generator. A liquid feed tube is situated through the titanium horn body to the tip of the atomizing surface. The atomizing surface shape can be conical, focused, or flat depending on the desired spray pattern. The production of the droplets results from the inverse piezoelectric effect, where high frequency (acoustic) sound waves are converted to mechanical energy to produce a vibrating surface. Transduction of the vibrations into the liquid phase produces standing waves in the liquid precursor solution and the amplitude of the surface increases until the liquid film becomes unstable and collapses into a fine mist of uniformly sized droplets. The ultrasonic nozzle can operate at a frequency ranging from about 20 kHz to 3 MHz to produce droplets of the precursor solution having diameters between 1 and 50 microns, depending on the operating frequency of a given nozzle. The high surface-to-volume ratio of droplets formed by the ultrasonic nozzle favors rapid diffusion of gaseous parahydrogen across the liquid/gas interface and into the interior of the liquid droplet within a relatively short timescale. The droplet diameter and size distribution can be tailored to maximize the hydrogenation reaction rate, conversion, and resultant hyperpolarization level in the collected fluid sample. The reaction chamber can have a volume sufficient to produce the desired volume of hyperpolarized fluid sample for in-vivo use, for example. In this regard, the reaction chamber has a volume of about 1 to 15 mL. The reaction chamber can be made of materials such as stainless steel, aluminum, polysulfone, vespel, polytetrafluoroethylene (PTFE), perfluoroalkoxy alkane (PFA), or polyether ether ketone (PEEK). In an aspect, the ultrasonic nozzle is located at the top of the reaction chamber and a collection vessel is located at the bottom of the reaction chamber to collect the droplets as they move through the reaction chamber. The reaction chamber can have a length of about 2 to 6 inches and a width or diameter of about 1 to 3 inches.

The parahydrogen is introduced to the reaction chamber using a gas introduction system that is in gaseous communication therewith. The gas introduction system can be configured to controllably introduce parahydrogen into the reaction chamber. The gas introduction device can include appropriate equipment to acquire (if part of a different system) and/or flow the parahydrogen to the reaction chamber. For example, the gas introduction system can include tubing, flow valves, pressure gauges, pressure regulators, syringe pumps, thermocouples, flow meters, and the like to control introduction into the holding vessel through the inlet port.

As briefly stated above, the system also includes the detection device that is in fluidic communication with the collection vessel or structure of the reaction chamber. The system is configured to transfer the hyperpolarized fluid sample from the reaction chamber to the detection device. The time between the formation of the droplets to analysis of the hyperpolarized fluid sample is about 100 ms to 2 s or less than about 16 seconds. The system is designed to deliver the hyperpolarized liquid on a timescale that is short compared to the spin-lattice relaxation time of the protons or heteronuclei. When the hyperpolarized product is produced in a singlet state, pseudo-singlet state, or other symmetry protected spin state comprised of more than two spins, the delivery to the detection device is performed on a timescale that is short compared to the lifetime of that spin state. The detection device can be a nuclear magnetic resonance device.

The liquid precursor introduction device can be a device that can inject a measured amount of the liquid precursor into the reaction chamber. In an aspect, the liquid precursor introduction device can be a syringe pump or HPLC pump configured to deliver a first amount of the liquid precursor solution to the surface of the ultrasonic nozzle.

In an aspect, the sample can be a homogeneous fluid including the solvent, the substrate molecule, and a catalyst, where the catalyst is dissolved in the fluid. In another aspect, the sample, the sample is a heterogeneous fluid including the target molecule and a particle comprising a catalyst, where the catalyst is insoluble in the fluid or is in the form of a suspension or emulsion of nanoparticles.

In an aspect, the fluid can be an aprotic solvent. The aprotic solvent can include: dioxane, nitromethane, acetonitrile, acetone, dichloromethane, or a combination thereof. In an aspect, the aprotic solvent can be a perdeuterated and partially deuterated form of each of the solvents listed above or herein. In an embodiment, the fluid is perdeuterated or partially deuterated water and is diluted in the aprotic solvent. In an embodiment, the fluid is biphasic, including immiscible polar and non-polar liquids.

In an aspect, the targeted molecule can include a functional group such as: —OH containing molecules, molecules including an amide or an amino group, an amino acid, a sugar, a carboxylic acid, a combination thereof, or any other moiety with an exchangeable proton. The target molecules can be any molecule. The targeted molecule can be side-arm modified metabolites such as allyl or propargyl esters of acetate, pyruvate, or arginine, or hydrogenation substrates yielding fumarate or succinate upon hydrogenation and a combination thereof.

### Catalyst

The catalyst can be a solid (heterogeneous) or liquid (homogeneous). The catalyst can be a compound that is soluble in the fluid. The catalyst can be a metal-based compound or a non-metal based compound. In another aspect, the catalyst is insoluble or substantially insoluble in the fluid. In an embodiment, the catalyst can be a Group VIII, IB, or IB transition metal-based catalyst including one or more metals (e.g., a bimetallic catalyst). In an aspect, the catalyst can include a one or more of the following: Pt, Pd, Cu, Au, Ag, Rh, Ru, Ir, Ni, Sn, Co, Zn, Zr, Ce, Ti, Al, Fe, Si or O. In an embodiment, at least one of the following are included in the catalyst: Pt, Pd, Cu, Au, Ag, Rh, Ru, Ir, Co, Fe, Ni, or L, where L is any lanthanide. In an aspect the catalyst is a metal complex including one or more of the metals described herein. For example, the liquid catalyst can include Rh(cod)(dppb)BF₄, Rh(nbd)(dppb)BF₄, [RuCp*(CH₃CN)₃]PF₆, or other cationic hydrogenation catalyst analogs. In another aspect, the catalyst is a solid and can be one or more of the following alloy or intermetallic compositions: PtSn, Pt₃Sn, PtPd, PtBi, PtZn, Pt₃Zn, PtRu, PtRh, PtPb, Pt₃Co, Pt₃Ti, Pt₃V, Pt₃Ni, PtAu, PtFe, PtCu, PtGe, Ptlr, PdCu, AuCu, CuFe, FeMnCu, CuNi, CuRu, CuCo, CuAg, AuPd, PdNi, PdFe, PdRu, PdSn, PdBi, PdPb, AgPd, PdCo, PdMn, Pdlr, RhCo, RhAg, RhFe, RhGe, RhNi, RhRe, RhSn, RuCo, RuSn, RuAg, NiRu, RuCr, IrNi, Colr, or AuAg. The catalyst can be a nanoparticle having a longest dimension (e.g., diameter) of about <1 nm to 20 nm. The metal or bimetallic nanoparticles may be supported on an oxide (e.g. SiO₂ (e.g. SBA-15, MCM-41), Al₂O₃, TiO₂) or it may include a metal organic framework (MOF) or covalent organic framework (COF) materials. The metal or bimetallic nanoparticles may include a ligand stabilized metal nanoparticle suspension, dispersion, or emulsion. In an embodiment, the catalyst does not contain any metal atoms. The catalyst may be an alloy, intermetallic compound, comprised of separated single atoms, single sites, or metal clusters that may or may not undergo metal-support interactions to stabilize the catalyst structure to prevent agglomeration and sintering.

The amount of catalyst in the precursor solution and in each droplet should be sufficient to provide enough active sites to accommodate hyperpolarization of the desired number of target molecules. Ideally, the total number of active sites of the catalyst in the volume of precursor solution that is introduced into the reaction chamber should be sufficient to allow complete conversion of all precursor molecules to hyperpolarized target molecules. Concentrations of precursor, parahydrogen, and catalyst are appropriate for hyperpolarization either by exchange of one or more magnetized protons from adsorbed parahydrogen or by non-hydrogenative mechanisms. In the case of catalyst nanoparticles, the amount of catalyst required can depend on the particle size, as the surface to volume ratio scales as 1/r, where r is the particle radius (assuming a spherical particle shape), the surface composition, and the rate of exchange, as well as the type of catalyst.

The system can include multiple different configurations includes one or more reaction chambers and/or one or more ultrasonic nozzles. For example, the reaction chamber can include multiple ultrasonic nozzles and/or the system can include multiple reaction chambers that are interfaced with the same collection or detection device. The precursor introduction system can direct the flow of the precursor solution to one or more reaction chambers and/or one or more ultrasonic nozzles. Depending upon the type and/or amount of target molecules, the configuration of the system can be adjusted to produce the desired results.

In another embodiment, the present disclosure provides for methods of collecting, storing, processing, purifying, redistributing nuclear spin order, and detecting the protons spins and heteronuclei of hyperpolarized molecules produced by ultrasonic spray injection into parahydrogen. The method can be implemented using the systems provided herein. The method can be combined with established methods for converting proton spin hyperpolarization derived from parahydrogen into hyperpolarization of heteronuclei. In an aspect, the method can include delivering a first amount of a precursor solution to a surface of the ultrasonic nozzle using a syringe pump and then producing droplets of the sample using an ultrasonic frequency from the ultrasonic nozzle. The method can include producing droplets of the precursor solution using an ultrasonic frequency (e.g., about 20 kHz to 3 MHz) to produce droplets that have an average diameter of about 1to 50 microns in a droplet distribution range of about 25 microns or less or as otherwise described herein. The droplets are mixed with the parahydrogen to initiate a chemical reaction or polarization transfer process that results in a hyperpolarized fluid sample. Subsequently, the hyperpolarized fluid sample is collected and can be analyzed using a detection device such as an NMR or magnetic resonance imaging system at high or low magnetic fields or zero magnetic field. The extension of this embodiment to in vivo administration and imaging of the hyperpolarized sample for detection or monitoring of metabolism, disease detection, and monitoring is also envisioned.

The following illustrates aspects and various combinations of aspects of the present disclosure.

Aspect 1. A method for preparing a hydrogenation adduct molecule comprising at least one hyperpolarized heteronucleus, the method comprising: (a) providing a supply of parahydrogen; (b) hydrogenating a precursor molecule comprising at least one carbon-carbon double bond or triple bond and at least one heteronucleus using the supply of parahydrogen to form a hydrogenated analyte comprising at least a first parahydrogen atom and a second parahydrogen atom, wherein, during hydrogenation, at least one carbon-carbon double bond is reduced to a carbon-carbon single bond, or wherein at least one carbon-carbon triple bond is reduced to a carbon-carbon double bond; (c) transferring spin from the first parahydrogen atom or the second parahydrogen atom to a third hydrogen atom and transferring spin from the third hydrogen atom to at least one heteronucleus to produce the hyperpolarized heteronucleus; or (d) transferring spin from the first or second hydrogen atom directly to the heteronucleus, in molecules where the third hydrogen atom is not present.

Aspect 2. The method of any of the aspects, wherein the hydrogenating is conducted in a magnetic field of from about 0 to about 2 T.

Aspect 3. The method of any of the aspects, wherein the hydrogenating comprises contacting the precursor molecule and the supply of parahydrogen with a hydrogenation catalyst.

Aspect 4. The method any of the aspects, wherein the hydrogenation catalyst comprises Pt, Pd, Cu, Au, Ag, Rh, Ru, Ir, Ni, Sn, Co, Zn, Ce, Ti, Al, Fe, Si, or any combination thereof.

Aspect 5. The method of any of the aspects, wherein the hydrogenation catalyst is a Group VIII, IB, or IB transition metal-based catalyst including at least two different metals.

Aspect 6. The method of any of the aspects, wherein the hydrogenation catalyst is Rh or a Rh alloy.

Aspect 7. The method of any of the aspects, wherein the hydrogenation catalyst is in nanoparticle form or liquid form; or wherein the hydrogenation catalyst is a solid heterogeneous catalyst contained in a temperature-controlled continuous-flow packed bed reactor.

Aspect 8. The method of any of the aspects, wherein the precursor molecule comprises an unsaturated ester of a C₁ to Cₙ carboxylic acid, where n can be 2 to 20 or n can be 2 to 10 or n can be 2 to 4.

Aspect 9. The method of any of the aspects, wherein the unsaturated ester comprises a vinyl ester or a propargyl ester.

Aspect 10. The method of any of the aspects, wherein the precursor molecule comprises vinyl acetate, propargyl pyruvate, or any combination thereof.

Aspect 11. The method of any of the aspects, wherein the at least one heteronucleus of the precursor molecule is a carbon-13, nitrogen-15, phosphorous-31, or fluorine-19 atom.

Aspect 12. The method of any of the aspects, wherein at least one heteronucleus of the precursor molecule comprises a carbon atom double bonded to an oxygen atom.

Aspect 13. The method of any of the aspects, wherein the precursor molecule further comprises at least one deuterium.

Aspect 14. The method of any of the aspects, further comprising hydrolyzing the ester after hydrogenating the precursor molecule.

Aspect 15. The method of any of the aspects, wherein the spin is transferred from the first parahydrogen atom or the second parahydrogen atom to the third hydrogen atom using adiabatic passage.

Aspect 16. The method of any of the aspects, wherein adiabatic passage is accomplished via exposing the precursor molecule to a detection magnetic field following the hydrogenating, wherein the detection magnetic field has a strength greater than about 2 T.

Aspect 17. The method of any of the aspects, wherein the spin is transferred from the third hydrogen atom to at least one heteronucleus using the insensitive nuclei enhanced by polarization transfer (INEPT) pulse sequence or MINERVA pulse sequence.

Aspect 18. The method of any of the aspects, wherein the method is theoretically at least 80% efficient.

Aspect 19. The method of any of the aspects, wherein the method enhances a magnetic resonance signal for at least one heteronucleus of at least a factor of 100,000.

Aspect 20 A precursor molecule comprising at least one hyperpolarized heteronucleus produced by the method of any of the aspects.

Aspect 21. A contrast agent comprising the precursor molecule of any of the aspects.

Aspect 22. The contrast agent of any of the aspects, wherein the contrast agent is a magnetic resonance imaging contrast agent.

Aspect 23. A system for producing a fluid sample comprising a hyperpolarized heteronucleus, comprising: a precursor solution introduction device, wherein the precursor solution comprises a target substrate molecule and a catalyst; a reaction chamber, wherein the precursor solution introduction device is configured to be in fluid communication with the reaction chamber; a gas introduction system in communication with the reaction chamber, wherein the gas introduction system is configured to introduce parahydrogen into the reaction chamber, wherein the system is configured to contact the precursor solution with the parahydrogen, resulting in the formation of a hyperpolarized fluid sample; an adiabatic transport tube, wherein the adiabatic transport tube is configured to receive the hyperpolarized fluid sample from the reaction chamber and wherein hyperpolarized fluid sample is subjected to an increase in magnetic field, resulting in a transfer of hyperpolarization from at least one parahydrogen atom in the target substrate to a second hydrogen atom in the target substrate; and a sample chamber in fluid communication with the adiabatic transport tube, wherein the hyperpolarized fluid sample is subjected to a high magnetic field, resulting in transfer of hyperpolarization from the second hydrogen to a heteronucleus and generating a target molecule comprising the hyperpolarized heteronucleus.

Aspect 24. The system of any of the aspects, wherein the reaction chamber comprises an ultrasonic nozzle, and wherein the precursor solution introduction device is configured to deliver the precursor solution to the surface of the ultrasonic nozzle, wherein the ultrasonic nozzle is configured to produce droplets of the precursor solution.

Aspect 25. The system of any of the aspects, wherein the droplets of the precursor solution have an average diameter of from about 1 to about 50 μm in a droplet distribution range of about 25 μm or less.

Aspect 26. The system of any of the aspects, wherein the precursor solution introduction device comprises a syringe pump configured to deliver an amount of the precursor solution to the surface of the ultrasonic nozzle.

Aspect 27. The system of any of the aspects, further comprising a detection device, wherein the sample chamber containing the fluid sample comprising a hyperpolarized heteronucleus is configured to fit into the detection device.

Aspect 28. The system any of the aspects, wherein the detection device is a nuclear magnetic resonance spectrometer or a magnetic resonance imaging device.

Aspect 29. The system of any of the aspects, wherein the fluid sample is a homogeneous fluid including the target substrate molecule and a catalyst.

Aspect 30. The system of any of the aspects, wherein the fluid sample is a heterogeneous fluid including the target substrate molecule and a particle comprising a catalyst.

Aspect 31. The system of any of the aspects, wherein the catalyst comprises a Group VIII, IB, or IB transition metal-based catalyst including at least two different metals.

Aspect 32. The system of any of the aspects, wherein the catalyst does not include a metal.

Aspect 33. The system of any of the aspects, wherein the catalyst comprises at least one of Pt, Pd, Cu, Au, Ag, Rh, Ru, Ir, Ni, Sn, Co, Zn, Ce, Ti, Al, Fe, Si, O, or any combination thereof.

Aspect 34. The system of any of the aspects, wherein the catalyst comprises nanoparticle having a diameter of from about 10 to 500 nm.

Aspect 35. The system of any of the aspects, wherein the catalyst is in a liquid form.

Aspect 36. The system of any of the aspects, wherein the catalyst is Rh or a Rh alloy, and wherein the catalyst is supported, tethered, a ligand stabilized in solution, or any combination thereof.

Aspect 37. The system of any of the aspects, wherein the target substrate molecule comprises a metabolite or derivative thereof.

Aspect 38. The system of any of the aspects, wherein the metabolite or derivative thereof can be administered in vitro or to a subject in vivo.

Aspect 39. The system of any of the aspects, wherein the metabolite or derivative thereof comprises an unsaturated ester of a C₁ to C₄ carboxylic acid.

Aspect 40. The system of any of the aspects, wherein the unsaturated ester comprises a vinyl ester or a propargyl ester.

Aspect 41. The system of any of the aspects, wherein the metabolite or derivative thereof comprises vinyl acetate, propargyl pyruvate, or any combination thereof.

Aspect 42. The system of any of the aspects, wherein the at least one heteronucleus of the precursor molecule is a carbon-13, nitrogen-15, phosphorous-31, or fluorine-19 atom.

Aspect 43. The system of any of the aspects, wherein the precursor solution introduction device is configured to introduce precursor solution over a range of speeds, wherein a specific introduction speed can be selected prior to introducing the precursor solution, and wherein the introduction speed of the precursor solution is correlated to an amount of hyperpolarization in the fluid sample.

Aspect 44. A fluid sample comprising a hyperpolarized heteronucleus produced by the system of any of the aspects.

Aspect 45. A method for detecting a disease state associated with abnormal concentration or abnormal activity of a precursor molecule metabolite in a subject, the method comprising: (a) administering the contrast agent of any of the aspects or the fluid sample any of the aspects to the subject; and (b) detecting the contrast agent or the target molecule in the subject.

Aspect 46. The method of any of the aspects, further comprising administering one or more additional contrast agents to the subject, wherein the one or more additional contrast agents can be administered sequentially or simultaneously with the contrast agent or target molecule.

Aspect 47. The method of any of the aspects, wherein the detecting is accomplished using magnetic resonance imaging.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a heteronucleus,” “a metabolite,” or “a parahydrogen atom,” include, but are not limited to, mixtures or combinations of two or more such heteronuclei, metabolites, or parahydrogen atoms, and the like.

The term “heteronucleus” refers to any atomic nucleus other than the proton; e.g., ¹³C, ¹⁵N, ³¹p, ¹⁹F, ²H, or ²⁹Si.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

The term “polarization” refers to the difference in fractional populations of two spin states (for example, the spin-up and spin-down quantum states of the proton, denoted |↑ [symbol] and |↓ [symbol] ). Polarization of a spin-1/2 particle is defined as:

$$P ≡ \frac{{N}_{↑} - {N}_{↓}}{{N}_{↑} + {N}_{↓}} ,$$

where N_{↑} and N_{↓} are the numbers of particles in the spin-up and spin-down states, respectively.

| | | | | | |
|---|---|---|---|---|---|
| [symbol] | \|↓ [symbol] | [symbol] | \|↓ [symbol] | [symbol] | \|↓ [symbol] |
| [symbol] | \|↑ [symbol] | [symbol] | \|↑ [symbol] | [symbol] | \|↑ [symbol] |

| | | |
|---|---|---|
| P = 0 | P = +1 | P = −1 |
| Unpolarized | Fully | Fully |
| | polarized | negatively |
| | | polarized |

The term “hyperpolarization” refers to a non-equilibrium nuclear spin polarization that is enhanced relative to the Boltzmann thermal equilibrium spin polarization.

The phrase “hyperpolarized fluid sample” refers to a liquid or gas containing target molecules hosting hyperpolarized nuclear spins.

The phrase “precursor solution” refers to a liquid solvent or solvent mixture containing a catalyst complex or catalyst nanoparticles together with the hydrogenation substrate molecules that incorporate at least one unsaturated chemical moiety (e.g. a double bond, a triple bond, a carbonyl group, or a hydroxy group) to which one or more protons of H₂ may be transferred, or alternatively, that becomes hyperpolarized through a spin order transfer mechanism that does not involve chemical transfer of any protons, e.g. by Signal Amplification by Reversible Exchange (SABRE), Surface Waters are Magnetized from Parahydrogen (SWAMP), or Nuclear Exchange Polarization by Transposing Unattached Nuclei (NEPTUN).

The term “parahydrogen” refers to the metastable spin isomer of dihydrogen with proton spins in a singlet state that is antisymmetric with respect to permutation of the two protons. For simplicity, the term parahydrogen will in some cases, depending on the context, also refer to dihydrogen gas that is only partially enriched in the parahydrogen spin isomer content relative to normal hydrogen, which is about 25% parahydrogen and about 75% orthohydrogen (the triplet state, which is symmetric with respect to permutation of the two protons).

The term “heteronuclei” will refer to the spin-1/2 isotopes other than the proton, including carbon-13, nitrogen-15, fluorine-19, and phosphorus-31.

“INEPT” or “insensitive nuclei enhancement by polarization transfer” refers to a signal enhancement method used in nuclear magnetic resonance spectroscopy and magnetic resonance imaging. An INEPT pulse sequence can be used to transfer nuclear spin polarization from protons, including, but not limited to, parahydrogen atoms, to a heteronucleus.

“MINERVA” or “(Maximizing Insensitive Nuclear Enhancement Reached Via para-hydrogen Amplification)” refers to a coherence transfer pulse sequence for converting bilinear spin order generated by the PASADENA, ALTADENA, or LACADENA effects into hyperpolarized magnetization of a heteronucleus.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Parahydrogen based hyperpolarization is an efficient and inexpensive method for sensitivity-enhanced nuclear magnetic resonance (NMR) spectroscopy and magnetic resonance imaging (MRI). Conversion of parahydrogen (p-H₂) singlet spin order into NMR observable nuclear spin hyperpolarization is mediated by chemical hydrogenation into magnetically inequivalent sites, resulting in high-field, room temperature NMR signal enhancements that can in theory exceed four orders of magnitude for protons and five orders of magnitude for carbon-13. An essential requirement of parahydrogen-based hyperpolarization is pairwise addition, where the pair of H atoms that are incorporated into the hydrogenation adduct originate from the same p-H₂ molecule, thereby preserving the singlet spin order. Many important biomolecules and metabolites, such as pyruvate and acetate, are not directly producible by addition of dihydrogen, and this is a practical limitation on the scope of the parahydrogen technique. Side-arm hydrogenation (SAH) provides one solution to this problem. A carboxylic acid is esterified with a short side-arm moiety containing a C≡C or C═C bond (e.g., propargyl, allyl, or vinyl alcohol). After hydrogenating the side-arm with p-H₂, the parahydrogen spin order is transferred via J-couplings across the ester linkage to the carbonyl ¹³C. In the final step, the side-arm is cleaved to release the hyperpolarized pyruvate or acetate into the aqueous phase. There are currently two approaches to spin order transfer (SOT): (J) ultra-low field magnetic field cycling (MFC), which brings the ¹³C and ¹H nuclei into strong coupling at sub-microTesla magnetic fields, and (ii) coherence transfer radio frequency pulse sequences (e.g., sINEPT, INEPT, relayed-INEPT, and MINERVA) which are applied following hydrogenation with parahydrogen in the high magnetic field of the NMR spectrometer. The goal of SOT methods is to move p-H₂ spin order to Zeeman hyperpolarization of a heteronucleus as efficiently as possible.

In this disclosure, a new two-step spin SOT method is disclosed. Firstly, hydrogenation of unsaturated substrate molecules with parahydrogen is performed at low magnetic field in the strong proton-proton coupling regime. Secondly, the product molecules are adiabatically transported to high magnetic field where the proton pair is weakly coupled. In the second step, the proton spin hyperpolarization originating from parahydrogen is transferred to the heteronucleus using a using one of the aforementioned coherence transfer pulse sequences. Importantly, this two-step process is equally applicable to two types of hydrogenation adducts. In the first, there is a significant spin-spin coupling between the heteronucleus and at least one of the protons originating from parahydrogen. This is the case, for example, for the ester carbonyl carbon of pyruvate (or acetate) in ethyl pyruvate (acetate) formed by hydrogenation of vinyl pyruvate (acetate), respectively. In this case, an INEPT, selective INEPT or MINERVA coherence transfer pulse sequence is applied to induce transfer of spin polarization to the heteronucleus with a very high efficiency approaching 100%. In the second case, the selective INEPT pulse sequence is applied to a third proton (or set of protons) that did not originate from p-H₂. Examples include allyl acetate and allyl pyruvate formed by hydrogenation of propargyl acetate or propargyl pyruvate with parahydrogen. It will be shown that in these examples, parahydrogen spin order is spontaneously shuttled to the methylene proton(s) due to adiabatic passage through level-anticrossings (LACs) involving the parahydrogen protons and the methylene proton(s). Therefore, coherence transfer of parahydrogen spin order to the heteronucleus requires only a single coherence transfer step. The overall efficiency of this LAC-mediated process can be very high. In the specific example of allyl acetate or allyl pyruvate, numerical density matrix simulations reveal a theoretical efficiency of >10%.

Avoided crossings of eigenvalue curves, also known as level anti-crossings (LACs), are ubiquitous in quantum systems and underpin diverse quantum phenomena. Examples include quantum information storage in diamond NV centers, tunneling effects in molecular magnets, chemically induced dynamic nuclear polarization, dynamic nuclear polarization magic angle spinning nuclear magnetic resonance (NMR), and spin order transfer (SOT) in systems of coupled nuclear spins. In pairs of spins with near magnetic equivalence, LACs induced by resonant RF fields can interconvert singlet and triplet spin order. In the realm of parahydrogen-based hyperpolarization, LACS induced by heteronuclear spin-spin coupling have been exploited to convert the bilinear spin order of parahydrogen (pH₂) into hyperpolarization of heteronuclei such as ¹³C or ¹⁵N. Proton-proton spin couplings can also give rise to LACS, as shown by Ivanov et al. In this disclosure, it will be shown that adiabatic passage through LACS arising from certain combinations of homonuclear proton-proton couplings and Zeeman interactions can mediate spontaneous conversion of parahydrogen bilinear spin order of into hyperpolarization (i.e., enhanced, non-equilibrium nuclear spin magnetization) of a third initially unpolarized proton after hydrogenation with parahydrogen. The Zeeman order of the third proton can be subsequently converted to Zeeman order on a heteronucleus (i.e., an isotope other than proton, including carbon-13, nitrogen-15, phosphorous-31, or fluorine-19).

In the ALTADENA (adiabatic transport after dissociation engenders net alignment) effect, a precursor molecule is hydrogenated with p-H₂ at a low magnetic field where the protons are strongly coupled and the eigenfunctions are linear combinations of direct product states. The adduct (i.e, the product of the hydrogenation reaction) is subsequently transported under adiabatic passage conditions to high magnetic field, where the proton spin eigenfunction approximated as the eigenfunctions of the weak-coupling Hamiltonian. For a two proton adduct, where both protons originated from the same parahydrogen, the density operator of the proton pair, is described by

$$ρ = \frac{1}{4} - f {I}_{1} \cdot {I}_{2}$$

where

$$f = \frac{1}{3} ( 4 {x}_{p} - 1 )$$

for an arbitrary p-H₂ mole fraction x_{P}. After adiabatic transport to high magnetic field, the final density operator, in the absence of other protons that did not originate from p-H₂, is

$$ρ = \frac{1}{4} - f [ {I}_{z 1} \cdot {I}_{z 2} ∓ \frac{1}{2} ( {I}_{z 1} - {I}_{z 2} ) ]$$

The 2^{nd} term corresponds to “net alignment” spin order. This net-alignment Zeeman spin order can be subsequently transferred to a heteronucleus in a single step by a selective or nonselective INEPT (insensitive nuclei enhancement by polarization transfer) coherence transfer pulse sequence, which in principle can achieve near 100% efficiency of polarization transfer.

Several methods for producing pyruvate and other metabolites containing hyperpolarized ¹³C are known, such as ultra-low magnetic field cycling and high field coherence techniques. These existing methods suffer from one or more shortcomings. Magnetic field cycling suffers from low efficiency of spin order transfer, and require the use of magnetic shielding to attain sub-microTesla magnetic field. Methods where hydrogenation and spin order transfer entirely at high magnetic also suffer from low incomplete spin order transfer, or losses during the application of complex pulses sequences. Furthermore, the limited space within the bore of a high-field NMR magnet restricts the design of the spray-injection hydrogenation reactor.

Embodiments of the present disclosure provide for ATLADENA-sINEPT and ALTADENA-MINERVA methods for spin order transfer (SOT) in vinyl ester side-arm hydrogenation with parahydrogen. The theoretical efficiency of this method is 97% and is the same even if both of the parahydrogen-sourced protons have non-negligible couplings to the carbon-13 heteronucleus. The efficiency is higher and more robust than other methods that appeared in the literature. In addition, a second type of process, referred to as LACADENA-sINEPT and LACADENA-MINERVA, is applicable to propargyl or similar esters where the carbon-13 heteronucleus is separated from the parahydrogen protons by an additional carbon atom. The shuttling of parahydrogen spin order is mediated by adiabatic passage through level-anticrossings mediated by proton-proton spin coupling. The carbon-13 polarization achieved in this process can be increased by selective deuteration of the side-arm moiety. The deuteration eliminates sharing of parahydrogen spin order to ancillary protons. Non-adiabaticity effects can lower the shuttling efficiency. Furthermore, the present disclosure provides for an ultrasonic liquid shuttling system. This system can facilitate scaling-up of hyperpolarized metabolite production and has the additional advantages of compatibility with flow-chemistry and continuous-flow production of hyperpolarized metabolites and other molecules. Additional aspects include single deuteration at the CH₂ group of the propargyl side arm, incorporation of multiple chemical shift refocusing pulses into the sINEPT pulse sequence, faster experimental transfer, with constant-adiabaticity transport through LACS.

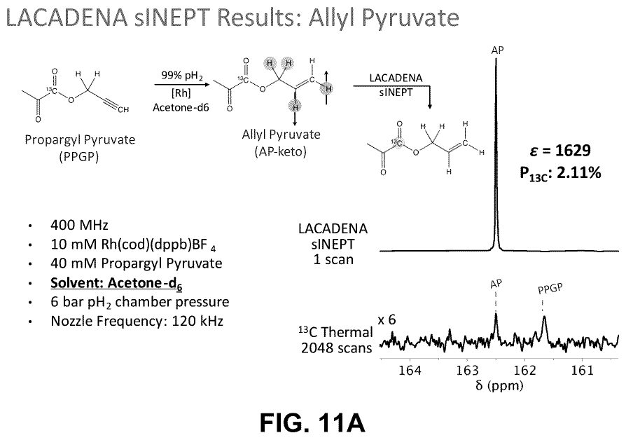

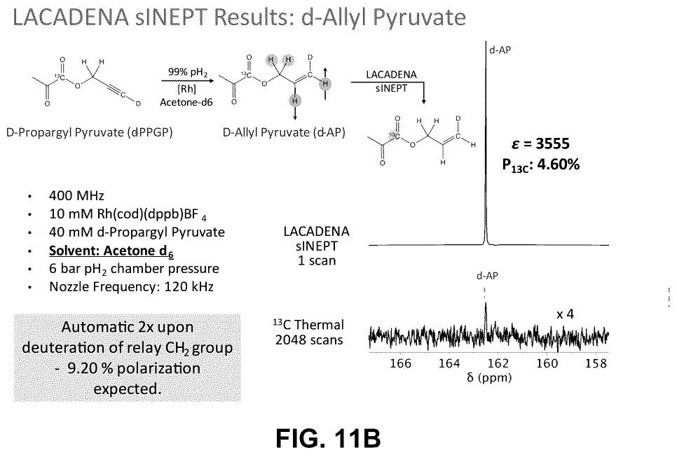

FIG. 11 illustrates experiment results for the LACADENA-sINEPT experiment performed with Propargyl Pyruvate (FIG. 11A) and d-Propargyl Pyruvate (FIG. 6A-C, FIG. 11B), respectively. The carbon-13 NMR signal enhancements of 1629 and 3555 were observed, respectively, corresponding to absolute carbon-13 polarization levels of 2.11% and 4.60%, respectively. These results confirm the significant improvement in the carbon-13 polarization that results from deuteration of the propargyl CH group. Furthermore, the theoretical simulations indicate an additional factor of two will be gained by single deuteration of the CH₂ group of the propargyl side-arm.

### Example 1: Adiabatic Passage Through Level Anti-Crossings Mediates Highly Efficient Relayed Bilinear Spin Order Conversion to Zeeman Polarization in Coupled Systems of Three or Four Protons

This effect will be shown to be broadly operative in a range of hydrogenation adducts, including certain unsaturated propargyl esters of pyruvate and acetate, important metabolites at the intersection of many biochemical reactions. This has important practical implications, because it provides an ideal initial condition for the subsequent coherence transfer of the Zeeman hyperpolarization to the carbonyl ¹³C on the other side of the ester linkage can. Depending on the details of the spin system, the overall SOT process, starting with bilinear proton spin order derived from pH₂, can provide a new highly efficient route for preparing ¹³C hyperpolarized pyruvate and other metabolites that are suitable for sensitivity-enhanced magnetic resonance imaging of disease processes at the cellular level.

Pyruvate and acetate are not directly producible by hydrogenation reactions, and this is a practical limitation on the scope of the parahydrogen technique. Side-arm hydrogenation (SAH) provides a clever work-around to this problem. The carboxylic acid is esterified with a short side-arm moiety containing a C≡C or C═C bond (e.g., propargyl, allyl, vinyl alcohol). Shown in FIGS. 1A-11B and FIG. 2 show the molecular structures of several side-arm modified pyruvate and acetate esters. After hydrogenating the side-arm with pH₂, the bilinear parahydrogen spin order must be transferred across the ester linkage via the J-couplings to the carbonyl ¹³C. In the final step, the side-arm is cleaved to release the hyperpolarized pyruvate or acetate. There are currently two approaches to spin order transfer (SOT): (i) magnetic field cycling (MFC), which brings the ¹³C and ¹H nuclei into the strong coupling regime at sub-μT magnetic fields, and (ii) modified INEPT RF pulse sequences which are applied following hydrogenation in the high magnetic field of the NMR spectrometer. The goal of SOT methods is to move the pH₂ bilinear spin order involving H¹ and H² to Zeeman hyperpolarization of ¹³C⁴ as efficiently as possible. The initially unpolarized H^{3,6} nuclei are referred to as the relay protons. They can be used to relay spin order because the direct couplings J₁₄ and J₂₄ are too small to mediate efficient SOT to the ¹³C⁴ in a single step. Note that the APd2 and BPd2 differ only by a —CD₃ group on the relay carbon. Remarkably, it will be shown that this minor chemical modification in BPd2 completely shuts-off the spontaneous LAC-mediated shuttling of parahydrogen spin order from H^{1,2} to H³. A vinyl ester precursor in which the relay protons are absent is also shown in FIG. 1A. While this precursor affords direct SOT in a single step, the rate of hydrogenation of the vinyl group is significantly slower than the rate for the propargyl hydrogenation. The experimental study revealed that propargyl pyruvate (acetate) is advantageous in achieving the highest absolute polarizations in both INEPT and MFC methods. The PASADENA mode proton-relay experiment in BP-d4 exhibited an absolute ¹³C polarization of 7% starting from an initial 14% Î_{1z}Î_{2z} bilinear proton spin order.

To elucidate the spin Hamiltonian parameters required for spontaneous SOT to H³ during adiabatic passage, consider isotropic lab-frame Hamiltonians (ℏ=1) of the form

$$\hat{H} ( t ) = {\sum }_{i < j}^{3} {J}_{12} {\hat{I}}_{i} + {\hat{I}}_{j} + {\sum }_{i = 1}^{3} γ B ( t ) ( 1 - {δ}_{i} ) {\hat{I}}_{iz} .$$

Here, γ is the proton gyromagnetic ratio, B is the applied magnetic field, δᵢ the chemical shift of the i^{th} proton, Î_{iz} is the z-component of the i^{th} spin operator Îᵢ, and JᵢⱼÎᵢ·Îⱼ is the scalar spin-spin coupling between protons Hⁱ and H^{j}. Using the labelling scheme of FIG. 1, protons H¹ and H² originate from parahydrogen while H³ is the pre-existing unpolarized relay proton. The discussion will initially focus on idealized model Hamiltonians where J₁₃=0; hence, H¹ is coupled H² and H² to H³, but H¹ and H³ are not directly coupled. Depending on the relative strengths of the Zeeman interactions of the three protons, a LAC at a finite magnetic field may or may not occur. FIG. 7 presents rotating frame eigenvalue correlation diagrams for three idealized constructs: C1, δ₁>δ₂>δ₃; C2, δ₁>δ₃>δ₁; C3, δ₁=δ₂>δ₃. Replacing ‘>’ by ‘<’ generates three addition constructs with analogous spin physics. The chemical shifts and couplings will be assumed to fulfill weak coupling at the end of the field ramp, where γB₀|δ₂−δ₃|>>|J₂₃| and γB₀|δ₁−δ₂|>>|J₁₂| (when δ₁≠δ₂), an assumption that is valid for both AP and BP. The conservation of parahydrogen spin order through the hydrogenation reaction is maximal when |J₁₂|>>|J₂₃|, which ensures that upon introduction of J₂₃, the parahydrogen singlet state |S₀ [symbol] =2^{-1/2}|αβ−βα) remains largely intact after projection onto the adduct eigenstates.

Eigenvalue plots generated for J₂₃=0 are also shown in the bottom diagrams of FIGS. 7A-7B. Notably, LACs occur only for C1 and C3, where δ₃ lies outside of the range (either higher or lower) spanned by δ₁ and δ₂. A LAC does not occur for BP, a C2 molecule, where δ₃ is intermediate between δ₁ and δ₂.

Qualitatively, the final population distribution over the high-field eigenstates can be inferred from the eigenvalue correlation plots. For quantitative analysis, the Zeeman order (i.e., polarization÷2) of each proton at the end of the adiabatic sweep may be calculated from [symbol] =Tr( [symbol] · [symbol] ). The density operator at the terminus of field ramp may be calculated (neglecting spin relaxation) from the Liouville-von Neumann (LVN) equation using Ĥ(t).

$$\frac{\partial }{\partial t} \hat{ρ} = i [ \hat{ρ} , \hat{H} ]$$

$$\hat{ρ} ( t ) = exp ( - i {{\int }_{0}}^{t} {dt}^{′} \hat{H} ( {t}^{′} ) ) \hat{ρ} ( 0 ) exp ( + i {{\int }_{0}}^{t} {dt}^{′} \hat{H} ( {t}^{′} ) )$$

where \hat{T} is the Dyson time-ordering operator. In adiabatic passage, B(t) is varied smoothly from zero (or low) field (where hydrogenation is performed) to the terminal high field of the ramp where the “hyperpolarized” NMR spectrum is acquired.

In adiabatic passage, B(t) is varied smoothly from zero (or low) field (where hydrogenation is performed) to the terminal high-field of the ramp where the “hyperpolarized” NMR spectrum is acquired.

The density operator at the moment of pH₂ addition is:

$${\hat{ρ}}_{s} = \frac{1}{4} ( \vert {S}_{0} α 〉 〈 {S}_{0} α \vert + \vert {S}_{0} β 〉 〈 {S}_{0} β \vert ) = ( \frac{\hat{1}}{4} - {\hat{I}}_{1} \cdot {\hat{I}}_{2} ) ⊗ \frac{1}{2} \hat{1}$$

Since [Ĥ, \hat{ρ}]≠0, sudden introduction of the J₂₃ interaction induces transitions between eigenstates. Assuming a broad kinetic distribution of addition times, time evolution of individual adduct spin states will generally result in loss of coherence (off-diagonal elements of the matrix representation of the density operator in the eigenbasis). Let [symbol] \hat{ρ}ₛ [symbol] denote the time-averaged density operator. To the extent that |J₁₂|>>|J₂₃|, the diagonal elements in the matrix representation of \hat{ρ} will be preserved for hydrogenation at low/zero magnetic fields; hence \hat{ρ}ₛ= [symbol] \hat{ρ}ₛ [symbol] , as already noted. Losses due to projection onto eigenstates that deviate from the singlet/triplet basis will reduce the overall theoretical efficiency of conversion of singlet order to observable Zeeman hyperpolarization.

Following hydrogenation, the magnetic field is then immediately ramped up to the detection field:

$$〈 {\hat{ρ}}_{s} 〉 {\to }^{\hat{H} ( t ) , adiabatic} {\hat{ρ}}_{1}$$

In adiabatic passage, where the duration of the ramp is short compared to the spin-lattice relaxation time yet slow enough to not induce transitions, the occupancy of each eigenstate is maintained throughout the ramp. The specific form of B(t) is immaterial so long as the adiabatic criterion is obeyed at all times, especially in the vicinity of LACs.

Results for the idealized model Hamiltonians are summarized in Table 1. For C3, the singlet remains an eigenstate throughout the ramp to high field, and an NMR signal is obtained only if J₂₃>0. A 1:−1 net alignment intensity pattern is predicted, corresponding to [symbol] I_{1z} [symbol] = [symbol] I_{1z} [symbol] =¼ and [symbol] I_{3z} [symbol] =−½. For C1 and C2 with J₂₃=0, the classic ALTADENA effect is evident with H³ remaining an unpolarized spectator. For J₂₃>0, the initially unpolarized H³ becomes fully polarized at the expense of H¹. In C2, where the Zeeman energy of H³ is intermediate to H¹ and H², there is no LAC and hence no shuttling of spin order to H³, so it remains an unpolarized spectator irrespective of J₂₃. These results imply that there is no proton-proton induced LAC in BPd4, and therefore, the LAC-ALTADENA INEPT approach will be ineffective for this choice of side-arm.

TABLE 1

| Idealized Model | | | | | | | | | | |
|---|---|---|---|---|---|---|---|---|---|---|
| | Hamiltonian | | | | | | | | | |
| Chemical | J₂₃ = 0 | | | J₂₃ > 0 | | | Embodiment (simulation) | | | |
| Shifts | [symbol] I_{1z} [symbol] | [symbol] I_{2z} [symbol] | [symbol] I_{3z} [symbol] | [symbol] I_{1z} [symbol] | [symbol] I_{2z} [symbol] | [symbol] I_{3z} [symbol] | Molecule | [symbol] I_{1z} [symbol] | [symbol] I_{2z} [symbol] | [symbol] I_{3z} [symbol] |
| C1: δ₂ > δ₁ > δ₃ | +1/2 | −1/2 | 0 | 0 | +1/2 | −1/2 | AP | +.27 | −.15 | −.12^{a} |
| | | | | | | | AA | +.27* | −.14* | −.13*^{,a} |
| | | | | | | | APd2 | +.46 | −.046 | −.41 |
| | | | | | | | AAd2 | | | |
| C2: δ₂ > δ₃ > δ₁ | +1/2 | −1/2 | 0 | +1/2 | −1/2 | 0 | BPd4 | +.46 | −.46 | 2.4 × 10⁻³ |
| C3: δ₁ = δ₂ > δ₃ | 0 | 0 | 0 | +1/4 | +1/4 | −1/2 | FA | −.25 | −.25 | +.50^{b} |
| | 0 | 0 | 0 | +1/4 | +1/4 | −1/2 | CB | −.20 | −.20 | +.40^{c} |

*Peak integrals from experimental ALTADENA AA spectrum (non-deuterated).
^{a}Σ [symbol] I_{z} [symbol] over both methylene relay protons (H² and H⁶).
^{b}Σ [symbol] I_{z} [symbol] over two hydroxy protons.
^{c}Σ [symbol] I_{z} [symbol] over six methyl protons.

Remarkably, the idealized constructs C1 and C3 illustrate how adiabatic passage through a LAC can spontaneously shuttle spin order to an initially unpolarized third proton H³ not originating in pH₂. The model Hamiltonian parameters, with J₁₂>>J₂₃, gave near-perfect efficiency in this process. Now, the actual side-arm modified metabolite molecules AP and BP will be considered. For these real molecules, the spin Hamiltonians are less than ideal but still favor efficient SOT. The ordering of chemical shifts in AP (or allyl acetate, AA) is that of C1, while BP is a C2 system and is thus predicted not to exhibit a LAC. In the non-deuterated AP (or AA or BP), the J couplings involving H⁵ result in an irreversible and significant loss of pH₂-derived spin order upon hydrogenation. This is because J₂₅ is large compared to J₁₂ and J₂₃, and so there is a large admixture of the H⁵ spin-state in the low field eigenstates. The destructive effect of J₂₅ in the non-deuterated form is evident by evaluating Tr{\hat{ρ}ₛ· [symbol] \hat{ρ}ₛ [symbol] )}. For a pure state, Tr{\hat{ρ}²}=1, but since H³ and H⁵ are initially unpolarized, Tr{\hat{p}²}=¼ maximally. If J₁₅→0 in AP, one obtains Tr{\hat{ρ}ₛ· [symbol] \hat{ρ}ₛ [symbol] )}=0.21, but when J₁₅=16.6 Hz, it is reduced to Tr{\hat{ρ}ₛ· [symbol] \hat{ρ}ₛ [symbol] }=0.122. The loss can be mitigated by selective deuteration, since J_{HD}/J_{HH}≈ 1/7. Furthermore, if hydrogenation is performed in a small but finite magnetic field (e.g., Earth's field) where deuterons and protons are weakly coupled, state mixing is negligible. This is confirmed by the numerical simulation results in Table 1, showing that deuteration @H⁵ results in an approximate three-fold increase of [symbol] I_{3z} [symbol] .

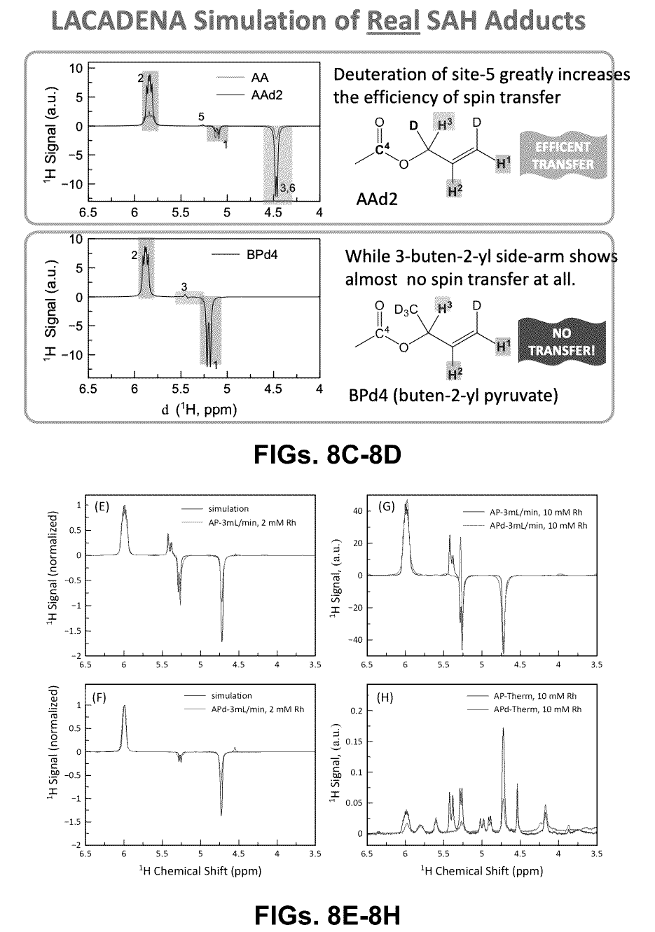

The numerically simulated ALTADENA spectrum of AA (non-deuterated), Hilbert space simulation in Matlab/Spinach, is overlaid on the experimental spectrum in FIG. 8A. Using the measured axial dependence of the magnetic field of the 7 T Oxford magnet and a transport time of 2 s (assuming constant velocity) in the numerical simulations, good agreement was obtained. The chemical shifts and J-couplings were deduced from the thermally polarized experimental spectrum, shown together with its numerical simulation in FIG. 8B. These results confirm the interpretation of the eigenvalue correlation diagrams, validate the disclosed numerical methods, and demonstrate that the experimentally employed field ramp is to a good approximation adiabatic. The spectra of the deuterated allyl and 3-buten-2-yl esters are presented in FIGS. 8C-8D. Confirming the results of Table 1, the signal enhancement of the H³ multiplet is significantly improved by the selective deuteration @H⁵ in AAd2, while for BPd4, where there is no LAC, the H³ signal is extremely weak.

The 400 MHz ¹H ALTADENA NMR spectra of AP and APd acquired after ultrasonic spray injection of the propargyl pyruvate precursor solutions containing 2 mM Rh catalyst into the reaction chamber pressurized with 99% pH₂ followed by infusion into the NMR flow probe at 3 mL/min (τₜᵣ=6.6 s) are presented in FIG. 8E and FIG. 8F. Superimposed on these spectra are the corresponding numerical simulations based on the measured magnetic field profile of the 9.4 T Bruker Ultrashield magnet. Each spectrum has been normalized to its H² peak amplitude. Without any fitting parameters, excellent agreement between theory and experiment is obtained except for the H³ resonance, which is noticeably lower in intensity than in the simulated spectra of both AP and APd. The discrepancy is attributed to the shorter spin-lattice relaxation time of the H³ methylene protons (see Supporting Information). The effects of differential spin-lattice relaxation losses during transport become even more pronounced at lower flow rates (see Supporting Information). FIG. 8G presents the spectrum obtained using the same flow rate of 3 mL/min but with a 5× more concentrated precursor solution (10 mM Rh and 40 mM AP or APd). For APd, a LAC mediated polarization of P₃=19.8±2.6% was obtained for the initially unpolarized relay (methylene) protons, while a remarkable P₂=68.7±0.5% polarization (averaged over 3 trials) was obtained for the vinylic H² proton originating from pH₂. Interestingly, the H¹ signal in the spectrum of APd obtained using the more concentrated precursor solution exhibits a clear antiphase doublet that defied numerical simulation. This contrasts with the net emission doublet observed with the APd precursor solution of lower concentration (FIG. 8F), consistent with theory. Signal enhancement and polarization values were calculated using the signals in the thermally polarized spectra shown in FIG. 8H and are reported in the table below.

| | Average Measured Enhancements | | | Average Measured Polarizations | | |
|---|---|---|---|---|---|---|
| | H¹ | H² | H³ | H¹ | H² | H³ |
| AP | 1926 ± 369 | 4405 ± 338 | 1020 ± 89 | 9.9 ± 1.9% | 22.7 ± 1.7% | 5.3 ± 0.5% |
| APd | 1744 ± 159 | 13350 ± 90 | 3852 ± 504 | 9.0 ± 0.8% | 68.7 ± 0.5% | 19.8 ± 2.6% |

Lastly, presented herein are the calculations for two C3 embodiments where exact chemical equivalence of H¹ and H² implies a symmetric molecular structure. In fumaric acid, a molecule of interest for imaging of tissue necrosis, J̅₁₂=15.7 Hz and J̅₁₃=J̅₂₄<1 Hz, while J̅₁₄ and J̅₂₄ are negligible; hence this spin Hamiltonian is near ideal. Indeed, numerical simulations predict a hydroxyl proton Zeeman order of [symbol] I_{3z} [symbol] + [symbol] I_{4z} [symbol] =0.50, within 1% of the theoretical maximum. Cis-2-butene, where the individual H³ and H protons of C3 are replaced with —CH₃ groups with an inter-methyl proton coupling of 1.18 Hz and J̅₁₃=J̅₂₄˜−1.79 Hz, is less than ideal, yet still exhibits high efficiency LAC relayed transfer with a final total methyl spin polarization of Σᵢ₌₃⁸ [symbol] I_{iz} [symbol] =+0.40.

Previous SOT techniques have utilized two-step H^{1,2}→H³→¹³C⁴ or H^{1,2}→¹³C²→¹³C⁴ coherence transfer pathways in PASADENA type experiments, where hydrogenation is performed at high magnetic field. The sequences that proceed through conversion of antiphase two-spin order into in-phase proton spin order, or where single quantum coherence is induced by a 45° pulse are inherently less efficient (by a factor of two) due to kinetic averaging losses of coherence spin order after projection of the pH₂ singlet onto the Zeeman eigenstates, as discussed above, and because of incomplete conversion of the parahydrogen spin order. In previous ALTADENA mode experiments, where hydrogenation is performed at low fields in the strong heteronuclear coupling regime, SOT to ¹³C occurs via spin-state mixing. The SOT is enhanced in the vicinity of level anti-crossings (LACS) arising from ¹³C-¹H coupling. This method is also inherently less efficient than the hybrid ALTADENA-INEPT method because the ¹H spin order is diluted across all coupled protons in the spin system.

In summary, it has been shown that adiabatic passage through a LAC resulting from proton-proton couplings and Zeeman interactions can elicit a complete and spontaneous conversion with simultaneous translation of parahydrogen singlet-spin order to hyperpolarized Zeeman order of third initially unpolarized spin. This approach can in principle produce near unity levels of spin polarization in ¹³C-pyruvate, a key molecule for MRI detection of cancer and other diseases based on the Warburg effect. Following the LAC-mediated SOT to the relay proton H³, a simple, selective INEPT sequence can be applied to transfer the Zeeman spin order to the carbonyl ¹³C with 100% efficiency, so that the overall efficiency is calculated to be 82% for APd2. When 100% pH₂ is used, this would yield a proton signal enhancement factor of 35,200 for relative to signals derived from thermal equilibrium Boltzmann polarization at 7 T (300 MHz), and a factor of 140,000 for the carbonyl ¹³C of APd2. As with any experiment with parahydrogen, the observed enhancement factors are reduced by spin-lattice relaxation during hydrogenation and any time delays prior to spectrum acquisition. The overall theoretical quantum efficiency of this new hybrid route is higher than other SOT approaches. The theoretical maximum polarization attainable by the parahydrogen-based hyperpolarization approach, when combined with side-arm hydrogenation and ALTADENA-INEPT spin order transfer, compares favorably with dissolution dynamical nuclear polarization, but with the important practical advantages: substantially shorter polarization times, higher reproducibility, compatibility with flow chemistry, and lower cost.

### REFERENCES FOR EXAMPLE 1

1. Ardenkjaer-Larsen, J. H. On the Present and Future of Dissolution-DNP. 2016, 264, 3-12.
2. Ardenkjaer-Larsen, J. H.; et al. Increase in Signal-to-Noise Ratio of >10,000 Times in Liquid-State NMR. Proceedings of the National Academy of Sciences of the United States of America 2003, 100 (18), 10158-10163.
3. Bengs, C.; et al. SpinDynamica: Symbolic and Numerical Magnetic Resonance in a Mathematica Environment. Magnetic Resonance in Chemistry 2018, 56 (6), 374-414. https://doi.org/10.1002/MRC.4642.
4. Bowers, C. R.; et al. Parahydrogen and Synthesis Allow Dramatically Enhanced Nuclear Alignment. Journal of the American Chemical Society 1987, 109 (18), 5541-5542.
5. Bowers, C. R. Sensitivity Enhancement Utilizing Parahydrogen. In Encyclopedia of Magnetic Resonance; Grant, D. M., Harris, R. K., Eds.; John Wiley & Sons, Ltd: Chichester, UK, 2007; Vol. 9, p 750.
6. Cavallari, E., et al. Effects of Magnetic Field Cycle on the Polarization Transfer from Parahydrogen to Heteronuclei through Long-Range J-Couplings. Journal of Physical Chemistry B 119, 10035-10041 (2015).
7. Cavallari, E.; et al. Metabolic Studies of Tumor Cells Using [1-13 C] Pyruvate Hyperpolarized by Means of PHIP-Side Arm Hydrogenation. ChemPhysChem 2019, 20 (2), 318-325.
8. Cavallari, E.; et al. Studies to Enhance the Hyperpolarization Level in PHIP-SAH-Produced C13-Pyruvate. Journal of Magnetic Resonance 2018, 289, 12-17.
9. Dagys, L.; et al. Nuclear Hyperpolarization of (1-13C)-Pyruvate in Aqueous Solution by Proton-Relayed Side-Arm Hydrogenation. Analyst 2021, 146 (5), 1772-1778.
10. Eills, J.; et al. Real-Time Nuclear Magnetic Resonance Detection of Fumarase Activity Using Parahydrogen-Hyperpolarized [1-13C]Fumarate. Journal of the American Chemical Society 2019, 141 (51), 20209-20214.
11. Fuchs, G. D. et al. A Quantum Memory Intrinsic to Single Nitrogen-Vacancy Centres in Diamond. Nature Physics 2011 7:10 2011, 7 (10).
12. Heiden, M. G. V.; et al. Understanding the Warburg Effect: The Metabolic Requirements of Cell Proliferation. Science 2009, 324 (5930), 1029-1033.
13. Hogben, H. J.; et a. Spinach—A Software Library for Simulation of Spin Dynamics in Large Spin Systems. Journal of Magnetic Resonance 2011, 208 (2), 179-194.
14. Ivanov, K. L. et al. The Role of Level Anti-Crossings in Nuclear Spin Hyperpolarization. Progress in Nuclear Magnetic Resonance Spectroscopy 2014, 81, 1-36.
15. Joalland, B.; et al. Pulse-Programmable Magnetic Field Sweeping of Parahydrogen-Induced Polarization by Side Arm Hydrogenation. Analytical Chemistry 2020, 92 (1), 1340-1345.
16. Jóhannesson, H.; et al. Transfer of Para-Hydrogen Spin Order into Polarization by Diabatic Field Cycling. Comptes Rendus Physique 2004, 5 (3), 315-324.
17. Knecht, S.; et al. Rapid Hyperpolarization and Purification of the Metabolite Fumarate in Aqueous Solution. Proceedings of the National Academy of Sciences 2021, 118 (13), e2025383118.
18. Korchak, S.; et al. Over 50% 1H and 13C Polarization for Generating Hyperpolarized Metabolites—A Para-Hydrogen Approach. ChemistryOpen 2018, 7 (9), 672-676.
19. Korchak, S., et al. Pulsed Magnetic Resonance to Signal-Enhance Metabolites within Seconds by utilizing para-Hydrogen. ChemistryOpen 7, 344-348 (2018).
20. Pravdivtsev, A. N.; et al. Highly Efficient Polarization of Spin-1/2 Insensitive NMR Nuclei by Adiabatic Passage through Level Anticrossings. The Journal of Physical Chemistry Letters 2014, 5 (19), 3421-3426.
21. Pravica, M. G.; et al. Net NMR Alignment by Adiabatic Transport of Parahydrogen Addition Products to High Magnetic Field. Chemical Physics Letters 1988, 145 (4), 255-258.
22. Reineri, F.; et al. ParaHydrogen Induced Polarization of 13C Carboxylate Resonance in Acetate and Pyruvate. Nature Communications 2015, 6, 1-6.
23. Ripka, B.; et al. Hyperpolarized Fumarate via Parahydrogen. Chemical Communications 2018, 54 (86), 12246-12249.
24. Salnikov, O. G. et al. Parahydrogen-Induced Polarization of 1-13C-Acetates and 1-13C-Pyruvates Using Sidearm Hydrogenation of Vinyl, Allyl, and Propargyl Esters. The Journal of Physical Chemistry C 2019, 123 (20), 12827-12840.
25. Svyatova, A.; et al. PHIP Hyperpolarized [1-13C]Pyruvate and [1-13C]Acetate Esters via PH-INEPT Polarization Transfer Monitored by 13C NMR and MRI. Scientific Reports 2021, 11 (1), 5646.
26. Vogelsberger, M.; et al. Butterfly Hysteresis Curves Generated by Adiabatic Landau-Zener Transitions. Physical Review B 2006, 73 (9), 092412.
27. Zhou, J.; et al. Observation of Time-Domain Rabi Oscillations in the Landau-Zener Regime with a Single Electronic Spin. Physical Review Letters 2014, 112 (1), 010503.
28. Zhukov, I. V. et al. Field-Cycling NMR Experiments in an Ultra-Wide Magnetic Field Range: Relaxation and Coherent Polarization Transfer. Physical Chemistry Chemical Physics 2018, 20 (18), 12396-12405.

### Example 2: Coherence Transfer after Adiabatic Longitudinal Transport: A Versatile Spin Order Transfer Process for Side-Arm Hydrogenation with Parahydrogen

Nuclear spin hyperpolarization (HP) techniques have ushered in a new era of chemically selective magnetic resonance imaging (MRI). By producing MRI signal enhancements that can exceed five orders of magnitude, metabolite fluxes become visible despite their low in vivo concentrations, enabling disease detection and treatment response monitoring without exposure to ionizing radiation.¹ Parahydrogen (pH₂) gas provides a convenient source of singlet nuclear spin order that can be rapidly transformed into MRI-observable proton hyperpolarization through symmetry-breaking hydrogenation chemistry.²⁻⁵ For metabolites like pyruvic acid, the requisite unsaturation for pH₂ addition can be incorporated by esterification to a vinyl or propargyl “side-arm”, as shown in scheme 1 (FIG. 1A).^{6,7} The key step after hydrogenation is the spin order transfer (SOT) to the carbonyl ¹³C to render, after hydrolytic cleavage, a hyperpolarized molecule that was not directly producible by pairwise hydrogenation.

Here we introduce a versatile and efficient process for producing HP ¹³C pyruvate and other carboxylic acids that is amenable to side arm hydrogenation (SAH) of either vinyl or propargyl esters. For allyl ester adducts, this process leverages level anti-crossing (LAC) mediated spin exchange with the initially unpolarized methylene proton(s), as described in our recent publication.⁸

Two approaches to pH₂ SOT have been described in the SAH literature: (1) magnetic field cycling (MFC) through heteronuclear level anti-crossings (LACS) after hydrogenation at μT static magnetic fields, and (2) coherence transfer RF pulse sequences operating on the Î_{z1}Î_{z2} bilinear spin order generated by the PASADENA effect, i.e., hydrogenation at high magnetic field in the weak coupling regime. A practical advantage of MFC-based SOT is that a dedicated NMR spectrometer is not required. Coherence transfer pulse sequences operating on PASADENA spin order have been devised for ethyl and allyl side-arms. Although propargylic side-arms offer greater chemical stability, ease of synthesis, and higher rates of hydrogenation than vinyl esters,^{9,10} the pH₂ sourced protons in the allyl adduct are further away (4 or 5 bonds) from the ¹³C target, and the long-range couplings are too weak to mediate efficient transfer in a single step.¹¹ This motivated invention of two-step relayed coherence transfer pulse sequences: ESOTHERIC and PH-RELAY.^{9,12-14} ESOTHERIC,¹⁴ which is mediated by ¹³C-¹³C coupling and thus requires two-site ¹³C enrichment, has achieved pre-cleavage ¹³C polarizations of up to 8.9% in a cinnamyl pyruvate ester.¹² PH-RELAY, where coherence transfer is relayed via the methylene proton, produced a modest ¹³C polarization of 7% in the 3-d₃-1-d-buten-2-yl [1-¹³C]pyruvate ester despite a theoretical efficiency close to 100%. MINERVA, a recently introduced pulse sequence designed for vinyl ester hydrogenation under PASADENA conditions, has produced batches of per-deuterated ethyl pyruvate with the highest observed ¹³C polarizations to date (c.a. 60%).¹⁵ When combined with a protocol for rapid hydrolysis and evaporative removal of the acetone hydrogenation solvent, ¹³C polarizations of up to 20% in a biocompatible buffer have been realized.¹⁶ The in-vivo images produced by this protocol convincingly demonstrate that pH₂-based HP is a viable alternative to DNP. Building on these outstanding results, we now introduce a hybrid SOT process that offers practical simplifications, improved versatility, and compatibility with flow chemistry techniques.

In the ALTADENA effect, hydrogenation at a low magnetic field (e.g., 5 mT) where all protons are strongly coupled is followed by adiabatic transport to high field for detection in the weak coupling regime. For a two proton adduct such as the d₃-ethyl ester (EPd₃ in FIG. 1.2)), ALTADENA yields a density operator with the form

$${\hat{ρ}}_{f} = \frac{\hat{1}}{4} - {\hat{I}}_{z 1} {\hat{I}}_{z 2} \pm \frac{1}{2} ( {\hat{I}}_{z 1} - {\hat{I}}_{z 2} ) \qquad ( 1 )$$

Note that Equation (1) contains both linear and bilinear spin operator terms. Roughly the same form is obtained in the three-proton allyl ester adducts (APd₂) incorporating pH₂ as H¹ and H² after adiabatic passage through a LAC (referred to as LACADENA), with H¹ replaced by H³:

$${\hat{ρ}}_{f} \approx \frac{\hat{1}}{8} - \frac{1}{2} {\hat{I}}_{z 2} {\hat{I}}_{z 3} \pm \frac{1}{4} ( {\hat{I}}_{z 2} - {\hat{I}}_{z 3} ) \qquad ( 2 )$$

FIG. 2) summarizes the results of our numerical density matrix calculations of the proton spin polarizations, Pᵢ=2Tr(Î_{zi}·ρ_{f}), obtained by adiabatic transport after hydrogenation (with pH₂) of the following per-deuterated vinyl and propargyl esters with various deuterations.

The high polarization of H³ in APd₂ derives from adiabatic passage through a LAC.⁸ Furthermore, the numerical calculations show that deuteration improves the integration of pH₂ singlet order, leading to higher polarization.^{10,17,18}

We now show how the occurrence of both linear and bilinear operator terms affords a choice of coherence transfer pathways. MINERVA exploits the bilinear terms (Î_{z1}Î_{z2} in EPd₃, Î_{z2}Î_{z3} in APd₂) while INEPT works on the Zeeman operators (Î_{z1}−Î_{z2} in EPd₃, Î_{z2}−Î_{z3} in APd₂). The four scenarios are illustrated in FIG. 3.

FIG. 4 presents the results of density matrix simulations of P(¹³C) for the MINERVA and INEPT pulse sequences applied to EPd₃ after PASADENA or ALTADENA preparation. MINERVA achieves P(¹³C)>95% for both initial conditions. Selective INEPT (sINEPT) after ALTADENA achieved similarly high performance, while standard INEPT yielded P(¹³C)≈90%. The analogous simulations for APd₂ presented in FIG. 4 indicate generally lower overall theoretical efficiency, attributed to non-idealities in the LACADENA transformation. Nevertheless, P(¹³C)=−90% is seen in the LACADENA-sINEPT simulation. Lower theoretical polarizations of P(¹³C)=+50% and +70% were obtained with the MINERVA and non-selective INEPT sequences, respectively. Selective proton excitation is also beneficial for carboxylic acids with R groups bearing protons with appreciable coupling to the carbonyl ¹³C (e.g., acetate).

In APd, where only H⁵ is deuterated, adiabatic passage through the LAC divides the terminal proton polarization between the pair of H³ protons in the allyl CH₂ group. This reduces the efficiency of INEPT by roughly a factor of two,^{10,17,18} as confirmed by the numerical simulations presented in FIG. 4. For APd, the INEPT simulation yields P(¹³C)=+32%, while sINEPT yields P(¹³C)−42%. The non-deuterated AP affords significantly lower polarization due to losses stemming from the substantial contribution of the H⁵ proton to the strong coupling eigenstate.

The LACADENA-sINEPT protocol was experimentally demonstrated with the pH₂ addition to [3-d]propargyl [1-¹³C]pyruvate using the apparatus diagramed in FIG. 5. A dual syringe pump (Chemyx 4000) was employed to synchronize infusion of the liquid precursor into the reaction chamber, and withdrawal of liquid hydrogenation adducts through the Varian 400 MHz flow NMR probe. The tubing connecting the withdrawing syringe to the reactor outlet, including the fluid path through the flow probe, was initially filled with acetone-d₆, and the reaction chamber was pressurized to 6 bars with 98% pH₂. The 20 mL infusing syringe was filled with 10 mM Rh(cod)(dppb)BF₄ and 40 mM propargyl ¹³C-pyruvate in acetone-d₆. The precursor solution was infused through a section of copper tubing wrapped around a brass block fitted with a thermocouple and a cartridge-type heating element to heat the precursor solution as it passed through the ultrasonic nozzle into the reaction chamber. The ultrasonic nozzle, vibrating at 120 kHz, produced a fine spray of precursor droplets with a mean diameter of about 13 μm. As the liquid containing hydrogenation products was collected at the bottom of the reaction chamber, the withdrawing syringe was activated to draw the solution into the NMR detection coil. The withdrawing syringe flow rate was set to 3 mL/min to balance losses due to spin relaxation against non-adiabaticity losses during the flow from the reactor to the NMR flow cell.

Each of the hyperpolarized ¹³C spectra of [1-²H]allyl [1-¹³C]pyruvate (APd) presented in FIG. 6 were acquired after infusions of 1.0 mL of precursor solution through the ultrasonic nozzle. Hydrogenations were carried out at either 20° C. or 70° C. The corresponding thermally polarized ¹³C spectra were acquired by averaging 2048 signal transients using a recycle delay sufficient to afford complete spin lattice relaxation. Hydrogenations at ambient temperature produced a maximum signal enhancement of 7,800, corresponding to a ¹³C polarization of 10.3%. At a reaction temperature of 70° C., where the hydrogenation is more rapid, a maximum ¹³C hyperpolarization of P(¹³C)=18.5% was observed, which is 44% of the theoretical maximum.

In summary, we have introduced a hybrid SOT process combining adiabatic longitudinal transport and coherence transfer. Carbonyl ¹³C polarizations of up to 12% were experimentally demonstrated by ultrasonic spray injection of the singly deuterated [3-d]propargyl [1-¹³C]pyruvate. Notably, the deuteration pattern in the doubly deuterated propargyl ester would provide at least a factor of two higher efficiency, as confirmed by numerical density matrix simulations. An advantage of LACADENA-INEPT over relayed coherence transfer sequences for propargyl side-arms is that coherence transfer is achieved in a single step, thereby reducing T₂ decoherence losses. Per-deuterated propargyl and vinyl ester precursors can be expected to exhibit even higher polarization due to the exceptionally long ¹H spin relaxation time. For ethyl esters, substantial coupling between a pH₂-source proton and the carbonyl ¹³C affords the highest attainable polarization. On the other hand, the propargyl ester is a more stable and easily synthesized precursor that exhibits higher reaction rates. Our SOT process is versatile, being applicable to both vinyl and propargyl side-arms without modification.

Coherence transfer after adiabatic longitudinal transport offers practical advantages over PASADENA-based batch reaction configuration. Firstly, hydrogenation is performed outside of the NMR magnet, where the design and construction of a high-performance hydrogenation reactor, such as our ultrasonic spray injection reactor, is not constrained by the limited space within the bore of the NMR magnet. Secondly, the standard INEPT pulse sequence affords a straightforward setup and optimization using Zeeman spin polarization at thermal equilibrium and is already available in the pulse sequence library provided on commercial spectrometers.

The numerical simulations indicate that ¹³C polarizations of up to 90% are possible using LACADENA-sINEPT with propargyl side-arms. For the vinyl esters, the superposition of bilinear and Zeeman order in the ALTADENA density operator affords the choice of coherence transfer method: MINERVA or INEPT. In either case, the theoretical polarization levels are comparable to those of PASADENA-MINERVA. Polarization levels more than 50% attainable by parahydrogen-based methods compete favorably with those of dissolution DNP,^{20,21} but at substantially lower cost, higher reproducibility, shorter polarization times, and perhaps most impactfully, compatibility with flow chemistry and separation techniques.

### REFERENCES FOR EXAMPLE 2

(1) Sun, P.; Wu, Z.; Lin, L.; Hu, G.; Zhang, X.; Wang, J. MR-Nucleomics: The Study of Pathological Cellul(2) Bowers, C. R.; Weitekamp, D. P. Parahydrogen and Synthesis Allow Dramatically Enhanced Nuclear Alignment. J Am Chem Soc 1987, 109 (18), 5541-5542. https://doi.org/10.1021/ja00252a049.
(3) Bowers, C. R.; Weitekamp, D. P. Transformation of Symmetrization Order to Nuclear-Spin Magnetization by Chemical Reaction and Nuclear Magnetic Resonance. Phys Rev Lett 1986, 57 (21), 2645-2648. https://doi.org/10.1103/PhysRevLett.57.2645.
(4) Hövener, J.-B.; Pravdivtsev, A. N.; Kidd, B.; Bowers, C. R.; Glöggler, S.; Kovtunov, K. v; Plaumann, M.; Katz-Brull, R.; Buckenmaier, K.; Jerschow, A.; Reineri, F.; Theis, T.; Shchepin, R. v; Wagner, S.; Bhattacharya, P.; Zacharias, N. M.; Chekmenev, E. Y. Parahydrogen-Based Hyperpolarization for Biomedicine. Angewandte Chemie International Edition 2018, 57 (35), 11140-11162. https://doi.org/10.1002/anie.201711842.
(5) Schmidt, A. B.; Bowers, C. R.; Buckenmaier, K.; Chekmenev, E. Y.; de Maissin, H.; Eills, J.; Ellermann, F.; Glöggler, S.; Gordon, J. W.; Knecht, S.; Koptyug, I. v; Kuhn, J.; Pravdivtsev, A. N.; Reineri, F.; Theis, T.; Them, K.; Hövener, J.-B. Instrumentation for Hydrogenative Parahydrogen-Based Hyperpolarization Techniques. Anal Chem 2022, 94 (1), 479-502. https://doi.org/10.1021/acs.analchem.1c04863.
(6) Reineri, F.; Boi, T.; Aime, S. ParaHydrogen Induced Polarization of 13C Carboxylate Resonance in Acetate and Pyruvate. Nat Commun 2015, 6 (1), 5858. https://doi.org/10.1038/ncomms6858.
(7) Cavallari, E.; Carrera, C.; Sorge, M.; Bonne, G.; Muchir, A.; Aime, S.; Reineri, F. The 13C Hyperpolarized Pyruvate Generated by ParaHydrogen Detects the Response of the Heart to Altered Metabolism in Real Time. Sci Rep 2018, 8 (1), 8366. https://doi.org/10.1038/s41598-018-26583-2.
(8) Ferrer, M.-J.; Kuker, E. L.; Semenova, E.; Gangano, A. J.; Lapak, M. P.; Grenning, A. J.; Dong, V. M.; Bowers, C. R. Adiabatic Passage through Level Anticrossings in Systems of Chemically Inequivalent Protons Incorporating Parahydrogen: Theory, Experiment, and Prospective Applications. J Am Chem Soc 2022, 144 (45), 20847-20853. ar Processes with Multinuclear Magnetic Resonance Spectroscopy and Imaging in Vivo. NMR Biomed 2022, e4845
(1) Sun, P.; Wu, Z.; Lin, L.; Hu, G.; Zhang, X.; Wang, J. MR-Nucleomics: The Study of Pathological Cellular Processes with Multinuclear Magnetic Resonance Spectroscopy and Imaging in Vivo. NMR Biomed 2022, e4845. https://doi.org/10.1002/NBM.4845.
(2) Bowers, C. R.; Weitekamp, D. P. Parahydrogen and Synthesis Allow Dramatically Enhanced Nuclear Alignment. J Am Chem Soc 1987, 109 (18), 5541-5542. https://doi.org/10.1021/ja00252a049.
(3) Bowers, C. R.; Weitekamp, D. P. Transformation of Symmetrization Order to Nuclear-Spin Magnetization by Chemical Reaction and Nuclear Magnetic Resonance. Phys Rev Lett 1986, 57 (21), 2645-2648. https://doi.org/10.1103/PhysRevLett.57.2645.
(4) Hövener, J.-B.; Pravdivtsev, A. N.; Kidd, B.; Bowers, C. R.; Glöggler, S.; Kovtunov, K. v; Plaumann, M.; Katz-Brull, R.; Buckenmaier, K.; Jerschow, A.; Reineri, F.; Theis, T.; Shchepin, R. v; Wagner, S.; Bhattacharya, P.; Zacharias, N. M.; Chekmenev, E. Y. Parahydrogen-Based Hyperpolarization for Biomedicine. Angewandte Chemie International Edition 2018, 57 (35), 11140-11162. https://doi.org/10.1002/anie.201711842.
(5) Schmidt, A. B.; Bowers, C. R.; Buckenmaier, K.; Chekmenev, E. Y.; de Maissin, H.; Eills, J.; Ellermann, F.; Glöggler, S.; Gordon, J. W.; Knecht, S.; Koptyug, I. v; Kuhn, J.; Pravdivtsev, A. N.; Reineri, F.; Theis, T.; Them, K.; Hövener, J.-B. Instrumentation for Hydrogenative Parahydrogen-Based Hyperpolarization Techniques. Anal Chem 2022, 94 (1), 479-502. https://doi.org/10.1021/acs.analchem.1c04863.
(6) Reineri, F.; Boi, T.; Aime, S. ParaHydrogen Induced Polarization of 13C Carboxylate Resonance in Acetate and Pyruvate. Nat Commun 2015, 6 (1), 5858. https://doi.org/10.1038/ncomms6858.
(7) Cavallari, E.; Carrera, C.; Sorge, M.; Bonne, G.; Muchir, A.; Aime, S.; Reineri, F. The 13C Hyperpolarized Pyruvate Generated by ParaHydrogen Detects the Response of the Heart to Altered Metabolism in Real Time. Sci Rep 2018, 8 (1), 8366. https://doi.org/10.1038/s41598-018-26583-2.
(8) Ferrer, M.-J.; Kuker, E. L.; Semenova, E.; Gangano, A. J.; Lapak, M. P.; Grenning, A. J.; Dong, V. M.; Bowers, C. R. Adiabatic Passage through Level Anticrossings in Systems of Chemically Inequivalent Protons Incorporating Parahydrogen: Theory, Experiment, and Prospective Applications. J Am Chem Soc 2022, 144 (45), 20847-20853. https://doi.org/10.1021/jacs.2c09000.
(9) Svyatova, A.; Kozinenko, V. P.; Chukanov, N. v; Burueva, D. B.; Chekmenev, E. Y.; Chen, Y.-W.; Hwang, D. W.; Kovtunov, K. v; Koptyug, I. v. PHIP Hyperpolarized [1-13C]Pyruvate and [1-13C]Acetate Esters via PH-INEPT Polarization Transfer Monitored by 13C NMR and MRI. Sci Rep 2021, 11 (1), 5646. https://doi.org/10.1038/s41598-021-85136-2.
(10) Marshall, A.; Salhov, A.; Gierse, M.; Müller, C.; Keim, M.; Lucas, S.; Parker, A.; Scheuer, J.; Vassiliou, C.; Neumann, P.; Jelezko, F.; Retzker, A.; Blanchard, J. W.; Schwartz, I.; Knecht, S. Radio-Frequency Sweeps at {\mu}T Fields for Parahydrogen-Induced Polarization of Biomolecules. ArXiv 2022, May 31. https://doi.org/10.48550/arxiv.2205.15709.
(11) Stewart, N. J.; Kumeta, H.; Tomohiro, M.; Hashimoto, T.; Hatae, N.; Matsumoto, S. Long-Range Heteronuclear J-Coupling Constants in Esters: Implications for 13C Metabolic MRI by Side-Arm Parahydrogen-Induced Polarization. Journal of Magnetic Resonance 2018, 296, 85-92. https://doi.org/10.1016/J.JMR.2018.08.009.
(12) Korchak, S.; Yang, S.; Mamone, S.; Glöggler, S. Pulsed Magnetic Resonance to Signal-Enhance Metabolites within Seconds by Utilizing Para-Hydrogen. ChemistryOpen 2018, 7 (5), 344-348. https://doi.org/10.1002/open.201800024.
(13) Dagys, L.; Jagtap, A. P.; Korchak, S.; Mamone, S.; Saul, P.; Levitt, M. H.; Glöggler, S. Nuclear Hyperpolarization of (1-13C)-Pyruvate in Aqueous Solution by Proton-Relayed Side-Arm Hydrogenation. Analyst 2021, 146 (5), 1772-1778. https://doi.org/10.1039/D0AN02389B.
(14) Ding, Y.; Korchak, S.; Mamone, S.; Jagtap, A. P.; Stevanato, G.; Sternkopf, S.; Moll, D.; Schroeder, H.; Becker, S.; Fischer, A.; Gerhardt, E.; Outeiro, T. F.; Opazo, F.; Griesinger, C.; Glöggler, S. Rapidly Signal-Enhanced Metabolites for Atomic Scale Monitoring of Living Cells with Magnetic Resonance. Chemistry—Methods 2022, e202200023. https://doi.org/10.1002/CMTD.202200023.
(15) Korchak, S.; Mamone, S.; Glöggler, S. Over 50% 1H and 13C Polarization for Generating Hyperpolarized Metabolites-A Para-Hydrogen Approach. ChemistryOpen 2018, 7 (9), 672-676. https://doi.org/10.1002/open.201800086.
(16) Hune, T.; Mamone, S.; Schroeder, H.; Jagtap, A. P.; Sternkopf, S.; Stevanato, G.; Korchak, S.; Fokken, C.; Müller, C. A.; Schmidt, A. B.; Becker, D.; Glöggler, S. Metabolic Tumor Imaging with Rapidly Signal?Enhanced 1?13 C?Pyruvate?d 3. ChemPhysChem 2022. https://doi.org/10.1002/cphc.202200615.
(17) Levitt, M. H. Symmetry Constraints on Spin Dynamics: Application to Hyperpolarized NMR. Journal of Magnetic Resonance 2016, 262, 91-99. https://doi.org/10.1016/J.JMR.2015.08.021.
(18) Nielsen, N. C.; Schulte-Herbrüggen, T.; Sørensen, O. W. Bounds on Spin Dynamics Tightened by Permutation Symmetry Application to Coherence Transfer in 12S and 13S Spin Systems. Mol Phys 1995, 85 (6), 1205-1216. https://doi.org/10.1080/00268979500101771.
(19) Hogben, H. J.; Krzystyniak, M.; Charnock, G. T. P.; Hore, P. J.; Kuprov, I. Spinach A Software Library for Simulation of Spin Dynamics in Large Spin Systems. Journal of Magnetic Resonance 2011, 208 (2), 179-194. https://doi.org/10.1016/J.JMR.2010.11.008.
(20) Ardenkjaer-Larsen, J. H. On the Present and Future of Dissolution-DNP. Journal of Magnetic Resonance 2016, 264, 3-12. https://doi.org/10.1016/j.jmr.2016.01.015.
(21) Ardenkjaer-Larsen, J. H.; Fridlund, B.; Gram, A.; Hansson, G.; Hansson, L.; Lerche, M. H.; Servin, R.; Thaning, M.; Golman, K. Increase in Signal-to-Noise Ratio of >10,000 Times in Liquid-State NMR. Proc Natl Acad Sci USA 2003, 100 (18), 10158-10163.

### Example 3: Combining Adiabatic Passage and Coherence Transfer for Efficient Spin Order Transfer in Allyl Ester Adducts of Parahydrogen

Adiabatic passage through a ¹H-¹H level anti-crossing (LAC) is combined with selective INEPT coherence transfer to transform parahydrogen sourced spin order into carbonyl ¹³C magnetization in a deuterated allyl pyruvate adduct, resulting in ¹³C polarization of up to 11±1%. For allylic esters, this approach affords scalability of hyperpolarized metabolites and high polarization.

Parahydrogen (pH₂), the singlet spin isomer of dihydrogen, is an inexpensive source of nuclear spin order that can be rapidly transformed into NMR-observable spin order by reversible or irreversible hydrogenation chemistry.¹⁻³ With NMR signal enhancements that can easily exceed 104, parahydrogen-based hyperpolarization has shown excellent potential for clinical magnetic resonance imaging.^{4,5} A limitation of the technique, however, is that hydrogenation is applicable only to molecules and complexes that possess the appropriate chemical unsaturation. In the Side-Arm Hydrogenation (SAH) technique, a carboxylic acid metabolite of interest (e.g., pyruvic acid) is esterified with a vinyl or propargyl group to provide a means of incorporating pH₂. After hydrogenation, the pH₂-sourced proton spin order needs to be transferred across the ester linkage to the carbonyl ¹³C, followed by cleavage of the spent side-arm by hydrolysis.

Here we present a novel spin order transfer (SOT) process for allyl ester hydrogenation adducts that combines adiabatic passage through a ¹H-¹H level anti-crossing (LAC)⁶ and coherence transfer by the re-focused INEPT pulse sequence.⁷ In this hybrid SOT protocol, which we refer to as LACADENA-INEPT (level anti-crossing after dissociation engenders net alignment —insensitive nuclei enhancement by polarization transfer), the spin order of the pH₂-sourced protons during ALTADENA type experiment is transferred to the methylene proton(s) in allyl ester hydrogenation adducts by adiabatic passage through a LAC induced by proton-proton spin coupling.⁶ The Zeeman order so induced sets up an initial condition that is amenable to single-step INEPT coherence transfer to the carbonyl ¹³C while also not being confined the limited space of a superconducting magnet during hydrogenation.

FIG. 4 summarizes the results of numerical density matrix calculations⁸ of the proton spin polarizations, Pᵢ=2Tr(Î_{iz}·ρ_{f}), where ·\hat{ρ}_{f} is the density matrix obtained by simulation of adiabatic transport after hydrogenation (with 100% pH₂) of a selectively deuterated propargyl ester and Î_{iz} is the z-component of the spin angular momentum operator of the i^{th} proton.

For propargylic side-arms, we show that LACADENA-selective INEPT (sINEPT) can deliver higher pyruvate ¹³C polarization levels than other SOT schemes while also offering key practical advantages.

The original demonstration of SOT after SAH was performed by field cycling at ultra-low magnetic fields. Carbonyl ¹³C polarization values in the 1.5-5% range were obtained after hydrogenation of propargyl or phenyl acetylene esters.⁹⁻¹⁴ Deuteration can help to preserve the integrity of the singlet spin order through hydrogenation,⁶ but due to rapid quadrupolar relaxation, the gain is offset by significant losses in the regime of strong heteronuclear coupling.¹⁵ In Ref.¹⁶ it was shown that this relaxation mechanism can be circumvented by radio-frequency field sweeps at μT fields, thereby increasing the polarization to 9.8% in selectively deuterated cinnamyl pyruvate. Pulse sequences have been devised for SOT to the carbonyl ¹³C after SAH of vinylic or propargylic esters with pH₂ at high magnetic field. These coherence transfer sequences operate on the Î_{1z}Î_{2z} bilinear spin order generated by the PASADENA effect,^{1,2} i.e., hydrogenation at high magnetic field in the weak coupling regime.

While propargylic side-arms offer greater chemical stability, ease of synthesis, and higher rates of hydrogenation than vinyl esters,¹⁷ the pH₂ sourced protons in allyl adducts are further away (4-5 bonds) from the carbonyl ¹³C target, and the long-range couplings are too weak to mediate efficient transfer in a single step.¹⁸ This motivated invention of two-step relayed coherence transfer pulse sequences^{17,19,20} such as ESOTHERIC,¹⁹ where coherence transfer is mediated by ¹³C-13C coupling in doubly ¹³C labelled precursors. Pre-cleavage ¹³C polarizations of up to 8.9% were demonstrated in a cinnamyl pyruvate ester.¹⁹ In PH-RELAY, coherence transfer is mediated by the methylene proton in two steps. Although the theoretical efficiency approaches 100%, a ¹³C polarization of 7% in the 3-d₃-1-d-buten-2-yl [1-13C]pyruvate ester was reported.²⁰

The timing diagram for LACADENA-INEPT is shown in FIG. 3. Hydrogenation to the allyl ester is performed at a magnetic field (e.g., the fringe field of a superconducting NMR magnet) that is low enough to induce strong coupling among protons yet high enough to preserve weak coupling to heteronuclei (e.g., ¹³C and ²H)—i.e., where the difference in Larmor frequencies is significantly greater than the heteronuclear coupling. As shown previously,⁶ the singlet is an approximate eigenstate in the allyl adducts, especially for (Z)-allyl-3-d 2-oxopropanoate-1-¹³C (APd) and (Z)-allyl-1,3-d 2-oxopropanoate-1-¹³C (APd₂). Adiabatic passage through a LAC induced by the proton-proton scalar coupling was previously shown to elicit efficient exchange of ALTADENA spin order with the methylene proton(s). The net alignment Zeeman order after this LACADENA transformation is amenable to single-step INEPT to the carbonyl ¹³C.

Numerical density matrix simulations of the maximum ¹³C polarization, P(¹³C), resulting from application of the INEPT or sINEPT pulse sequences to AP, APd, and APd₂ after LACADENA preparation are summarized in FIG. 4. For APd₂, P(¹³C)=−90% by LACADENA-sINEPT, while +70% is theoretically attainable by non-selective INEPT, respectively. The benefit of selective proton excitation should also extend to carboxylic acids with R groups bearing protons with appreciable coupling to the carbonyl ¹³C, as in allyl acetate. In APd, where only H⁵ is deuterated, adiabatic passage through the LAC divides the terminal proton polarization between the pair of H³ protons in the allyl CH₂ group. This reduces the efficiency of INEPT by roughly a factor of two,^{16,21,22} as confirmed by the numerical simulations presented in FIG. 4. For APd, the INEPT simulation indicates P(¹³C)=+32%, while sINEPT yields P(¹³C) −42%. The non-deuterated AP shows significantly lower polarization stemming from the losses incurred by the large contribution of the H⁵ proton to the low-field eigenstate.

We demonstrate the LACADENA-sINEPT protocol using a modified version of the apparatus described in Ref.⁶ Briefly, a dual syringe pump (Chemyx 4000) is employed to synchronize infusion of the reactant solution into the reaction chamber with the withdrawal of liquid hydrogenation products into a Varian 400 MHz flow NMR probe. The tubing connecting the withdrawing syringe to the reactor outlet, including the fluid path incorporating the NMR flow cell, is initially filled with neat acetone-d₆, and the reaction chamber is pressurized to 6 bars with 98% para-enriched H₂. The 20 mL infusing syringe is filled with a precursor solution containing 10 mM Rh(cod)(dppb)BF₄ and 40 mM AP or APd substrate in acetone-d₆. Details of the syntheses of AP and APd are provided in Ref.⁶ The precursor solution is infused at 5 mL/min through a section of 1/16 O.D. inch copper tubing wound around a brass block fitted with a thermocouple and a cartridge-type heating element to heat the liquid to the desired reaction temperature before entering the ultrasonic nozzle into the reaction chamber. The hydrogenation reaction occurred in the fringe field of superconducting magnet, approximately 2.5 mT. The ultrasonic nozzle, excited at 120 kHz, produces a fine spray of precursor droplets with a mean diameter of about 13 μm.²³ The liquid hydrogenation adducts accumulate at the bottom of the funnel-shaped reaction chamber. After 5 s of infusion, the withdrawing syringe is activated to draw the solution into the magnetic field and NMR flow cell. The withdrawing syringe flow rate of 3 mL/min is employed to balance polarization losses due to spin relaxation against non-adiabaticity during transport from the reactor to the NMR flow cell.

FIG. 6 presents the LACADENA-sINEPT ¹³C NMR spectra acquired at 9.4 T after hydrogenation of APd at either 20 or 70° C. The corresponding thermally polarized ¹³C spectra were acquired after waiting 10 min to allow complete relaxation of the hyperpolarization. The ¹³C signal enhancements and spin polarizations were calculated from the signal integrals which include the Ernst condition correction.

For hydrogenation at 70° C., a maximum P(¹³C) hyperpolarization of between 10.4 and 11.9% is estimated. The range represents the systematic error due to the uncertainty in the 2^{nd} hydrogenation to propyl ester. The lower limit is obtained assuming that the 2^{nd} reduction occurred inside the NMR flow cell after acquisition of the HP ¹³C spectrum, while the upper value of 11.9% assumes that hydrogenation was complete prior to acquisition of the HP ¹³C spectrum. The observed P(¹³C) levels are only about 28% of the theoretical maximum. The losses are attributed mainly to spin-lattice relaxation during accumulation of adducts and transport to high field. To our knowledge, low-field Tₛ (the lifetime of the singlet-triplet imbalance) has not been measured for APd, but it is likely to be significantly longer than the high field spin-lattice relaxation time of the protons at 9.4 T (c.a., τ₁=6.4 s).

### Conclusions

We have demonstrated a new hybrid spin order transfer process combining (1) ultrasonic spray injection hydrogenation of a selectively deuterated propargyl pyruvate precursor, (2) adiabatic longitudinal transport through a level anti-crossing, and (3) selective INEPT coherence transfer across the ester linkage. Pyruvate carbonyl ¹³C polarizations of up to 11±1% were recorded, which is higher than what has been reported for two-step coherence transfer pulse sequences or magnetic field cycling schemes. Notably, the deuteration pattern in the doubly deuterated propargyl ester (e.g., APd₂) is expected to automatically increase the efficiency by a factor of two, according to symmetry considerations and numerical density matrix simulations. It could be more than a factor of two because the spin-lattice relaxation time of the single CHD proton in APd₂ is likely longer than that of the CH₂ proton pair in APd. An advantage of the LACADENA-sINEPT approach over relayed coherence transfer sequences for propargyl SAH is that coherence transfer is achieved in a single step, thereby reducing τ₂ decoherence losses. On the other hand, high efficiency requires a short transport time that maintains adiabaticity. Per-deuterated vinyl ester precursors can be expected to exhibit higher polarizations due to the exceptionally long ¹H spin relaxation time. For ethyl esters, substantial coupling between a pH₂-source proton and the carbonyl ¹³C affords the highest attainable polarization in the absence of LAC-induced SOT. On the other hand, the propargyl ester is a more stable and easily synthesized precursor with higher reaction rates. The ALTADENA/LACADENA-INEPT process is versatile, being applicable to both vinyl and propargyl side-arms.

From a technical aspect, the LACADENA-INEPT experimental arrangement offers practical advantages over the standard PASADENA-based batch reaction processes utilizing in-situ bubbling of pH₂ through the NMR sample tube. First, hydrogenation is performed outside of the NMR magnet, where the design and construction of a high-performance hydrogenation reactor, such as our ultrasonic spray injection reactor, is not constrained by the limited space within the bore of the NMR magnet. Secondly, the standard INEPT pulse sequence affords a straightforward setup and optimization using ordinary Zeeman spin polarization at thermal equilibrium. Moreover, INEPT pulse sequences are provided in the standard pulse sequence library on commercial spectrometers. Non-selective INEPT can be employed, albeit with reduced efficiency, when selective excitation is not supported by the hardware or at lower magnetic fields where frequency selective excitation is infeasible.

Numerical simulations indicate that ¹³C polarizations of up to 90% are possible using LACADENA-sINEPT in the doubly deuterated APd₂ ester. At the present time, only APd was available. Nevertheless, extrapolation of our experimental results to APd₂ suggests that the attainable polarization levels>20% (pre-cleavage). This compares favorably with dissolution DNP,^{24,25} but with substantially lower cost, higher reproducibility, and shorter polarization times.

### NOTES AND REFERENCES FOR EXAMPLE 3

1 C. R. Bowers and D. P. Weitekamp, J Am Chem Soc, 1987, 109, 5541-5542.
2 C. R. Bowers and D. P. Weitekamp, Phys Rev Lett, 1986, 57, 2645-2648.
3 R. W. Adams, J. A. Aguilar, K. D. Atkinson, M. J. Cowley, P. I. P. Elliott, S. B. Duckett, G. G. R. Green, I. G. Khazal, J. Lopez-Serrano and D. C. Williamson, Science (1979), 2009, 323, 1708-1711.
4 J. B. Hovener, A. N. Pravdivtsev, B. Kidd, C. R. Bowers, S. Gloggler, K. v. Kovtunov, M. Plaumann, R. Katz-Brull, K. Buckenmaier, A. Jerschow, F. Reineri, T. Theis, R. v. Shchepin, S.
A. B. Schmidt, C. R. Bowers, K. Buckenmaier, E. Y. Chekmenev, H. de Maissin, J. Eills, F. Ellermann, S. Glöggler, J. W. Gordon, S. Knecht, I. V Koptyug, J. Kuhn, A. N. Pravdivtsev, F. Reineri, T. Theis, K. Them and J.-B. Hövener, Anal Chem, 2022, 94, 479-502.
6 M.-J. Ferrer, E. L. Kuker, E. Semenova, A. J. Gangano, M. P. Lapak, A. J. Grenning, V. M. Dong and C. R. Bowers, J Am Chem Soc, 2022, 144, 20847-20853.
7 G. A. Morris and R. Freeman, J Am Chem Soc, 2002, 101, 760-762.
8 H. J. Hogben, M. Krzystyniak, G. T. P. Charnock, P. J. Hore and I. Kuprov, Journal of Magnetic Resonance, 2011, 208, 179-194.
9 E. Cavallari, C. Carrera, S. Aime and F. Reineri, Chemistry—A European Journal, 2017, 23, 1200-1204.
10 E. Cavallari, C. Carrera, S. Aime and F. Reineri, Journal of Magnetic Resonance, 2018, 289, 12-17.
11 F. Reineri, T. Boi and S. Aime, Nat Commun, 2015, 6, 5858.
12 R. V. Shchepin, D. A. Barskiy, A. M. Coffey, I. v. Manzanera Esteve and E. Y. Chekmenev, Angewandte Chemie International Edition, 2016, 55, 6071-6074.
13 E. Cavallari, C. Carrera, M. Sorge, G. Bonne, A. Muchir, S. Aime and F. Reineri, Sci Rep, 2018, 8, 8366.
14 N. V. Chukanov, O. G. Salnikov, R. V. Shchepin, K. V. Kovtunov, I. V. Koptyug and E. Y. Chekmenev, ACS Omega, 2018, 3, 6673-6682.
15 M. C. D. Tayler and L. F. Gladden, Journal of Magnetic Resonance, 2019, 298, 101-106.
16 A. Marshall, A. Salhov, M. Gierse, C. Müller, M. Keim, S. Lucas, A. Parker, J. Scheuer, C. Vassiliou, P. Neumann, F. Jelezko, A. Retzker, J. W. Blanchard, I. Schwartz and S. Knecht, ArXiv, DOI:10.48550/arxiv.2205.15709.
17 A. Svyatova, V. P. Kozinenko, N. V Chukanov, D. B. Burueva, E. Y. Chekmenev, Y.-W. Chen, D. W. Hwang, K. V Kovtunov and I. V Koptyug, Sci Rep, 2021, 11, 5646.
18 N. J. Stewart, H. Kumeta, M. Tomohiro, T. Hashimoto, N. Hatae and S. Matsumoto, Journal of Magnetic Resonance, 2018, 296, 85-92.
19 S. Korchak, S. Yang, S. Mamone and S. Glöggler, ChemistryOpen, 2018, 7, 344-348.
20 L. Dagys, A. P. Jagtap, S. Korchak, S. Mamone, P. Saul, M. H. Levitt and S. Glöggler, Analyst, 2021, 146, 1772-1778.
21 M. H. Levitt, Journal of Magnetic Resonance, 2016, 262, 91-99.
22 N. C. Nielsen, T. Schulte-Herbrüggen and O. W. Sørensen, Mol Phys, 1995, 85, 1205-1216.
23 Sono-Tek product information, https://www.sono-tek.com, (accessed 10 Feb. 2023).
24 J. H. Ardenkjaer-Larsen, Journal of Magnetic Resonance, 2016, 264, 3-12.
25 J. H. Ardenkjaer-Larsen, B. Fridlund, A. Gram, G. Hansson, L. Hansson, M. H. Lerche, R. Servin, M. Thaning and K. Golman, Proc Natl Acad Sci USA, 2003, 100, 10158-10163.

### Example 4

Adiabatic passage through a level anti-crossing (LAC) induced by ¹H-¹H coupling is combined with selective INEPT coherence transfer to transform parahydrogen sourced spin order into carbonyl ¹³C magnetization in a deuterated allyl pyruvate adduct, resulting in ¹³C polarization of up to 11±1%. For allylic esters, this approach affords technical advantages for production of hyperpolarized metabolites.

Parahydrogen (pH₂), the metastable singlet spin isomer of dihydrogen, is an inexpensive source of nuclear spin order that can be rapidly transformed into NMR-observable spin order by reversible or irreversible hydrogenation chemistry.¹⁻³ With NMR signal enhancements that can easily exceed 104, parahydrogen-based hyperpolarization has shown excellent potential for clinical magnetic resonance imaging.⁴⁵ A limitation of the technique, however, is that hydrogenation is applicable only to molecules and complexes that possess the appropriate chemical unsaturation. In the Side-Arm Hydrogenation (SAH) technique, a carboxylic acid metabolite of interest (e.g., pyruvic acid) is esterified with a vinyl or propargyl group to provide a means of incorporating pH₂. After hydrogenation, the pH₂-sourced proton spin order needs to be transferred across the ester linkage to the carbonyl ¹³C, followed by cleavage of the spent side-arm by hydrolysis.

Here we present a novel spin order transfer (SOT) process for allyl ester hydrogenation adducts that combines adiabatic passage through a homonuclear level anti-crossing (LAC)⁶ and coherence transfer by the re-focused INEPT pulse sequence.⁷ In this hybrid SOT protocol, the spin order of the pH₂-sourced protons is translated to Zeeman order of the methylene proton(s) in allyl ester hydrogenation adducts by adiabatic passage through a LAC induced by proton-proton spin coupling.⁶ This variation of the ALTADENA effect,⁸ termed LACADENA, sets up an initial condition that is amenable to single-step INEPT coherence transfer to the carbonyl ¹³C.

Scheme 1 summarizes the results of numerical density matrix calculations⁹ of the proton spin polarizations, Pᵢ=2Tr(Î_{zi}·\hat{ρ}_{f}), where ·\hat{ρ}_{f} is the density matrix obtained by simulation of adiabatic transport after hydrogenation (with 100% pH₂) of a selectively deuterated propargyl ester and Î_{zi} is the z-component of the spin angular momentum operator of the i^{th} proton.

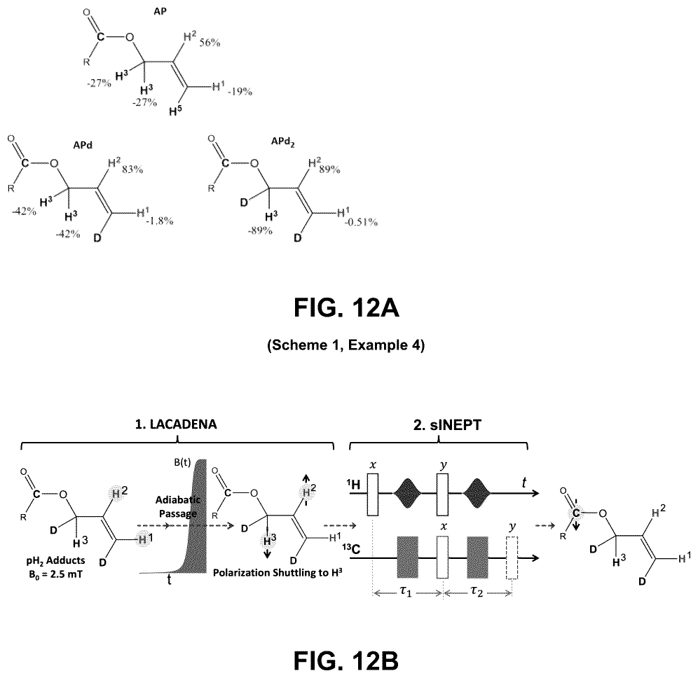

Scheme 1, as shown in FIG. 12A, illustrates a simulated ¹H polarizations in allyl 2-oxopropanoate-1-¹³C (AP); (Z)-allyl-3-d 2-oxopropanoate-1-¹³C (APd); (Z)-allyl-1,3-d 2-oxopropanoate-1-¹³C (APd₂) after adiabatic passage through a LAC. H¹ and H² originate from pH₂. These simulations were performed using the field profile measured along the flow path from the reactor (located below the opening of the magnet) to the field center of the 9.4 T superconducting magnet used in the experiments described below.

For a metabolite with a propargylic side-arm, we show that selective INEPT (sINEPT),^{10,11} when applied after LACADENA preparation, can deliver higher pyruvate ¹³C polarization levels than what has been reported in the literature using other SOT schemes while at the same time offering practical advantages.

The original demonstration of SOT after SAH was performed by field cycling at ultra-low magnetic fields. Carbonyl ¹³C polarization values in the 1.5-5% range were obtained after hydrogenation of propargyl or phenyl acetylene esters.¹²⁻¹⁷ Deuteration can help to preserve the integrity of the singlet spin order through hydrogenation,⁶ but due to rapid quadrupolar relaxation, the gain is offset by significant losses in the regime of strong heteronuclear coupling.¹⁸ In Ref. ¹⁹ it was shown that this relaxation mechanism can be circumvented by radio-frequency field sweeps at μT fields, thereby increasing the polarization to 9.8% in selectively deuterated cinnamyl pyruvate. Pulse sequences have been devised for SOT to the carbonyl ¹³C after SAH of vinylic or propargylic esters with pH₂ at high magnetic field. These coherence transfer sequences operate on the Î_{z1}Î_{z2} bilinear spin order generated by the PASADENA effect,^{1,2} i.e., hydrogenation at high magnetic field in the weak coupling regime.

Propargylic side-arms offer greater chemical stability, ease of synthesis, and higher rates of hydrogenation than vinyl ester.²⁰ Disadvantageously, the pH₂ sourced protons in allyl adducts are further away (4-5 bonds) from the carbonyl ¹³C target, and the long-range couplings are too weak to mediate efficient transfer in a single step.²¹ This motivated invention of two-step relayed coherence transfer pulse sequences^{20,22,23} such as ESOTHERIC,²² where coherence transfer is mediated by ¹³C-¹³C coupling in doubly ¹³C labelled precursors. Pre-cleavage ¹³C polarizations of up to 8.9% were demonstrated in a cinnamyl pyruvate ester.²² In PH-RELAY, coherence transfer is mediated by the methylene proton in two steps. Although the theoretical efficiency approaches 100%, a ¹³C polarization of only 7% in the 3-d₃-1-d-buten-2-yl [1-¹³C]pyruvate ester was realized in experiments.²³

The timing diagram for LACADENA-INEPT is shown in FIG. 12B. Hydrogenation to the allyl ester is performed at a magnetic field (e.g., the fringe field of a superconducting NMR magnet) that is low enough to induce strong coupling among protons yet high enough to preserve weak coupling to heteronuclei (e.g., ¹³C and ²H)—i.e., where the difference in Larmor frequencies is significantly greater than the heteronuclear coupling. As shown previously,⁶ the singlet is an approximate eigenstate in the allyl adducts, especially for APd and APd₂. Adiabatic passage through a LAC induced by the proton-proton scalar coupling was previously shown to elicit efficient exchange of ALTADENA spin order with the methylene proton(s). The net alignment Zeeman order after this LACADENA transformation is suitable for single-step INEPT transfer to the carbonyl ¹³C.

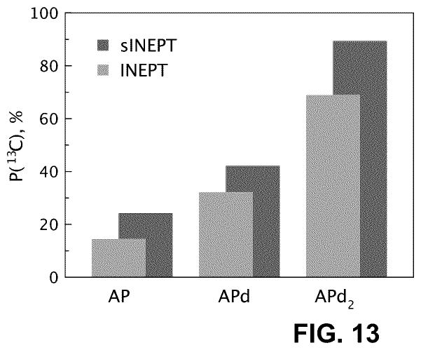

Numerical density matrix simulations of the maximum ¹³C polarization, P(¹³C), resulting from application of the INEPT or sINEPT pulse sequences to AP, APd, and APd₂ after LACADENA preparation are summarized in FIG. 13. For APd₂, P(¹³C)=−90% by LACADENA-sINEPT, while +70% is theoretically attainable by non-selective INEPT, respectively. The benefit of selective proton excitation should also extend to carboxylic acids with R groups bearing protons with appreciable coupling to the carbonyl ¹³C, as in allyl acetate. In APd, where only H⁵ is deuterated, adiabatic passage through the LAC divides the terminal proton polarization between the pair of H³ protons in the allyl CH₂ group. This reduces the efficiency of INEPT by roughly a factor of two,^{19,24,25} as confirmed by the numerical simulations presented in FIG. 13. For APd, the INEPT simulation indicates P(¹³C)=+32%, while sINEPT yields P(¹³C) −42%. The non-deuterated AP shows significantly lower polarization stemming from the losses incurred by the large contribution of the H⁵ proton to the low-field eigenstate.

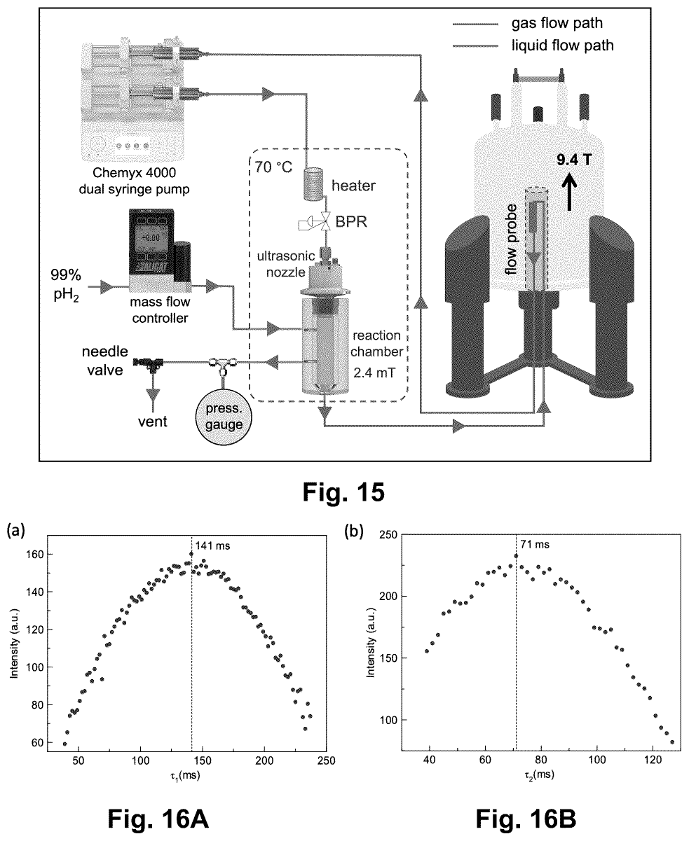

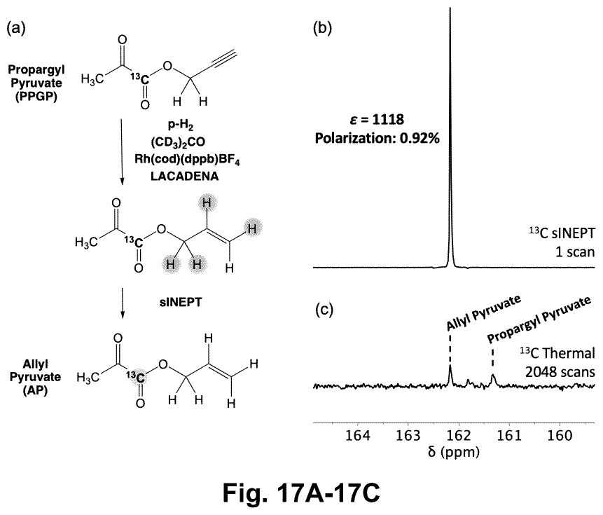

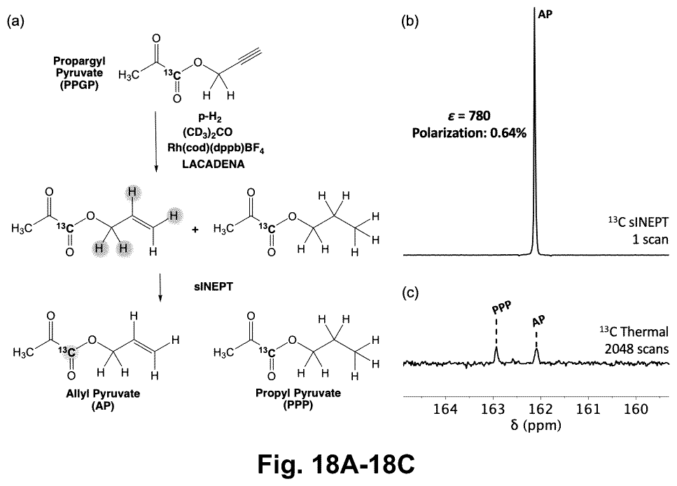

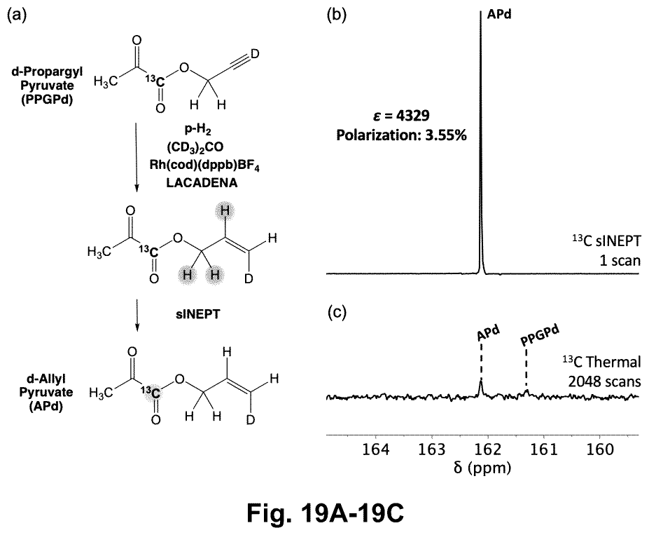

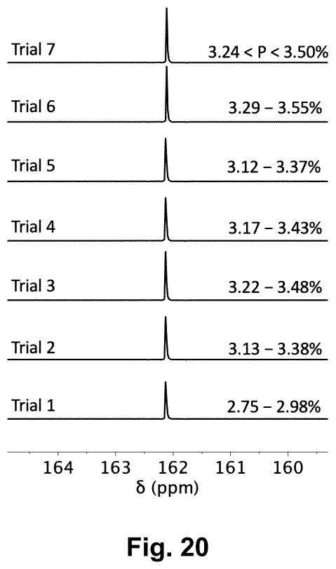

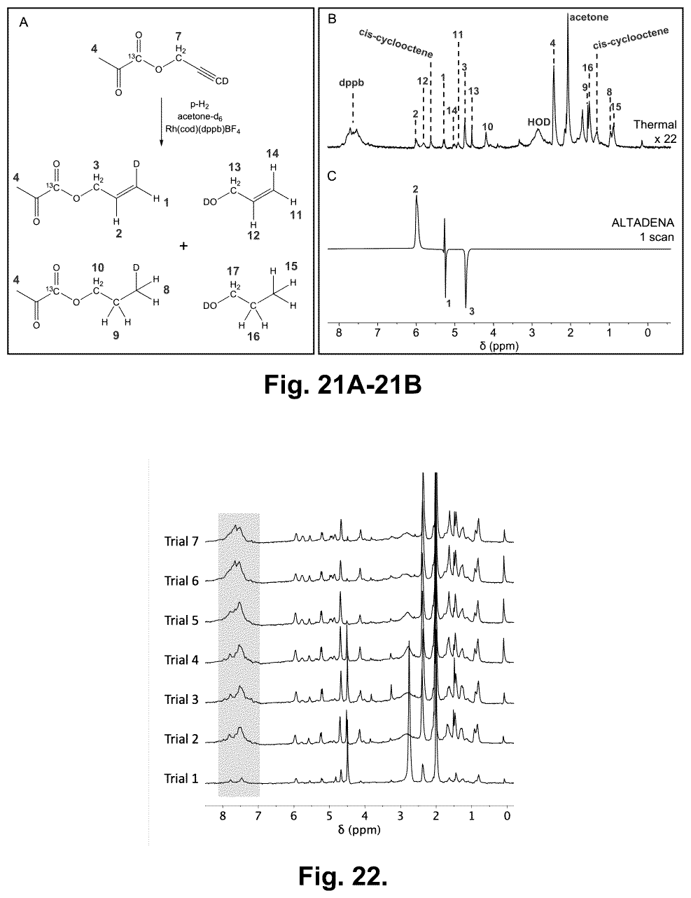

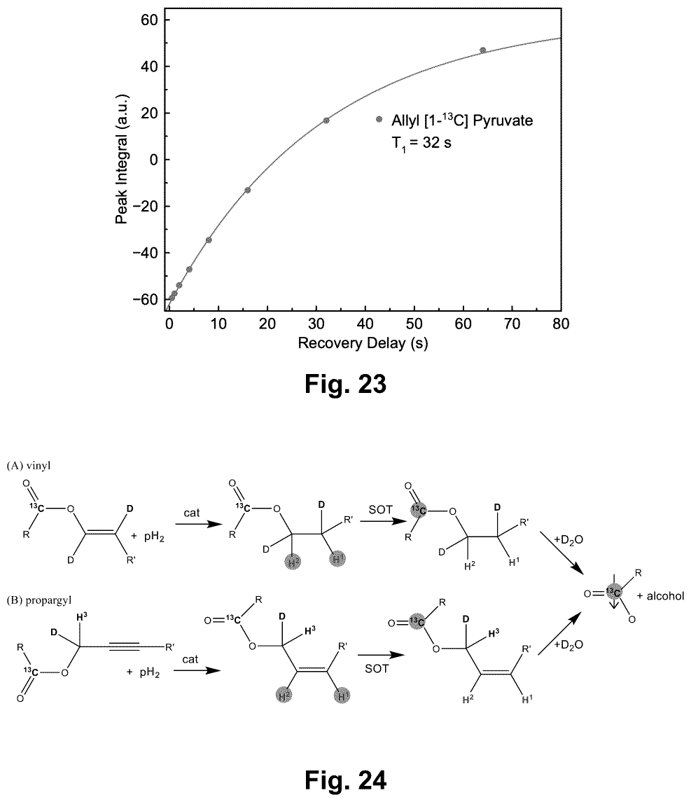

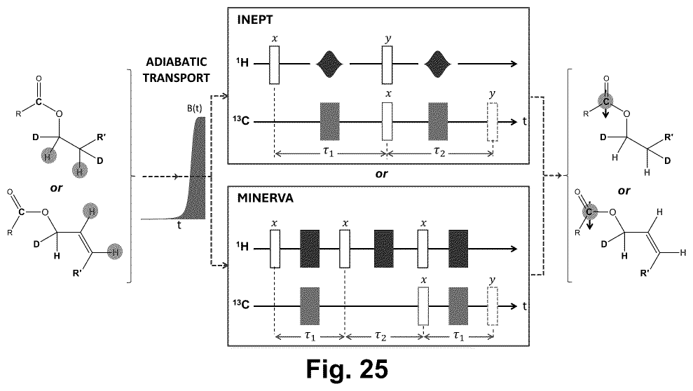

We demonstrate the LACADENA-sINEPT protocol using a modified version of the apparatus described in Ref. 6. Briefly, a dual syringe pump (Chemyx 4000) is employed to synchronize infusion of the reactant solution into the reaction chamber with the withdrawal of liquid hydrogenation products into a Varian 400 MHz flow NMR probe depicted in FIG. 15 of the supporting information. The tubing connecting the withdrawing syringe to the reactor outlet, including the fluid path incorporating the NMR flow cell, is initially filled with neat acetone-d₆, and the reaction chamber is pressurized to 6 bars with 98% para-enriched H₂. The 20 mL infusing syringe is filled with a precursor solution containing 10 mM Rh(cod)(dppb)BF₄ and 40 mM AP or APd substrate in acetone-d₆. Details of the syntheses of AP and APd are provided in Ref.⁶ The precursor solution was infused at a flow rate of 5 mL/min through a section of 1/16 inch O.D. copper tubing wound around a brass block fitted with a thermocouple and a cartridge-type heating element to heat the liquid to the desired reaction temperature before entering the ultrasonic nozzle into the reaction chamber, The hydrogenation was performed in the fringe field of the wide-bore (89 mm) Bruker Ultrashield superconducting magnet where the magnetic field was measured to be approximately 2.5 mT.

The ultrasonic nozzle, excited at 120 kHz, produces a fine spray of precursor droplets with a mean diameter of about 13 μm.²⁶ The liquid hydrogenation adducts accumulate at the bottom of the funnel-shaped reaction chamber. After 5 s of infusion, the withdrawing syringe is activated to draw the solution into the magnetic field and NMR flow cell. The withdrawing syringe flow rate of 3 mL/min is employed to balance polarization losses due to spin relaxation against non-adiabaticity during transport from the reactor to the NMR flow cell.

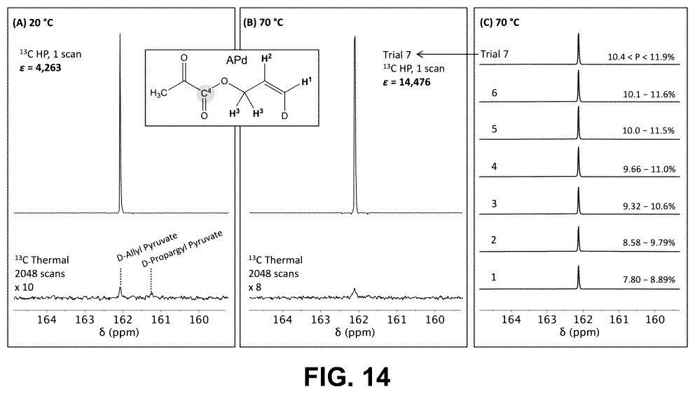

FIG. 14 presents the LACADENA-sINEPT ¹³C NMR spectra acquired at 9.4 T after hydrogenation of APd at either 20 or 70° C. The corresponding thermally polarized ¹³C spectra were acquired after waiting 10 min to allow complete relaxation of the hyperpolarization. The ¹³C signal enhancements and spin polarizations were calculated from the signal integrals using Eq. S1-S3, which includes an Ernst condition correction.

For hydrogenation at 70° C., the hyperpolarized ¹³C signals are seen to gradually increase over the course of the seven trials. This could be due to increasing activity of the catalyst over the course of the experiment. A maximum P(¹³C) hyperpolarization of between 10.4 and 11.9% is estimated. The range represents the due to the uncertainty in the 2^{nd} hydrogenation to propyl ester. The lower limit is obtained if it is assumed that the 2^{nd} hydrogenation occurred mainly inside the NMR flow cell after acquisition of the HP ¹³C spectrum. The upper limit of 11.9% assumes that hydrogenation was complete prior to acquisition of the HP ¹³C spectrum. This systematic error is similar to the statistical error of the measurement.

The observed P(¹³C) levels are only about 28% of the theoretical maximum. The losses are attributed mainly to spin-lattice relaxation during accumulation of adducts and transport to high field. To our knowledge, the low-field singlet lifetime Tₛ (i.e., the lifetime of the singlet-triplet imbalance) has not been measured for APd, but is likely to be significantly longer than the high field spin-lattice relaxation time of the protons at 9.4 T (c.a., T₁=6.4 s).

### Conclusions

We have demonstrated a new hybrid spin order transfer process combining (1) ultrasonic spray injection hydrogenation of a selectively deuterated propargyl pyruvate precursor, (2) adiabatic longitudinal transport through a level anti-crossing, and (3) selective INEPT coherence transfer across the ester linkage. Pyruvate carbonyl ¹³C polarizations of up to 11±1% were recorded, which is higher than what has been reported for two-step coherence transfer pulse sequences or magnetic field cycling schemes. Notably, the deuteration pattern in the doubly deuterated propargyl ester (e.g., APd₂) is expected to automatically increase the efficiency by a factor of two, according to symmetry considerations and numerical density matrix simulations. It could be more than a factor of two if the spin-lattice relaxation time of the single CHD proton in APd₂ is longer than that of the CH₂ proton pair in APd. An advantage of the LACADENA-sINEPT approach over relayed coherence transfer sequences for propargyl SAH is that coherence transfer is achieved in a single step, thereby reducing τ₂ decoherence losses. On the other hand, high efficiency requires a short transport time that maintains adiabaticity. Per-deuterated vinyl ester precursors can be expected to exhibit higher polarizations due to the exceptionally long ¹H spin relaxation time. For ethyl esters, substantial coupling between a pH₂-source proton and the carbonyl ¹³C affords the highest attainable polarization in the absence of LAC-induced SOT. On the other hand, the propargyl ester is a more stable and more easily synthesized precursor with higher reaction rates. The ALTADENA/LACADENA-INEPT process is versatile, being applicable to both vinyl and propargyl side-arms.

From a technical perspective, the LACADENA-INEPT experimental arrangement offers practical advantages over the standard PASADENA-based batch reaction processes utilizing in-situ bubbling of pH₂ through the NMR sample tube. First, hydrogenation is performed outside of the NMR magnet, where the design and construction of a high-performance hydrogenation reactor, such as our ultrasonic spray injection reactor, is not constrained by the limited space within the bore of the NMR magnet. Secondly, the standard INEPT pulse sequence affords a straightforward setup and optimization using ordinary Zeeman spin polarization at thermal equilibrium. Moreover, INEPT pulse sequences are provided in the standard pulse sequence library on commercial spectrometers. Non-selective INEPT can be employed, albeit with reduced efficiency, when selective excitation is not supported by the hardware or at lower magnetic fields (FIG. 13) where frequency selective excitation is infeasible.

Numerical simulations indicate that ¹³C polarizations of up to 90% are possible using LACADENA-sINEPT in the doubly deuterated APd₂ ester. At the present time, only APd was available. Nevertheless, extrapolation of our experimental results to APd₂ suggests that polarization levels well over 20% are attainable (pre-cleavage). This compares favorably with dissolution DNP,^{27,28} but with substantially lower cost, higher reproducibility, and shorter polarization times.
1 C. R. Bowers and D. P. Weitekamp, J Am Chem Soc, 1987, 109, 5541-5542.
2 C. R. Bowers and D. P. Weitekamp, Phys Rev Lett, 1986, 57, 2645-2648.
3 R. W. Adams, J. A. Aguilar, K. D. Atkinson, M. J. Cowley, P. I. P. Elliott, S. B. Duckett, G. G. R. Green, I. G. Khazal, J. Lopez-Serrano and D. C. Williamson, Science (1979), 2009, 323, 1708-1711.
4 J. B. Hovener, A. N. Pravdivtsev, B. Kidd, C. R. Bowers, S. Gloggler, K. v. Kovtunov, M. Plaumann, R. Katz-Brull, K. Buckenmaier, A. Jerschow, F. Reineri, T. Theis, R. v. Shchepin, S. Wagner, P. Bhattacharya, N. M. Zacharias and E. Y. Chekmenev, Angewandte Chemie International Edition, 2018, 57, 11140-11162.
5 A. B. Schmidt, C. R. Bowers, K. Buckenmaier, E. Y. Chekmenev, H. de Maissin, J. Eills, F. Ellermann, S. Glöggler, J. W. Gordon, S. Knecht, I. v Koptyug, J. Kuhn, A. N. Pravdivtsev, F. Reineri, T. Theis, K. Them and J.-B. Hövener, Anal Chem, 2022, 94, 479-502.
6 M.-J. Ferrer, E. L. Kuker, E. Semenova, A. J. Gangano, M. P. Lapak, A. J. Grenning, V. M. Dong and C. R. Bowers, J Am Chem Soc, 2022, 144, 20847-20853.
7 G. A. Morris and R. Freeman, J Am Chem Soc, 2002, 101, 760-762.
8 M. G. Pravica and D. P. Weitekamp, Chem Phys Lett, 1988, 145, 255-258.
9 H. J. Hogben, M. Krzystyniak, G. T. P. Charnock, P. J. Hore and I. Kuprov, Journal of Magnetic Resonance, 2011, 208, 179-194.
10 A. Bax, Journal of Magnetic Resonance (1969), 1984, 57, 314-318.
11 A. B. Schmidt, A. Brahms, F. Ellermann, S. Knecht, S. Berner, J. Hennig, D. Von Elverfeldt, R. Herges, J. B. Hövener and A. N. Pravdivtsev, Physical Chemistry Chemical Physics, 2021, 23, 26645-26652.
12 E. Cavallari, C. Carrera, S. Aime and F. Reineri, Chemistry—A European Journal, 2017, 23, 1200-1204.
13 E. Cavallari, C. Carrera, S. Aime and F. Reineri, Journal of Magnetic Resonance, 2018, 289, 12-17.
14 F. Reineri, T. Boi and S. Aime, Nat Commun, 2015, 6, 5858.
15 R. v. Shchepin, D. A. Barskiy, A. M. Coffey, I. v. Manzanera Esteve and E. Y. Chekmenev?, Angewandte Chemie International Edition, 2016, 55, 6071-6074.
16 E. Cavallari, C. Carrera, M. Sorge, G. Bonne, A. Muchir, S. Aime and F. Reineri, Sci Rep, 2018, 8, 8366.
17 N. v. Chukanov, O. G. Salnikov, R. v. Shchepin, K. v. Kovtunov, I. v. Koptyug and E. Y. Chekmenev, ACS Omega, 2018, 3, 6673-6682.
18 M. C. D. Tayler and L. F. Gladden, Journal of Magnetic Resonance, 2019, 298, 101-106.
19 A. Marshall, A. Salhov, M. Gierse, C. Müller, M. Keim, S. Lucas, A. Parker, J. Scheuer, C. Vassiliou, P. Neumann, F. Jelezko, A. Retzker, J. W. Blanchard, I. Schwartz and S. Knecht, ArXiv, DOI:10.48550/arxiv.2205.15709.
20 A. Svyatova, V. P. Kozinenko, N. v Chukanov, D. B. Burueva, E. Y. Chekmenev, Y.-W. Chen, D. W. Hwang, K. v Kovtunov and I. v Koptyug, Sci Rep, 2021, 11, 5646.
21 N. J. Stewart, H. Kumeta, M. Tomohiro, T. Hashimoto, N. Hatae and S. Matsumoto, Journal of Magnetic Resonance, 2018, 296, 85-92.
22 S. Korchak, S. Yang, S. Mamone and S. Glöggler, ChemistryOpen, 2018, 7, 344-348.
23 L. Dagys, A. P. Jagtap, S. Korchak, S. Mamone, P. Saul, M. H. Levitt and S. Glöggler, Analyst, 2021, 146, 1772-1778.
24 M. H. Levitt, Journal of Magnetic Resonance, 2016, 262, 91-99.
25 N. C. Nielsen, T. Schulte-Herbrüggen and O. W. Sørensen, Mol Phys, 1995, 85, 1205-1216.
26 Sono-Tek product information, https://www.sono-tek.com, (accessed 10 Feb. 2023).
27 J. H. Ardenkjaer-Larsen, Journal of Magnetic Resonance, 2016, 264, 3-12.
28 J. H. Ardenkjaer-Larsen, B. Fridlund, A. Gram, G. Hansson, L. Hansson, M. H. Lerche, R. Servin, M. Thaning and K. Golman, Proc Natl Acad Sci USA, 2003, 100, 10158-10163.

### Supplemental Information for Example 4

### Abbreviations

PPP=propyl 2-oxopropanoate-1-¹³C; AP=allyl 2-oxopropanoate-1-¹³C; APd=(Z)-allyl-3-d 2-oxopropanoate-1-¹³C; APd2=(Z)-allyl-1,3-d₂ 2-oxopropanoate-1-¹³C; PPGP=prop-2-yn-1-yl 2-oxopropanoate-1-¹³C

### Experimental Details

### Instrumentation

Para-enriched H₂ gas (pH₂) was generated by flowing normal H₂ gas (Airgas, UHP) through a cryocooled parahydrogen converter (Advanced Research Systems, Inc. M/N-DE204AF) with a catalyst compartment packed with 46 g of FeO(OH) (Sigma-Aldrich) cooled to 20 K. Para-enrichment to >90% was confirmed by ¹H NMR spectroscopy by comparison to normal H₂ gas stored at 300 K.

Hydrogenation of the propargyl substrates with pH₂ was performed using a home-built ultrasonic spray injection reactor system based on a 120 kHz, 3 W Sono-tek ultrasonic nozzle (P/N 06-04-00003-010). The nozzle was clamped to an aluminum reaction chamber with a KF-40 flange. The reactor assembly was positioned about 40 cm to the side of the lower bore opening of the 9.4 T wide-bore Bruker Ultrashield™ Superconducting magnet where the fringe field was measured to be 2.4 mT. Heating tape connected to a Variac variable transformer was wrapped around the aluminum reaction chamber body, and a thermocouple was inserted between the wrappings to monitor the chamber temperature. The first 20 mL (infusion) syringe of the programmable Chemyx 4000 dual syringe pump was connected by approximately 35 cm of 0.020-inch I.D. ( 1/16-inch O.D.) PEEK tubing to 0.020-inch I.D. ( 1/16-in O.D) copper tubing using a flangeless IDEX union connector. The copper tubing, approximately 46 inches in length, was coiled 13 times around a cylindrical brass heating mantle (1-inch O.D.) to provide good thermal contact for heating the flowing precursor solution. The outlet of the copper tubing was connected to the inlet of the ultrasonic nozzle, creating a fine spray in the pressurized chamber. A 75 psi back pressure regulator (IDEX, Part #: U-606) was inserted between the heated copper coil and the ultrasonic nozzle inlet to prevent the H₂ gas from expanding into the syringe before or after infusion. The outlet at the bottom of the funnel-shaped reactor was connected to the inlet of port the Varian IFC NMR probe using the same type of PEEK tubing. The total length of the tubing between the detection cell of the NMR probe (including the PEEK tubing internal to the IFC probe) and the outlet of the reactor chamber is approximately 155 cm in length. The second 20 mL (withdrawing) syringe was connected to the outlet port of the Varian IFC NMR probe by 97 cm of 0.030-inch I.D. ( 1/16-inch O.D.) PEEK tubing.

sINEPT Experimental Protocol

A stock solution of a 1:4 molar ratio of 10 mM Rh(cod)(dppb)BF₄ (Sigma-Aldrich) catalyst and 40 mM substrate in acetone-d₆ (Cambridge Isotope Labs, 99.98%) was sparged with nitrogen gas for 2 minutes. Next, the infusing syringe was loaded with the degassed precursor solution (3.75 mL catalyst stock solution: 11.25 mL substrate stock solution). Before the experiment, the PEEK tubing and IFC NMR probe were pre-filled with deuterated solvent up to the bottom of the funnel-shaped reaction chamber. The aluminum reaction chamber could then be pressurized to 6 bars with pH₂. For heated reactions, the brass mantle and chamber were heated to 70° C.

The ¹³C NMR spectra were acquired on a Varian VNMRS spectrometer fitted with a Varian 400 MHz Triple-Resonance IFC (interchangeable flow cell) NMR probe with a detection volume of 60 μL. The Varian flow probe was installed in the 9.4 T superconducting magnet. The ¹H 90° pulse duration was 2.25 μs, and the ¹³C 90° pulse duration was 9.12 μs. Chemical shifts of the hydrogenation reaction mixture are given with respect to acetone-d₆.

To set up the acquisition of the hyperpolarized sINEPT ¹³C NMR spectrum, an ALTADENA-mode spectrum was acquired using ultrasonic spray injection hydrogenation according to our previously published protocol¹. After allowing the hyperpolarized spin order to fully relax, the thermally polarized ¹H spectrum was used to set up the selective excitation of the methylene protons of the allyl product molecule using a rsnob-shaped pulse with ¹H reference pulse width of 2.25 μs and power of 60 dB. A frequency span of 50 Hz centered at the methylene proton resonance frequency was used to ensure selective excitation of the methylene protons of the allyl side arm. This corresponds to a shaped pulse width of 37 ms at a power level of −3 dB. In the Varian VNMRJ software, the tau delay in the pulse sequence is calculated from the 3.5 Hz J-coupling between the methylene protons and ¹³C of the substrate.

For acquisition of hyperpolarized ¹³C NMR spectra, a 1 mL aliquot of the 4:1 substrate:catalyst mixture with a Rh(cod)(dppb)BF₄ concentration of 10 mM in d₆-acetone was infused at a rate of 5 mL/min through the ultrasonic nozzle into the reaction chamber which was pressurized to 6 bars with >90% para-enriched H₂. After 0.5 mL of the pH₂ adduct solution had accumulated at the bottom of the reactor, the second syringe pump was activated to draw the liquid into the Varian NMR flow probe at syringe withdrawal rate of 3 mL/min, corresponding to a transport time of about 6 s. The hyperpolarized ¹³C NMR spectra were acquired upon application of the selective INEPT pulse sequence.

After acquisition of the hyperpolarized ¹³C spectrum, the spin polarization was allowed to relax to thermal equilibrium over a period of 10 minutes. The thermally polarized ¹³C spectrum was acquired by co-addition of 2048 scans (30° pulse, Ernst angle) using a relaxation delay of 5 seconds, a gain of 60, and an acquisition time of 1 second.

sINEPT Pulse Sequence Experimental Optimization

### Tau Refocusing Delay Dependence

The tau value used in the sINEPT pulse sequence was optimized on a 250 mM allyl ¹³C-acetate solution in acetone-d₆. Figure S1 presents the results of the arrayed sINEPT ¹³C experiments acquired by independent incrementation of τ₁ and τ₂. The spectra were acquired by accumulating 16 transients using a relaxation delay of 30 seconds. The optimized pulse sequence, sINEPT-tau, allows for the shaped pulse timing accommodation where

$$\frac{{τ}_{1}}{2} = 52 ms$$

$$and$$

$$\frac{{τ}_{2}}{2} = 17$$

ms (noting that τ/2 is the time between the soft π and hard π/2 pulses).
Enhancement Factor and Polarization Calculation (Spray Injection sINEPT)

The enhancement factor ε_{HP}^{13C} obtained for the sINEPT signals was calculated as the ratio of the integrals of the hyperpolarized ¹³C signal, S_{HP}^{13C}, and the thermally polarized signal, S_{Therm}^{13C}. If S_{Therm}^{13C} is accumulated under “Ernst Angle” conditions:

$${ε}_{HP}^{13 C} = \frac{{S}_{HP}^{13 C}}{{S}_{Therm}^{13 C}} \times \frac{{ns}_{Therm}}{{ns}_{HP}} \times R \qquad Equation S1$$

$$where$$

$$R = \frac{sin ( {β}_{Ernst} )}{1 + cos ( {β}_{Ernst} )}$$

Here, ns_{Therm} represents the number of scans for the thermally polarized spectrum (either 1024, 2048, or 4096), ns_{HP} is a single scan for the hyperpolarized spectrum in the sINEPT experiment. The factor R, where β_{Ernst}=arccos(e^{-τ/T}^{₁}), where τ is the recycle delay, is applied for accumulation of the thermally polarized spectrum under the Ernst conditions.

If there is further hydrogenation of (Z)-allyl-3-d 2-oxopropanoate-1-¹³C (APd) after acquisition of the HP ¹³C-APd signal, the signal enhancement of ¹³C (APd) will be overestimated and this introduces a systematic error in the enhancement factor and any other quantity derived from it. The correction for the change in APd concentration is:

$${ε}_{HP , corr}^{13 C} = {ε}_{HP}^{13 C} \times \frac{1}{( 1 + {X}_{PPP} )} \qquad Equation S2$$

Under our experimental conditions, the amount of conversion to PPP that occurs after acquisition of the HP signal is uncertain. Therefore, we estimate the enhancement factor in two limiting assumptions:

Case (a): X_{PPP}=0 in Equation S2. There is negligible second hydrogenation to PPP after the HP ¹³C spectrum has been acquired.

Case (b): X_{PPP}>0 in Equation S2. Second hydrogenation to PPP occurs inside the NMR flow cell after acquisition of the HP ¹³C spectrum.

The thermal equilibrium nuclear spin polarization, P₀^{13C} is calculated using Equation S3.

$${P}_{0}^{13 C} = tan h ( \frac{{ℏγ}_{13 C} {B}_{0}}{2 {k}_{b} T} ) \qquad Equation S3$$

A 9.4 T magnetic field at the ambient lab temperature of 293 K yields P₀=8.21×10⁻⁶.

The hyperpolarized spin polarization, P_{HP}^{13C}, is then calculated from Equation S4.

$${P}_{HP}^{13 C} = ( {ε}_{HP}^{13 C} \times {P}_{0}^{13 C} ) \qquad Equation S4$$

In practice, measurement of the extent of second hydrogenation is problematic due to: (1) the proton signals of the terminal methine group are not present in the deuterated form; (2) there is spectral overlap between the unreacted precursor peaks with the product peaks. Under the assumption that deuteration does not significantly affect the rate of hydrogenation, data from the hydrogenation of the non-deuterated prop-2-yn-1-yl 2-oxopropanoate-1-¹³C (PPGP) formed under the same reaction conditions was used to estimate the fractional conversion, X_{PPP}. The conversion to PPP is reported in Table S1 is averaged across four trials.

### Conversion Calculation

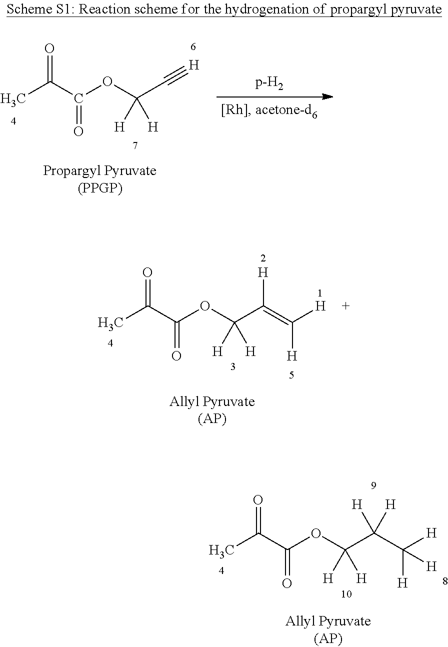

The fractional conversion from propargyl pyruvate to AP (X_{AP}) and PPP (X_{PPP}) was calculated using Equations S5 and S6, respectively.

$${X}_{AP} = \frac{{S}_{therm}^{{H}^{1 , 5}} / 2}{{S}_{therm}^{{H}^{6}} + {S}_{therm}^{{H}^{1 , 5}} / 2 + {S}_{therm}^{{H}^{8}} / 3} \qquad Equation S5$$

$${X}_{PPP} = \frac{{S}_{therm}^{{H}^{8}} / 3}{{S}_{therm}^{{H}^{6}} + {S}_{therm}^{{H}^{1 , 5}} / 2 + {S}_{therm}^{{H}^{8}} / 3} \qquad Equation S6$$

where Sₜₕₑᵣₘ^{H}^{⁶} is the integral of thermally polarized CH signal (H⁶) in propargyl pyruvate, Sₜₕₑᵣₘ^{H}^{^{1,5}} are the integrals of thermally polarized CH₂ signal (H¹ and H⁵) in allyl pyruvate, and Sₜₕₑᵣₘ^{H}^{⁸} is the integral of the thermally polarized CH₃ signal (H⁸) in PPP. A spline correction to the baseline was applied prior to signal integration.

TABLE S1
Conversion data for ultrasonic spray injection of propargyl
pyruvate at 20° C. and 70° C. Uncertainties across the
four trials represent standard deviations.

| | % Conversion at 20° C. | | % Conversion at 70° C. | |
|---|---|---|---|---|
| | | PPGP → | | PPGP → |
| | PPGP → | PPP | PPGP → | PPP |
| | AP | (Xₚₚₚ) | AP | (Xₚₚₚ) |
| Trial 1 | 59.8% | 12.3% | 81.6% | 13.7% |
| Trial 2 | 63.4% | 7.27% | 80.4% | 12.7% |
| Trial 3 | 64.0% | 6.42% | 83.2% | 11.1% |
| Trial 4 | 64.4% | 6.46% | 77.5% | 18.8% |
| Average | 62.9 ± 1.8% | 8.1 ± 2.4% | 80.7 ± 2.1% | 14.1 ± 2.9% |

### Numerical MATLAB Spinach Simulations

### Numerical Density Matrix Simulations

Hilbert space density matrix simulations were performed using the Spinach library in Mathworks Matlab, version 2021a. Source code is available upon reasonable request. The chemical shift and J-coupling arrays used in the simulation of AP, APd, and APd₂ are as follows:

### APd

% Basis set
bas.formalism=‘zeeman-hilb’;
bas.approximation=‘none’;
sys.isotopes={‘1H’,‘1H’,‘1H’,‘13C’,‘1H’,‘1H’};
inter.zeeman.scalar={5.99 5.28 4.72 0 5.4 4.72};
% Scalar couplings
inter.coupling.scalar{1,2}=10.53;
inter.coupling.scalar{1,3}=5.6;
inter.coupling.scalar{1,4}=−0.18;
inter.coupling.scalar{1,5}=17.2;
inter.coupling.scalar{1,6}=5.6;
inter.coupling.scalar{2,3}=1.25;
inter.coupling.scalar{2,4}=0.2;
inter.coupling.scalar{2,5}=1.46;
inter.coupling.scalar{2,6}=1.25;
inter.coupling.scalar{3,4}=3.15;
inter.coupling.scalar{3,5}=1.58;
inter.coupling.scalar{3,6}=10.0; % geminal coupling
inter.coupling.scalar{4,5}=0.08;
inter.coupling.scalar{4,6}=3.15;
inter.coupling.scalar{5,6}=1.58;
inter.coupling.scalar{6,6}=0.0;
parameters.offset=[1000 0];
parameters.spins={‘1H’,‘13C’};
parameters.nspins=6;

### APd

% Basis set
bas.formalism=‘zeeman-hilb’;
bas.approximation=‘none’;
% SELECTIVE INVERSION PULSES IN RELAY AND INEPT BLOCKS
% 6 spin system
sys.isotopes={‘1H’,‘1H’,‘1H’,‘13C’,‘2H’,‘1H’};
inter.zeeman.scalar={5.99 5.28 4.72 0 0 4.72};
% Scalar couplings
inter.coupling.scalar{1,2}=10.53;
inter.coupling.scalar{1,3}=5.6;
inter.coupling.scalar{1,4}=−0.18;
inter.coupling.scalar{1,5}=2.64;
inter.coupling.scalar{1,6}=5.6;
inter.coupling.scalar{2,3}=1.25;
inter.coupling.scalar{2,4}=0.2;
inter.coupling.scalar{2,5}=0.224;
inter.coupling.scalar{2,6}=1.25;
inter.coupling.scalar{3,4}=3.15;
inter.coupling.scalar{3,5}=0.242;
inter.coupling.scalar{3,6}=10.0; % geminal coupling
inter.coupling.scalar{4,5}=0.01228;
inter.coupling.scalar{4,6}=3.15;
inter.coupling.scalar{5,6}=0.2425;
inter.coupling.scalar{6,6}=0.0;
parameters.offset=[1000 0 0];
parameters.spins={‘1H’,‘13C’,‘2H’};
parameters.nspins=6;

### APd2

parameters.rhofile=‘APD2’;
% Basis set
bas.formalism=‘zeeman-hilb’;
bas.approximation=‘none’;
sys.isotopes={‘1H’,‘1H’,‘1H’,‘13C’,‘2H’,‘2H’};
inter.zeeman.scalar={5.99 5.28 4.72 0 0 0};
% Scalar couplings
inter.coupling.scalar{1,2}=10.53;
inter.coupling.scalar{1,3}=5.6;
inter.coupling.scalar{1,4}=−0.18;
inter.coupling.scalar{1,5}=2.64;
inter.coupling.scalar{1,6}=0.8596;
inter.coupling.scalar{2,3}=1.25;
inter.coupling.scalar{2,4}=0.2;
inter.coupling.scalar{2,5}=0.224;
inter.coupling.scalar{2,6}=0.1919;
inter.coupling.scalar{3,4}=3.15;
inter.coupling.scalar{3,5}=0.24253;
inter.coupling.scalar{3,6}=1.535; % geminal coupling
inter.coupling.scalar{4,5}=0.01228;
inter.coupling.scalar{4,6}=0.4835;
inter.coupling.scalar{5,6}=0.0;
inter.coupling.scalar{6,6}=0.0;
parameters.offset=[1000 0 0];
parameters.spins={‘1H’,‘13C’,‘2H’};
parameters.nspins=6;

The kinetic distribution of molecular hydrogenation events was simulated by time-averaging of the matrix representation of the parahydrogen projection operator over 2000 time-steps, each with a duration of 1 ms. This is sufficient to kill the off-diagonal elements in the matrix representation in the eigenbasis.

The evolution of the spin system during the flow from the hydrogenation reactor to the NMR flow cell was simulated by numerical propagation of the density matrix using the measured field profile of the 9.4 T wide-bore (89 mm) Bruker Biospin Ultrashield superconducting magnet. The profile was measured at 1 cm intervals along the flow path using a Lakeshore 421 Gaussmeter. A Microsoft Excel spreadsheet containing the field profile is available upon reasonable request. To simulate the quasi-continuous field ramp resulting from the flow of the liquid through 0.020-inch I.D. PEEK tubing, the field profile was interpolated into 4000 uniformly spaced time steps, and the density matrix was propagated using a time-independent Hamiltonian for each step. After the field ramp, the density operator was propagated through the (refocused) INEPT or selective INEPT pulse sequences, as presented in the main manuscript. A final 7/2 pulse was applied to the ¹³C spin to store the polarization, calculated from the density matrix p using trace relation, P(¹³C)=2 Tr{Ŝ_{z}·\hat{ρ}}, where Ŝ_{z} is the z-projection spin operator. For sINEPT transfer from proton Zeeman order on allyl CH₂ protons (in AP or APd), coherence transfer is optimized using τ₁=(2J_{CH})⁻¹ and τ₂=τ₁/2. For CDH groups (e.g., APd₂), τ₁=T2=(2J_{CH})⁻¹ is used. In sINEPT, frequency selective shaped 7 pulses are applied to the allyl methylene proton(s), H³.

### Experimental Hyperpolarized Spectra

### Allyl 2-oxopropanate-1-¹³C in acetone-d₆ (AP)

¹³C Spin-Lattice Relaxation Time Measurement on Allyl 2-Oxopropanoate-1-¹³C (AP)

A standard inversion-recovery pulse sequence was used to measure the spin-lattice relaxation times starting from thermal equilibrium nuclear spin polarization of AP that were performed on the reaction product solution several hours after completing the spray-injection hyperpolarization experiments. The relaxation delay was set to 300 seconds to ensure relaxation and an acquisition of 2 seconds. The results are presented in Figure S9.

### Summary of ¹³C Polarization Results

TABLE S2
Average ¹³C polarizations P_{HP}^{13C} and standard deviations for AP and APd
at 20° C. and 70° C. obtained with and without application of the
correction factor (1 + X_{PPP})⁻¹ (see Section 4) for second hydrogenation.

| | ¹³C Polarization at 20° C. | | ¹³C Polarization at 70° C. | |
|---|---|---|---|---|
| | AP | APd | AP | APd |
| X_{PPP} = 0 | 0.79 ± 0.1% | 3.45 ± 0.1% | 0.52 ± 0.1% | 10.7 ± 1% |
| X_{PPP} (Table S1) | 0.69 ± 0.1% | 3.19 ± 0.1% | 0.46 ± 0.1% | 9.42 ± 1% |

### REFERENCES

1. Ferrer, M.-J.; Kuker, E. L.; Semenova, E.; Gangano, A. J.; Lapak, M. P.; Grenning, A. J.; Dong, V. M.; Bowers, C. R. Adiabatic Passage through Level Anticrossings in Systems of Chemically Inequivalent Protons Incorporating Parahydrogen: Theory, Experiment, and Prospective Applications. J Am Chem Soc 2022, 144 (45), 20847-20853.

1. A method for preparing a hydrogenation adduct molecule comprising at least one hyperpolarized heteronucleus, the method comprising:
(a) providing a supply of parahydrogen;
(b) hydrogenating a precursor molecule comprising at least one carbon-carbon double bond or triple bond and at least one heteronucleus using the supply of parahydrogen to form a hydrogenated analyte comprising at least a first parahydrogen atom and a second parahydrogen atom, wherein, during hydrogenation, at least one carbon-carbon double bond is reduced to a carbon-carbon single bond, or wherein at least one carbon-carbon triple bond is reduced to a carbon-carbon double bond;
(c) transferring spin from the first parahydrogen atom or the second parahydrogen atom to a third hydrogen atom and transferring spin from the third hydrogen atom to at least one heteronucleus to produce the hyperpolarized heteronucleus; or
(d) transferring spin from the first or second hydrogen atom directly to the heteronucleus, in molecules where the third hydrogen atom is not present.

2. The method of claim 1, wherein the hydrogenating is conducted in a magnetic field of from about 0 to about 2 T.

3. The method of claim 1, wherein the hydrogenating comprises contacting the precursor molecule and the supply of parahydrogen with a hydrogenation catalyst, wherein the hydrogenation catalyst comprises Pt, Pd, Cu, Au, Ag, Rh, Ru, Ir, Ni, Sn, Co, Zn, Ce, Ti, Al, Fe, Si, or any combination thereof.

4. The method of claim 3, wherein the hydrogenation catalyst is Rh or a Rh alloy.

5. The method of claim 1, wherein the precursor molecule comprises an unsaturated ester of a C₁ to Cₙ carboxylic acid, where n is 2 to 4.

6. The method of claim 5, wherein the unsaturated ester comprises a vinyl ester or a propargyl ester.

7. The method of claim 1, wherein at least one heteronucleus of the precursor molecule comprises a carbon atom double bonded to an oxygen atom.

8. The method of claim 1, wherein the precursor molecule further comprises at least one deuterium.

9. The method of claim 1, wherein the spin is transferred from the first parahydrogen atom or the second parahydrogen atom to the third hydrogen atom using adiabatic passage.

10. The method of claim 9, wherein adiabatic passage is accomplished via exposing the precursor molecule to a detection magnetic field following the hydrogenating, wherein the detection magnetic field has a strength greater than about 2 T.

11. The method of claim 1 wherein the spin is transferred from the third hydrogen atom to at least one heteronucleus using the insensitive nuclei enhanced by polarization transfer (INEPT) pulse sequence or MINERVA pulse sequence, wherein the method is theoretically at least 80% efficient, wherein the method enhances a magnetic resonance signal for at least one heteronucleus of at least a factor of 100,000.

12. A system for producing a fluid sample comprising a hyperpolarized heteronucleus, comprising:
a precursor solution introduction device, wherein the precursor solution comprises a target substrate molecule and a catalyst;
a reaction chamber, wherein the precursor solution introduction device is configured to be in fluid communication with the reaction chamber;
a gas introduction system in communication with the reaction chamber, wherein the gas introduction system is configured to introduce parahydrogen into the reaction chamber, wherein the system is configured to contact the precursor solution with the parahydrogen, resulting in the formation of a hyperpolarized fluid sample;
an adiabatic transport tube, wherein the adiabatic transport tube is configured to receive the hyperpolarized fluid sample from the reaction chamber and wherein hyperpolarized fluid sample is subjected to an increase in magnetic field, resulting in a transfer of hyperpolarization from at least one parahydrogen atom in the target substrate to a second hydrogen atom in the target substrate; and
a sample chamber in fluid communication with the adiabatic transport tube, wherein the hyperpolarized fluid sample is subjected to a high magnetic field, resulting in transfer of hyperpolarization from the second hydrogen to a heteronucleus and generating a target molecule comprising the hyperpolarized heteronucleus.

13. The system of claim 12, wherein the reaction chamber comprises an ultrasonic nozzle, and wherein the precursor solution introduction device is configured to deliver the precursor solution to the surface of the ultrasonic nozzle, wherein the ultrasonic nozzle is configured to produce droplets of the precursor solution, wherein the droplets of the precursor solution have an average diameter of from about 1 to about 50 μm in a droplet distribution range of about 25 μm or less.

14. The system of claim 12, further comprising a detection device, wherein the sample chamber containing the fluid sample comprising a hyperpolarized heteronucleus is configured to fit into the detection device, wherein the detection device is a nuclear magnetic resonance spectrometer or a magnetic resonance imaging device.

15. The system of claim 12, wherein the fluid sample is a homogeneous fluid including the target substrate molecule and a catalyst, wherein the catalyst is Rh or a Rh alloy, and wherein the catalyst is supported, tethered, a ligand stabilized in solution, or any combination thereof.

16. The system of claim 12, wherein the target substrate molecule comprises a metabolite or derivative thereof, wherein the metabolite or derivative thereof can be administered in vitro or to a subject in vivo.

17. The system of claim 16, wherein the metabolite or derivative thereof comprises an unsaturated ester of a C₁ to C₄ carboxylic acid.

18. The system of claim 17, wherein the unsaturated ester comprises a vinyl ester or a propargyl ester.

19. The system of claim 15, wherein the metabolite or derivative thereof comprises vinyl acetate, propargyl pyruvate, or any combination thereof.

20. The system of claim 12, wherein the at least one heteronucleus of the precursor molecule is a carbon-13, nitrogen-15, phosphorous-31, or fluorine-19 atom.
